SEQUENCE LISTING (16 sequences)

SEQ ID NO: 1 - protein, 14 aa, Artificial Sequence
  BCA7-2C5 (VL - CDR1)
TGTSSAHGGHYYVS

SEQ ID NO: 2 - protein, 7 aa, Artificial Sequence
  BCA7-2C5 (VL - CDR2)
DVSNRPS

SEQ ID NO: 3 - protein, 10 aa, Artificial Sequence
  BCA7-2C5 (VL - CDR3)
GSYTSSGSYV

SEQ ID NO: 4 - protein, 5 aa, Artificial Sequence
  BCA7-2C5 (VH - CDR1)
TAWMS

SEQ ID NO: 5 - protein, 19 aa, Artificial Sequence
  BCA7-2C5 (VH - CDR2)
RIKSKSDGGTTDYAAPVKG

SEQ ID NO: 6 - protein, 9 aa, Artificial Sequence
  BCA7-2C5 (VH - CDR3)
AKGGGTYGY

SEQ ID NO: 7 - protein, 110 aa, Artificial Sequence
  BCA7-2C5 light chain variable sequence
QSVLTQPASVSGSPGQSVTISCTGTSSAHGGHYYVSWYQQHPGKAPKLMIYDVSNRPSGVSNRFSGSKSGNTASLTISGLQAEDEADYYCGSYTSSGSYVFGTGTKLTVL

SEQ ID NO: 8 - protein, 118 aa, Artificial Sequence
  BCA7-2C5 heavy chain variable sequence; BCA7-2E1 heavy chain variable sequence
EVQLVESGGGLVKPGGSLRLSCAASGFTSSTAWMSWVRQAPGKGLEWVGRIKSKSDGGTTDYAAPVKGRFTISRDDSKNTLFLQMNSLKTEDTAVYYCAKGGGTYGYWGQGTTVTVSS

SEQ ID NO: 9 - protein, 14 aa, Artificial Sequence
  BCA7-2E1 (VL - CDR1)
TGTSSDGGGHTYVS

SEQ ID NO: 10 - protein, 7 aa, Artificial Sequence
  BCA7-2E1 (VL - CDR2)
DVSNRPS

SEQ ID NO: 11 - protein, 10 aa, Artificial Sequence
  BCA7-2E1 (VL - CDR3)
GSYTSSGSYV

SEQ ID NO: 12 - protein, 5 aa, Artificial Sequence
  BCA7-2E1 (VH - CDR1)
TAWMS

SEQ ID NO: 13 - protein, 19 aa, Artificial Sequence
  BCA7-2E1 (VH - CDR2)
RIKSKSDGGTTDYAAPVKG

SEQ ID NO: 14 - protein, 9 aa, Artificial Sequence
  BCA7-2E1 (VH - CDR3)
AKGGGTYGY

SEQ ID NO: 15 - protein, 110 aa, Artificial Sequence
  BCA7-2E1 light chain variable sequence
QSALTQPASVSGSPGQSVTISCTGTSSDGGGHTYVSWYQQHPGKAPKLMIYDVSNRPSWVSNRFSGSKSGNTASLTISGLQAEDEADYYCGSYTSSGSYVFGTGTKLTVL

SEQ ID NO: 16 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

(19) United States
(12) Patent Application Publication
(10) Pub. No.: US 2024/0181073 A1
(43) Pub. Date: Jun. 6, 2024

(54) Antibody-Drug Conjugates Comprising an Anti-BCMA Antibody

(71) Applicants: Sorrento Therapeutics, Inc., San Diego, CA (US); Levena (Suzhou) Biopharma Co., Ltd., Suzhou, Jiangsu (CN); Levena Biopharma US, Inc., San Diego, CA (US)

(72) Inventors: Tong Zhu, San Diego, CA (US); Alisher B. Khasanov, San Diego, CA (US); Hui Li, San Diego, CA (US); Maojun Guo, Suzhou (CN); Yanwen Fu, San Diego, CA (US); Yufeng Hong, San Diego, CA (US)

(21) Appl. No.: 18/279,768

(22) PCT Filed: Mar. 2, 2022
(86) PCT No.: PCT/CN2022/078738

§ 371 Date: Aug. 31, 2023

(30) Foreign Application Priority Data
Mar. 3, 2021 (WO) PCT/CN2021/078886
May 24, 2021 (WO) PCT/CN2021/095379
Feb. 23, 2022 (WO) PCT/CN2022/077512

(51) Int. Cl. A61K 47/68; A61K 47/54; A61P 35/00; C07K 5/02; C07K 7/02

(52) U.S. Cl. CPC A61K 47/6849; A61K 47/545; A61K 47/6811; A61P 35/00; C07K 5/0215; C07K 7/02

(57) ABSTRACT

Provided, inter alia, are antibody drug conjugates (ADCs) which specifically bind B Cell Maturation Antigen (BCMA). Further disclosed are pharmaceutical compositions, and methods for treating cancer.

### CROSS-REFERENCE TO RELATED APPLICATIONS

This application claims priority to International Application No. PCT/CN2021/078886, filed on Mar. 3, 2021, International Application No. PCT/CN2021/095379, filed on May 24, 2021, and International Application No. PCT/CN2022/077512, filed on Feb. 23, 2022, the disclosures of which are hereby incorporated by reference in their entireties.

Throughout this application various publications, patents, and/or patent applications are referenced. The disclosures of the publications, patents and/or patent applications are hereby incorporated by reference in their entireties into this application in order to more fully describe the state of the art to which this disclosure pertains.

### SEQUENCE LISTING

The present application is filed with a Sequence Listing in electronic format. The Sequence Listing is provided as a file entitled “2022-02-23_01223-0089-00PCT_Seq_List_ST25.txt” created on Feb. 23, 2022, which is 7,908 bytes in size. The information in the electronic format of the sequence listing is incorporated herein by reference in its entirety.

### TECHNICAL FIELD

The present disclosure relates to antibody drug conjugates (ADCs) comprising an anti-BCMA antibody and methods of making and using the same.

### INTRODUCTION AND SUMMARY

Antibody-Drug Conjugates (ADCs) allow for the targeted delivery of a drug moiety to a tumor, and, in some embodiments intracellular accumulation therein, where systemic administration of unconjugated drugs may result in unacceptable levels of toxicity to normal cells (Polakis P. (2005) Current Opinion in Pharmacology 5:382-387). ADCs are targeted chemotherapeutic molecules which combine properties of both antibodies and cytotoxic drugs by targeting potent cytotoxic drugs to antigen-expressing tumor cells (Teicher, B. A. (2009) Current Cancer Drug Targets 9:982-1004), thereby enhancing the therapeutic index by maximizing efficacy and minimizing off-target toxicity (Carter, P. J. and Senter P. D. (2008) The Cancer Jour. 14(3):154-169: Chari, R. V. (2008) Acc. Chem. Res. 41:98-107.

The present disclosure provides ADCs comprising an anti-BCMA antibody conjugated to the drug moiety through linker moieties. In embodiments, the anti-BCMA antibody binds to BCMA-expressing cancer cells and allows for selective uptake of the ADC into the cancer cells. In embodiments, the ADCs provided herein selectively deliver an effective amount of drug moiety to tumor tissue and reduce the non-specific toxicity associated with related ADCs. The ADC compounds described herein include those with anticancer activity.

B Cell Maturation Antigen (BCMA), also known as TNFRSF17 and CD269 (UniProt Q02223), is a member of the tumor necrosis receptor superfamily. BCMA is a non-glycosylated type III transmembrane protein that is expressed on differentiated plasma cells (Laabi et al., 1992 The EMBO Journal 11(11):3897-3904; Laabi et al., 1994 Nucleic Acids Research 22(7):1147-1154; Madry et al., 1998 International Immunology 10(11):1693-1702) and is a cell surface receptor that is involved in B cell development and survival.

BCMA is a cell surface receptor for two ligands of the TNF superfamily, APRIL (A PRoliferation-Inducing Ligand) and BAFF. APRIL and BAFF are high and low affinity ligands to BCMA, respectively. APRIL is a proliferation-inducing ligand and BAFF is a B lymphocyte stimulator. TACI is a negative regulator that binds APRIL and BAFF. The coordinated binding of APRIL and BAFF to BCMA and/or TACI induces transcription of factor NF-uB and increases expression of pro-survival Bcl-2 family members and down regulates expression of pro-apoptotic factors which promotes survival and inhibits apoptosis. This complex interaction promotes B cell differentiation, proliferation, survival, and antibody production (Rickert 2011 Immunology Review 244(1):115-133). BCMA is known to support growth and survival of malignant human B cells, and upregulated expression of BCMA and TACI has been reported in malignant human B cells including multiple myeloma (MM) cells (see review in “BAFF and APRIL: a tutorial on B cell survival” by Mackay et al., 2004 Annual Review Immunology 21:231-264). Additionally, BCMA, APRIL and BAFF signaling have been reported to activate NFκB in B cell neoplasms and multiple myeloma.

Multiple myeloma is a clonal B-cell lymphoma that develops in multiple sites in the bone marrow then spreads through circulation. BCMA expression (both transcript and protein) is reported to correlate with disease progression in multiple myeloma. Thus, BCMA is expressed at significantly higher levels in multiple myeloma cells compared to normal tissues, making BCMA a good target antigen for immunotherapy. Thus, antibody drug conjugates (ADCs) where the drug is conjugated to anti-BCMA antibodies, can provide a very targeted and potent anti-tumor activity.

In one aspect, provided herein are antibody-drug conjugates (ADCs) comprising a monoclonal antibody. In another aspect, provided herein are antibody-drug conjugates (ADCs) comprising an anti-BCMA, anti-ROR1, anti-CD25, or anti-Claudine 18 antibody. In another aspect, provided herein are methods of preparing ADCs comprising a monoclonal antibody. In another aspect, provided herein are methods of preparing ADCs comprising an anti-BCMA, anti-ROR1, anti-CD25, or anti-Claudine 18 antibody. In another aspect, provided herein are precursor compounds. Also provided herein are methods for treating cancers, such as BCMA-expressing cancers, using the ADCs disclosed herein.

In embodiments, the present disclosure provides an antibody drug conjugate (ADC), having an IgG antibody that binds to a BCMA target, conjugated at the cysteine sites of the IgG antibody. In embodiments, the present disclosure provides an antibody drug conjugate (ADC), having an IgG antibody that binds to a BCMA target, conjugated at the lysine sites of the IgG antibody. The present disclosure further provides a method for treating multiple myeloma comprising providing an effective amount of a BCMA ADC.

In one aspect, provided herein is an antibody drug conjugate (ADC) of formula (I):

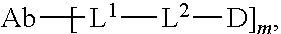

or a pharmaceutically acceptable salt thereof, wherein Ab is an anti-BCMA, anti-ROR1, anti-CD25, or anti-Claudine 18 antibody; m is an integer from 1 to 8; L¹ is a linker bound to the anti-BCMA antibody; L² is a bond, —C(O)—, —NH—, Amino Acid Unit, —(CH₂CH₂O)ₙ, —(CH₂)ₙ, -(4-aminobenzyloxycarbonyl)-,

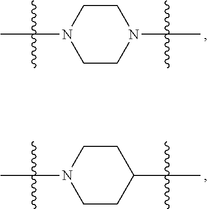 [SMILES: *N1CCN(*)CC1 ; *C1CCN(*)CC1]

—(C(O)CH₂CH₂NH)—, or combinations thereof; wherein n is an integer from 1 to 24; and D is a drug moiety.

In an aspect, provided herein is a method of treating a BCMA-expressing cancer in a subject in need thereof, said method including administering the ADC described herein (including in an aspect, embodiment, table, example, or claim), or a pharmaceutically acceptable salt thereof, to the subject.

In an aspect, provided herein is a method of preparing an antibody drug conjugate (ADC) of formula (I):

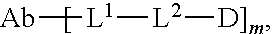

or a pharmaceutically acceptable salt thereof, said method including reacting an anti-BCMA, anti-ROR1, anti-CD25, anti-Claudine 18 antibody, or a modified antibody with a molecule of formula (P-I): B-L²-D or a pharmaceutically acceptable salt thereof, wherein B is a reactive moiety capable of forming a bond with the anti-BCMA, anti-ROR1, anti-CD25, anti-Claudine 18 antibody or a modified antibody; L² is a bond, —C(O)—, —NH—, Amino Acid Unit, —(CH₂CH₂O)ₙ—, —(CH₂)ₙ—,

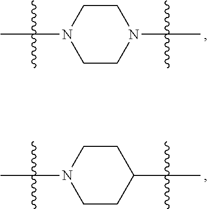 [SMILES: *N1CCN(*)CC1 ; *C1CCN(*)CC1]

-(4-aminobenzyloxycarbonyl)-, —(C(O)CH₂CH₂NH)— or combinations thereof, where n is an integer from 1 to 24; and D is a drug moiety.

In another aspect, provided herein is a compound of formula (II):

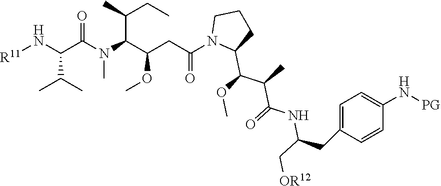

or a pharmaceutically acceptable salt thereof, wherein PG is an amine protecting group; R¹¹ is H or one or more Amino Acid Units; R¹² is H or a substituted alkyl, substituted heteroalkyl, substituted heterocycloalkyl, —CO(CH₂CH₂O)ₛCH₂CH₂U, or —CONH(CH₂CH₂O)ₛCH₂CH₂U; and wherein s is an integer from 1 to 24; and U is —NH₂, —OH, —COOH, or —OCH₃.

In any embodiment disclosed herein, the monoclonal antibody can be an anti-BCMA antibody.

### BRIEF DESCRIPTION OF THE DRAWINGS

FIG. 1A-C show results of an in vitro efficacy study of anti-BCMA-AB1-1 (shown with solid squares), anti-BCMA-AB1-2 (shown with solid circles), and anti-BCMA-AB1-3 (shown with solid triangles) using: A) NCI-H929 (BCMA+) cells; B) MM.1R (BCMA+) cells; and C) K562 (BCMA −) cells.

FIG. 2A shows results of an in vitro efficacy study of anti-BCMA-AB1-3 (shown with solid circles), anti-BCMA-AB1-4 (shown with solid triangles), anti-BCMA-AB1-5 (shown with upside down solid triangles), anti-BCMA-AB1-6 (shown with solid diamonds), anti-BCMA-AB1-7 (shown with open squares), anti-BCMA-AB1-8 (shown with open circles), and a control anti-BCMA-AB1 (shown with solid squares) using: NCI-H929 (BCMA+) cells; MM.1R (BCMA+) cells; and K562 (BCMA −) cells.

FIG. 2B shows results of an in vitro efficacy study of anti-BCMA-AB2-3 (shown with solid circles), anti-BCMA-AB2-4 (shown with solid triangles), anti-BCMA-AB2-5 (shown with upside down solid triangles), anti-BCMA-AB2-6 (shown with solid diamonds), anti-BCMA-AB2-7 (shown with open squares), anti-BCMA-AB2-8 (shown with open circles), and a control anti-BCMA-AB2 (shown with solid squares) using: NCI-H929 (BCMA+) cells; MM.1R (BCMA+) cells; and K562 (BCMA −) cells.

FIG. 3 shows results of an in vivo efficacy study in NCI-H929 xenograft in SCID beige mice of anti-BCMA-AB1-3 (2 mg/kg: shown with upside down open triangles, 4 mg/kg: shown with open triangles; 8 mg/kg: shown with solid squares) and a control anti-BCMA-AB1 (2 mg/kg: shown with open squares; 4 mg/kg: shown with X; 8 mg/kg: shown with solid triangles). PBS/vehicle (shown with open circles). *P<0.0001, two-way ANOVA with Tukey's test on tumor volumes at end points to PBS/vehicle or anti-BCMA-AB1.

FIG. 4 shows results of an in vivo efficacy study in OPM2 xenograft in SCID beige mice of anti-BCMA-AB1-3 (2 mg/kg: shown with solid squares; 0.67 mg/kg: shown with open upside-down triangles) and anti-BCMA-AB1 (2 mg/kg: shown with open squares). PBS/vehicle (shown with open circles). *P<0.0001, two-way ANOVA with Tukey's test on tumor volumes at end points to PBS/vehicle or anti-BCMA-AB1.

FIG. 5 shows results of an in vivo efficacy study in NCI-H929 xenograft in SCID beige mice of anti-BCMA-AB1-3 (1 mg/kg: shown with open triangles; 2 mg/kg: shown with open upside-down triangles; 4 mg/kg: shown with open diamonds, 8 mg/kg: shown with solid triangles) and iso-3 (1 mg/kg: shown with open squares; 2 mg/kg: shown with right half black solid and left half open squares; 4 mg/kg: shown with left half black solid and right half open squares; 8 mg/kg: shown with solid squares). PBS/vehicle (shown with open circles). *P<0.0001, two-way ANOVA with Tukey's test on tumor volumes at end points to PBS/vehicle or iso-3.

FIG. 6 shows results of an in vivo efficacy study in NCI-H929 xenograft in SCID beige mice of anti-BCMA-AB2-3 (2 mg/kg: shown with open upside-down triangles; 4 mg/kg: shown with solid triangles) and anti-BCMA-AB2 (4 mg/kg: shown with solid circles). PBS/vehicle (shown with open circles). *P<0.0001, two-way ANOVA with Tukey's test on tumor volumes at end points to PBS/vehicle or anti-BCMA-AB2.

FIGS. 7A-M show results of in vivo toxicity study in rats. FIG. 7A shows body weight change in toxin treated rats. FIG. 7B-M show hematological changes in toxin treated rats on day 7 and day 14. FIG. 7B shows white blood cell count in toxin treated rats on day 7 and 14. FIG. 7C shows neutrophil count in toxin treated rats on day 7 and 14. FIG. 7D shows percent change in neutrophils in toxin treated rats on day 7 and 14. FIG. 7E shows lymphocyte count in toxin treated rats on day 7 and 14. FIG. 7F shows eosinophil count in toxin treated rats on day 7 and 14. FIG. 7G shows monocyte count in toxin treated rats on day 7 and 14. FIG. 7H. shows reticulocyte count in toxin treated rats on day 7 and 14. FIG. 7I. shows percent change in reticulocytes in toxin treated rats on day 7 and 14. FIG. 7J. shows red blood cell count in toxin treated rats on day 7 and 14. FIG. 7K. shows hemoglobin concentration in toxin treated rats on day 7 and 14. FIG. 7L. shows percent change in hematocrit in toxin treated rats on day 7 and 14. FIG. 7M. shows platelet count in toxin treated rats on day 7 and 14.

FIG. 8 shows results of an in vitro efficacy study of ADC-50, ADC-51, ADC-52, ADC-53, ADC-3 (in all cases anti-BCMA AB1 clone was used), and controls anti-BCMA antibody (AB1 clone), anti-RSV antibody, anti-RSV antibody conjugated with Compound 3, and D3 toxin using: NCI-H929 (BCMA+) cells and K562 (BCMA −) cells.

### DETAILED DESCRIPTION OF THE INVENTION

### Definitions

Unless defined otherwise, technical and scientific terms used herein have meanings that are commonly understood by those of ordinary skill in the art unless defined otherwise. Generally, terminologies pertaining to techniques of cell and tissue culture, molecular biology, immunology, microbiology, genetics, transgenic cell production, protein chemistry and nucleic acid chemistry and hybridization described herein are well known and commonly used in the art. The methods and techniques provided herein are generally performed according to conventional procedures well known in the art and as described in various general and more specific references that are cited and discussed herein unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992). A number of basic texts describe standard antibody production processes, including, Borrebaeck (ed) Antibody Engineering, 2nd Edition Freeman and Company, N Y, 1995: McCafferty et al. Antibody Engineering, A Practical Approach IRL at Oxford Press, Oxford, England, 1996; and Paul (1995) Antibody Engineering Protocols Humana Press, Towata, N.J., 1995; Paul (ed.), FundamentalImmunology, Raven Press, N.Y, 1993; Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Coding Monoclonal Antibodies: Principles and Practice (2nd ed.) Academic Press, New York, N.Y., 1986, and Kohler and Milstein Nature 256: 495-497, 1975. All of the references cited herein are incorporated herein by reference in their entireties. Enzymatic reactions and enrichment/purification techniques are also well known and are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are well known and commonly used in the art. Standard techniques can be used for chemical syntheses, chemical analyses, pharmaceutical preparation, formulation, and delivery, and treatment of patients.

The headings provided herein are not limitations of the various aspects of the disclosure, which aspects can be understood by reference to the specification as a whole.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative (e.g., “or”) herein is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition. In embodiments, about includes the specified value.

In this disclosure, “comprises,” “comprising,” “containing” and “having” and the like can have the meaning ascribed to them in U.S. Patent law and can mean “includes,” “including,” and the like. “Consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The terms “polypeptide,” “peptide” and “protein” and other related terms used herein are used interchangeably to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety. Polypeptides include mature molecules that have undergone cleavage. These terms encompass native and artificial proteins, protein fragments and polypeptide analogs (such as muteins, variants, chimeric proteins and fusion proteins) of a protein sequence as well as post-translationally, or otherwise covalently or non-covalently, modified proteins. Two or more polypeptides (e.g., 3 polypeptide chains) can associate with each other, via covalent and/or non-covalent association, to form a multimeric polypeptide complex (e.g., multi-specific antigen binding protein complex). Association of the polypeptide chains can also include peptide folding. Thus, a polypeptide complex can be dimeric, trimeric, tetrameric, or higher order complexes depending on the number of polypeptide chains that form the complex.

As used herein, the terms “cancer,” “neoplasm,” and “tumor” are used interchangeably and, in either the singular or plural form, refer to cells that have undergone a malignant transformation that makes them pathological to the host organism. Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient. Tumors may be a hematopoietic (or hematologic or hematological or blood-related) cancer, for example, cancers derived from blood cells or immune cells, which may be referred to as “liquid tumors.” Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia, lymphomas such as non-Hodgkin's lymphoma, Hodgkin's lymphoma; and the like.

The cancer may be any cancer in which an abnormal number of blast cells or unwanted cell proliferation is present or that is diagnosed as a hematological cancer, including both lymphoid and myeloid malignancies Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia (AML). Myeloid malignancies also include myeloproliferative disorders (MIPD) which include, but are not limited to, chronic myelogenous (or myeloid) leukemia (CML), chronic myelomonocytic leukemia (CMML), essential thrombocythemia (or thrombocytosis), and polycythemia vera (PCV). Myeloid malignancies also include myelodysplasia (or mvelodvsplastic syndrome or MDS), which may be referred to as refractory anemia (RA), refractory anemia with excess blasts (RAEB), and refractory anemia with excess blasts in transformation (RAEBT); as well as myelofibrosis (MIFS) with or without agnogenic myeloid metaplasia.

Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), inmermediate-grade (or aggressive) or high-grade (very aggressive). Indolent Bcell lymphomas include follicular lymphoma (FL), small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large cell lymphoma (DL.BCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma. HIV associated (or AIDS related) lymphomas, and post-transplanm lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hlodgkin's lymphoma s (T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenstrom's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

An “antibody” and “antibodies” and related terms used herein refers to an intact immunoglobulin or to an antigen binding portion thereof that binds specifically to an antigen. Antigen binding portions may be produced by recombinant DNA techniques or by enzymatic or chemical cleavage of intact antibodies. Antigen binding portions include, inter alia, Fab, Fab′, F(ab′)₂, Fv, domain antibodies (dAbs), and complementarity determining region (CDR) fragments, single-chain antibodies (scFv), chimeric antibodies, diabodies, triabodies, tetrabodies, and polypeptides that contain at least a portion of an immunoglobulin that is sufficient to confer specific antigen binding to the polypeptide.

Antibodies include recombinantly produced antibodies and antigen binding portions. Antibodies include non-human, chimeric, humanized and fully human antibodies. Antibodies include monospecific, multispecific (e.g., bispecific, trispecific and higher order specificities). Antibodies include tetrameric antibodies, light chain monomers, heavy chain monomers, light chain dimers, heavy chain dimers. Antibodies include F(ab′)₂ fragments, Fab′ fragments and Fab fragments. Antibodies include single domain antibodies, monovalent antibodies, single chain antibodies, single chain variable fragment (scFv), camelized antibodies, affibodies, disulfide-linked Fvs (sdFv), anti-idiotypic antibodies (anti-Id), minibodies. Antibodies include monoclonal and polyclonal populations. Anti-BCMA antibodies are described herein.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

An “epitope” and related terms as used herein refers to a portion of an antigen that is bound by an antigen binding protein (e.g., by an antibody or an antigen binding portion thereof). An epitope can comprise portions of two or more antigens that are bound by an antigen binding protein. An epitope can comprise non-contiguous portions of an antigen or of two or more antigens (e.g., amino acid residues that are not contiguous in an antigen's primary sequence but that, in the context of the antigen's tertiary and quaternary structure, are near enough to each other to be bound by an antigen binding protein). Generally, the variable regions, particularly the CDRs, of an antibody interact with the epitope. Anti-BCMA antibodies, and antigen binding proteins thereof, that bind an epitope of a BCMA polypeptide are described herein.

An “antibody fragment”, “antibody portion”, “antigen-binding fragment of an antibody”, or “antigen-binding portion of an antibody” and other related terms used herein refer to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; Fd; and Fv fragments, as well as dAb; diabodies; linear antibodies; single-chain antibody molecules (e.g. scFv); polypeptides that contain at least a portion of an antibody that is sufficient to confer specific antigen binding to the polypeptide. Antigen binding portions of an antibody may be produced by recombinant DNA techniques or by enzymatic or chemical cleavage of intact antibodies. Antigen binding portions include, inter alia, Fab, Fab′, F(ab′)2, Fv, domain antibodies (dAbs), and complementarity determining region (CDR) fragments, chimeric antibodies, diabodies, triabodies, tetrabodies, and polypeptides that contain at least a portion of an immunoglobulin that is sufficient to confer antigen binding properties to the antibody fragment. Antigen-binding fragments of anti-BCMA antibodies are described herein.

An antigen binding protein can have, for example, the structure of an immunoglobulin. In one embodiment, an “immunoglobulin” refers to a tetrameric molecule. Each tetrameric molecule is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The carboxy-terminal portion of each chain defines a constant region primarily responsible for effector function. Human light chains are classified as kappa or lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two antigen binding sites. In one embodiment, an antigen binding protein can be a synthetic molecule having a structure that differs from a tetrameric immunoglobulin molecule but still binds a target antigen or binds two or more target antigens. For example, a synthetic antigen binding protein can comprise antibody fragments, 1-6 or more polypeptide chains, asymmetrical assemblies of polypeptides, or other synthetic molecules. The terms “variable heavy chain,” “VH,” or “VH” refer to the variable region of an immunoglobulin heavy chain, including an Fv, scFv, dsFv or Fab; while the terms “variable light chain,” “VL” or “VL” refer to the variable region of an immunoglobulin light chain, including of an Fv, scFv, dsFv or Fab. “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991). Antigen binding proteins having immunoglobulin-like properties that bind specifically to BCMA are described herein.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., FUNDAMENTAL IMMUNOLOGY (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny el al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

The terms “antigen binding protein” “antigen binding domain,” “antigen binding region,” or “antigen binding site” and related terms used herein refers to a protein comprising a portion that binds to an antigen and, optionally, a scaffold or framework portion that allows the antigen binding portion to adopt a conformation that promotes binding of the antigen binding protein to the antigen. Examples of antigen binding proteins include antibodies, antibody fragments (e.g., an antigen binding portion of an antibody), antibody derivatives, and antibody analogs. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129; Roque et al., 2004, Biotechnol. Prog. 20:639-654. In addition, peptide antibody mimetics (“PAMs”) can be used, as well as scaffolds based on antibody mimetics utilizing fibronection components as a scaffold. Antigen binding proteins that bind BCMA are described herein.

In one embodiment, a dissociation constant (K_{D}) can be measured using a BIACORE surface plasmon resonance (SPR) assay. Surface plasmon resonance refers to an optical phenomenon that allows for the analysis of real-time interactions by detection of alterations in protein concentrations within a biosensor matrix, for example using the BIACORE system (Biacore Life Sciences division of GE Healthcare, Piscataway, NJ).

“Specifically binds” as used throughout the present specification in relation to anti-BCMA antigen binding proteins means that the antigen binding protein binds human BCMA (hBCMA) with no or insignificant binding to other human proteins. The term however does not exclude the fact that antigen binding proteins of the invention may also be cross-reactive with other forms of BCMA, for example primate BCMA. For example, in one embodiment the antigen binding protein does not bind to TACI or BAFF-R. In one embodiment, an antibody specifically binds to a target antigen if it binds to the antigen with a dissociation constant K_{D} of 10⁻⁵ M or less, or 10⁻⁶ M or less, or 10⁻⁷ M or less, or 10⁻⁸ M or less, or 10⁻⁹ M or less, or 10⁻¹⁰ M or less.

The term “BCMA,” as used herein, refers to any native BCMA from any vertebrate source, including mammals such as primates (e.g. humans, cynomolgus monkey (cyno)) and rodents (e.g., mice and rats), unless otherwise indicated. The term encompasses “full-length,” unprocessed BCMA as well as any form of BCMA that results from processing in the cell. The term also encompasses naturally occurring variants of BCMA, e.g., splice variants, allelic variants, and isoforms. The amino acid sequence of an exemplary human BCMA protein is shown in SEQ ID NO: 16.

The term “BCMA-expressing cancer” refers to a cancer comprising cells that express BCMA on their surface.

The terms “anti-BCMA antibody” and “an antibody that binds to BCMA” refer to an antibody that is capable of binding BCMA with sufficient affinity such that the antibody is useful as a therapeutic agent in targeting BCMA. In one embodiment, the extent of binding of an anti-BCMA antibody to an unrelated, non-BCMA protein is less than about 10% of the binding of the antibody to BCMA as measured, e.g., by a radioimmunoassay (RIA). In certain embodiments, an antibody that binds to BCMA has a dissociation constant (Kd) of ≤1 μM, ≤100 nM, ≤10 nM, ≤5 nM, ≤4 nM, ≤3 nM, ≤2 nM, ≤1 nM, ≤0.1 nM, ≤0.01 nM, or ≤0.001 nM (e.g., 10⁴ M or less, e.g. from 10⁻⁸ M to 10⁻¹³ M, e.g., from 10⁻⁹ M to 10⁻¹³ M). In certain embodiments, an anti-BCMA antibody binds to an epitope of BCMA that is conserved among BCMA from different species.

The term “chimeric antibody” and related terms used herein refers to an antibody that contains one or more regions from a first antibody and one or more regions from one or more other antibodies. In one embodiment, one or more of the CDRs are derived from a human antibody. In another embodiment, all of the CDRs are derived from a human antibody. In another embodiment, the CDRs from more than one human antibody are mixed and matched in a chimeric antibody. For instance, a chimeric antibody may comprise a CDR1 from the light chain of a first human antibody, a CDR2 and a CDR3 from the light chain of a second human antibody, and the CDRs from the heavy chain from a third antibody. In another example, the CDRs originate from different species such as human and mouse, or human and rabbit, or human and goat. One skilled in the art will appreciate that other combinations are possible.

Further, the framework regions may be derived from one of the same antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody (-ies) from another species or belonging to another antibody class or subclass. Also included are fragments of such antibodies that exhibit the desired biological activity (i.e., the ability to specifically bind a target antigen). Chimeric antibodies can be prepared from portions of any of the anti-BCMA antibodies described herein.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

The term “Fc” or “Fc region” as used herein refers to the portion of an antibody heavy chain constant region beginning in or after the hinge region and ending at the C-terminus of the heavy chain. The Fc region comprises at least a portion of the CH and CH₃ regions, and may or may not include a portion of the hinge region. Two polypeptide chains each carrying a half Fc region can dimerize to form an Fc region. An Fc region can bind Fc cell surface receptors and some proteins of the immune complement system. An Fc region exhibits effector function, including any one or any combination of two or more activities including complement-dependent cytotoxicity (CDC), antibody-dependent cell-mediated cytotoxicity (ADCC), antibody-dependent phagocytosis (ADP), opsonization and/or cell binding. An Fc region can bind an Fc receptor, including FcγRI (e.g., CD64), FcγRII (e.g, CD32) and/or FcγRIII (e.g., CD16a).

“Humanized antibody” refers to an antibody having a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

The term “human antibody” refers to antibodies that have one or more variable and constant regions derived from human immunoglobulin sequences. In one embodiment, all of the variable and constant domains are derived from human immunoglobulin sequences (e.g., a fully human antibody). These antibodies may be prepared in a variety of ways, examples of which are described below, including through recombinant methodologies or through immunization with an antigen of interest of a mouse that is genetically modified to express antibodies derived from human heavy and/or light chain-encoding genes. Fully human anti-BCMA antibodies and antigen binding proteins thereof are described herein. This definition of a human antibody specifically excludes a humanized antibody comprising non-human antigen-binding residues.

The term “isolated”, means altered “by the hand of man” from its natural state, has been changed or removed from its original environment, or both. When the term “isolated” is applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis, high-performance liquid chromatography or mass spectrophotometry. A protein that is the predominant species present in a preparation is substantially purified. For example, a polynucleotide or a polypeptide naturally present in a living organism is not “isolated,” but the same polynucleotide or polypeptide separated from the coexisting materials of its natural state is “isolated”, including but not limited to when such polynucleotide or polypeptide is introduced back into a cell, even if the cell is of the same species or type as that from which the polynucleotide or polypeptide was separated.

“CDRs” are defined as the complementarity determining region amino acid sequences of an antibody which are the hypervariable domains of immunoglobulin heavy and light chains. There are three heavy chain and three light chain CDRs (or CDR regions) in the variable portion of an immunoglobulin. Thus, “CDRs” as used herein may refer to all three heavy chain CDRs, or all three light chain CDRs (or both all heavy and all light chain CDRs, if appropriate).

CDRs provide the majority of contact residues for the binding of the antibody to the antigen or epitope. CDRs of interest in this invention are derived from donor antibody variable heavy and light chain sequences, and include analogs of the naturally occurring CDRs, which analogs also share or retain the same antigen binding specificity and/or neutralizing ability as the donor antibody from which they were derived.

The CDR sequences of antibodies can be determined by the Kabat numbering system (Kabat et al, (Sequences of proteins of Immunological Interest NIH, 1987); alternatively they can be determined using the Chothia numbering system (Al-Lazikani et al., (1997) JMB 273, 927-948), the contact definition method (MacCallum R. M., and Martin A. C R. and Thornton J. M, (1996), Journal of Molecular Biology, 262 (5), 732-745) or any other established method for numbering the residues in an antibody and determining CDRs known to the skilled man in the art

Other numbering conventions for CDR sequences available to a skilled person include “AbM” (University of Bath) and “contact” (University College London) methods. The minimum overlapping region using at least two of the Kabat, Chothia, AbM and contact methods can be determined to provide the “minimum binding unit”. The minimum binding unit may be a sub-portion of a CDR.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

As used herein, the term “variant” polypeptides and “variants” of polypeptides refers to a polypeptide comprising an amino acid sequence with one or more amino acid residues inserted into, deleted from and/or substituted into the amino acid sequence relative to a reference polypeptide sequence. Polypeptide variants include fusion proteins. In the same manner, a variant polynucleotide comprises a nucleotide sequence with one or more nucleotides inserted into, deleted from and/or substituted into the nucleotide sequence relative to another polynucleotide sequence. Polynucleotide variants include fusion polynucleotides.

As used herein the term “domain” refers to a folded protein structure which has tertiary structure independent of the rest of the protein. Generally, domains are responsible for discrete functional properties of proteins and in many cases may be added, removed or transferred to other proteins without loss of function of the remainder of the protein and/or of the domain. An “antibody single variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain.

The term “cytotoxic agent” as used herein refers to a substance that inhibits or prevents a cellular function and/or causes cell death or destruction. Cytotoxic agents include, but are not limited to, radioactive isotopes (e.g., ²¹¹At, ¹³¹I, ¹²⁵I, ⁹⁰Y, ¹⁸⁶Re, ¹⁸⁸Re, ¹⁵³Sm, ²¹²Bi, ³²P, ²¹²Pb and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

A “chemotherapeutic agent” is a chemical compound useful in the treatment of a cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e. g., calicheamicin, especially calicheamicin gamma1I and calicheamicin omegaI1 (see, e.g., Agnew, Chem Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK@ polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE@, FILDESIN®); dacarbazine; mannomustine; mitobronitol; mitolactol; pipobroman; gacytosine; arabinoside (“Ara-C”); thiotepa; taxoids, e.g., paclitaxel (TAXOL®; Bristol-Myers Squibb Oncology, Princeton, N.J.), ABRAXANE™ Cremophor-free, albumin-engineered nanoparticle formulation of paclitaxel (American Pharmaceutical Partners, Schaumberg, Illinois), and docetaxel (TAXOTERE®; Rhône-Poulenc Rorer, Antony, France); chloranbucil; gemcitabine (GEMZAR®); 6-thioguanine; mercaptopurine; methotrexate; platinum analogs such as cisplatin and carboplatin; vinblastine (VELBAN@); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylornithine (DMFO); retinoids such as retinoic acid; capecitabine (XELODA@); pharmaceutically acceptable salts, acids or derivatives of any of the above; as well as combinations of two or more of the above such as CHOP, an abbreviation for a combined therapy of cyclophosphamide, doxorubicin, vincristine, and prednisolone; CVP, an abbreviation for a combined therapy of cyclophosphamide, vincristine, and prednisolone; and FOLFOX, an abbreviation for a treatment regimen with oxaliplatin (ELOXATIN™) combined with 5-FU and leucovorin.

An “antibody-drug conjugate” or “ADC” is an antibody conjugated to one or more heterologous molecule(s), including but not limited to a cytotoxic agent.

As used herein, the term “conjugated” when referring to two moieties means the two moieties are bonded, wherein the bond or bonds connecting the two moieties may be covalent or non-covalent. In embodiments, the two moieties are covalently bonded to each other (e.g. directly or through a covalently bonded intermediary). In embodiments, the two moieties are non-covalently bonded (e.g. through ionic bond(s), van der waal's bond(s)/interactions, hydrogen bond(s), polar bond(s), or combinations or mixtures thereof).

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human. In certain embodiments, the subject is an adult, an adolescent, a child, or an infant. In some embodiments, the terms “individual” or “patient” are used and are intended to be interchangeable with “subject”.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity. Alignment for purposes of determining percent amino acid sequence identity can be achieved in various ways that are within the skill in the art, for instance, with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (e.g., EMBOSS Needle or EMBOSS Water, available at www.ebi.ac.uk/Tools/psa/). Those skilled in the art can determine appropriate parameters for aligning sequences, including any algorithms needed to achieve maximal alignment over the full length of the sequences being compared. “Percentage of sequence identity” or “percent (%) [sequence] identity”, as used herein, is determined by comparing two optimally locally aligned sequences over a comparison window defined by the length of the local alignment between the two sequences. (This may also be considered percentage of homology or “percent (%) homology”.) The amino acid sequence in the comparison window may comprise additions or deletions (e.g., gaps or overhangs) as compared to the reference sequence for optimal alignment of the two sequences. Local alignment between two sequences only includes segments of each sequence that are deemed to be sufficiently similar according to a criterion that depends on the algorithm used to perform the alignment (e.g., EMBOSS Water). “identical” or percent “identity,” refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region). The percentage identity is calculated by determining the number of positions at which the identical nucleic acid base or amino acid residue occurs in both sequences to yield the number of matched positions, dividing the number of matched positions by the total number of positions in the window of comparison and multiplying the result by 100. Optimal alignment of sequences for comparison may be conducted by the local homology algorithm of Smith and Waterman (Add. APL. Math. 2:482, 1981), by the global homology alignment algorithm of Needleman and Wunsch (J. Mol. Biol. 48:443, 1970), by the search for similarity method of Pearson and Lipman (Proc. Natl. Acad. Sci. USA 85: 2444, 1988), or by inspection. GAP and BESTFIT, as additional examples, can be employed to determine the optimal alignment of two sequences that have been identified for comparison. Typically, the default values of 5.00 for gap weight and 0.30 for gap weight length are used.

A comparison of the sequences and determination of the percent identity between two polypeptide sequences, or between two polynucleotide sequences, may be accomplished using a mathematical algorithm. For example, the “percent identity” or “percent homology” of two polypeptide or two polynucleotide sequences may be determined by comparing the sequences using the GAP computer program (a part of the GCG Wisconsin Package, version 10.3 (Acceirys, San Diego, Calif.)) using its default parameters. Expressions such as “comprises a sequence with at least X % identity to Y” with respect to a test sequence mean that, when aligned to sequence Y as described above, the test sequence comprises residues identical to at least X % of the residues of Y.

In one embodiment, the amino acid sequence of a test antibody may be similar but not identical to any of the amino acid sequences of the polypeptides that make up the multi-specific antigen binding protein complexes described herein. The similarities between the test antibody and the polypeptides can be at least 95%, or at or at least 96% identical, or at least 97% identical, or at least 98% identical, or at least 99% identical, to any of the polypeptides that make up the multi-specific antigen binding protein complexes described herein. In one embodiment, similar polypeptides can contain amino acid substitutions within a heavy and/or light chain. In one embodiment, the amino acid substitutions comprise one or more conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference in its entirety. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine.

Antibodies can be obtained from sources such as serum or plasma that contain immunoglobulins having varied antigenic specificity. If such antibodies are subjected to affinity purification, they can be enriched for a particular antigenic specificity. Such enriched preparations of antibodies usually are made of less than about 10% antibody having specific binding activity for the particular antigen. Subjecting these preparations to several rounds of affinity purification can increase the proportion of antibody having specific binding activity for the antigen. Antibodies prepared in this manner are often referred to as “monospecific.” Monospecific antibody preparations can be made up of about 10%, 20%, 30%, 40%, 50⁰%, 60%, 70%, 75%, 80%, 85%, 90%, 95%, 97%, 99%, or 99.9% antibody having specific binding activity for the particular antigen. Antibodies can be produced using recombinant nucleic acid technology as described below.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “pharmaceutical formulation” refers to a preparation which is in such form as to permit the biological activity of an active ingredient contained therein to be effective, and which contains no additional components which are unacceptably toxic to a subject to which the formulation would be administered.

The term “administering”, “administered” and grammatical variants refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

An “effective amount” of an agent, e.g., a pharmaceutical formulation, refers to an amount effective, at dosages and for periods of time necessary, to achieve the desired therapeutic or prophylactic result.

The abbreviations used herein have their conventional meaning within the chemical and biological arts. The chemical structures and formulae set forth herein are constructed according to the standard rules of chemical valency known in the chemical arts.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Where substituent groups are specified by their conventional chemical formulae, written from left to right, they equally encompass the chemically identical substituents that would result from writing the structure from right to left, e.g., —CH₂O— is equivalent to —OCH₂—.

The term saccharide means carbohydrate (or sugar). In embodiments, the saccharide is a monosaccharide. In embodiments, the saccharide is a polysaccharide. The most basic unit of saccharide is a monomer of carbohydrate. The general formula is CₙH₂ₙOₙ. The term saccharide derivative means sugar molecules that have been modified with substituents other than hydroxyl groups. Examples include glycosylamines, sugar phosphates, and sugar esters. Other saccharide derivatives include for example beta-D-glucuronyl, D-galactosyl, and D-glucosyl.

The term “Charged Group” means a chemical group bearing a positive or a negative charge, such as for example phosphate, phosphonate, sulfate, sulfonate, nitrate, carboxylate, carbonate, etc. In some embodiments, a Charged Group is at least 50% ionized in aqueous solution at least one pH in the range of 5-9. In some embodiments, a Charged Group is an anionic Charged Group.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, or S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂O—CH, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in addition to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in addition to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R‘- and —R’C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-lH-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” (a wavy line) denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In embodiments, the alkylarylene group has the formula:

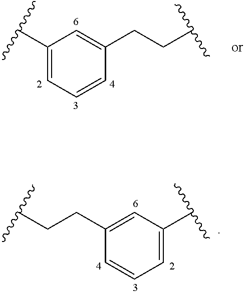 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR‘R’R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —Si R′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —N₀₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)ₚ-U-, wherein T and U are independently —NR—, —O—, —CRR′—, or a single bond, and p is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
(i) oxo, halogen, —CCl₃, —CBr₃, —CF₃, —CI₃, —CH₂CI, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHC₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
(a) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCI₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —N₀₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or“lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Linker” refers to a chemical moiety comprising a covalent bond or a chain of atoms that covalently attaches an antibody to a drug moiety. In various embodiments, linkers include a divalent radical. In various embodiments, linkers can comprise one or more amino acid residues.

“Amino Acid Unit” has the formula

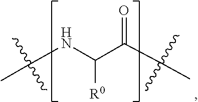

where R⁰ is hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₄NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, phenyl, or cyclohexyl. In various embodiments, Amino Acid Unit includes not only naturally occurring amino acids but also minor amino acids, and non-naturally occurring amino acid analogs, such as citrulline, norleucine, selenomethionine, β-alanine, etc. An amino acid unit may be referred to by its standard three-letter code for the amino acid (e.g., Ala, Cys, Asp, Glu, Val, Phe, Lys, etc.).

As used herein, the terms “bioconjugate” and “bioconjugate linker” refers to the resulting association between atoms or molecules of “bioconjugate reactive groups” or “bioconjugate reactive moieties”. The association can be direct or indirect. For example, a conjugate between a first bioconjugate reactive group (e.g., —NH₂, —C(O)OH, —N-hydroxysuccinimide, or -maleimide) and a second bioconjugate reactive group (e.g., thiol, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a thiol). In embodiments, the first bioconjugate reactive group (e.g., haloacetyl moiety) is covalently attached to the second bioconjugate reactive group (e.g. a thiol). In embodiments, the first bioconjugate reactive group (e.g., pyridyl moiety) is covalently attached to the second bioconjugate reactive group (e.g. a thiol). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., fluorophenyl ester moiety) reacts with the second bioconjugate reactive group (e.g. an amine) to form a covalent bond. In embodiments, the first bioconjugate reactive group (e.g., -sulfo-N-hydroxysuccinimide moiety) reacts with the second bioconjugate reactive group (e.g. an amine) to form a covalent bond.

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or thiol groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding; and
(m) metal bonding to reactive phosphorus groups (e.g. phosphines) to form, for example, phosphate diester bonds.
(n) azides coupled to alkynes using copper catalyzed cycloaddition click chemistry.
(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a thiol group.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

As used herein, common organic and cell types abbreviations are defined as follows:
Ac Acetyl
ACN Acetonitrile
Ala Alanine
Asn Asparagine
aq. Aqueous
β-Ala beta-alanine
BOC or Boc tert-Butoxycarbonyl
° C. Temperature in degrees Centigrade
CBZ Benzoxycarbonyl
Cit Citrulline
DBU 1,8-Diazabicyclo[5.4.0]undec-7-ene
DCM dichloromethane
DIEA Diisopropylethylamine
DMF N,N-Dimethylformamide
EDC 1-Ethyl-3-(3-dimethylaminopropyl)carbodiimide
EOS Eosinophils
Et Ethyl
EtOAc Ethyl acetate
Eq Equivalents
Fmoc 9-Fluorenylmethoxycarbonyl
g Gram(s)
Gly Glycine
h Hour (hours)
HATU 2-(1H-7-azabenzotriazol-1-yl)-1,1,3,3-tetramethyl uronium Hexafluorophosphate
HCT Hematocrit
HGB Hemoglobin
HOBt N-Hydroxybenzotriazole
HPLC High-performance liquid chromatography
LC/MS Liquid chromatography-mass spectrometry
LYM Lymphocytes
Lys Lysine
Me Methyl
mg milligrams
MeOH Methanol
mL Milliliter(s)
μL/μL Microliter(s)
MONO Monocytes
mol moles
mmol millimoles
μmol/μmol micromoles
MS mass spectrometry
NHS N-Hydroxysuccinimide
NEUT Neutrophils
PABC p-aminobenzyloxycarbonyl
Phe Phenylalanine
Pip piperidine
PLT Platelets
PyAOP (7-Azabenzotriazol-1-yloxy)tripyrrolidinophosphonium hexafluorophosphate
RBC Red blood cells
RET Reticulocytes
RP-HPLC reverse phase HPLC
rt room temperature
Ser Serine
t-Bu tert-Butyl
Tert, t tertiary
TFA Trifluoracetic acid
Thr Threonine
Val Valine
WBC White blood cells

Compositions

Antibody-Drug Conjugates

In one aspect, provided herein is an antibody-drug conjugate (ADC) comprising a monoclonal antibody (Ab), a drug moiety (D), and a linker moiety that covalently attaches the monoclonal antibody to the drug moiety.

In another aspect, provided herein is an ADC of formula (I):

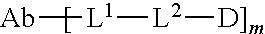

or a pharmaceutically acceptable salt thereof, wherein:
Ab is a monoclonal antibody;
m is an integer from 1 to 8;
L¹ is a linker bound to the monoclonal antibody;
L² is a bond, —C(O)—, —NH—, Amino Acid Unit, —(CH₂CH₂O)ₙ—, —(CH₂)ₙ—, -(4-aminobenzyloxycarbonyl)-,

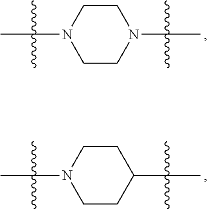 [SMILES: *N1CCN(*)CC1 ; *C1CCN(*)CC1]

—(C(O)CH₂CH₂NH)— or combinations thereof, where n is an integer from 1 to 24;
D is a drug moiety.

In another aspect, provided herein is an antibody drug conjugate (ADC) of formula (I):

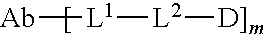

or a pharmaceutically acceptable salt thereof, wherein:
Ab is an anti-BCMA, anti-ROR1, anti-CD25, or anti-Claudine 18 antibody;
m is an integer from 1 to 8;
L¹ is a linker bound to the anti-BCMA, anti-ROR1, anti-CD25, or anti-Claudine 18 antibody;
L² is a bond, —C(O)—, —NH—, Amino Acid Unit, —(CH₂CH₂O)ₙ—, —(CH₂)ₙ—, -(4-aminobenzyloxycarbonyl)-,

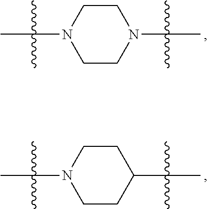 [SMILES: *N1CCN(*)CC1 ; *C1CCN(*)CC1]

—(C(O)CH₂CH₂NH)—, or combinations thereof; wherein n is an integer from 1 to 24; and
D is a drug moiety.

In embodiments, m is an integer from 1 to 8. In embodiments, m is 1. In embodiments, m is 2. In embodiments, m is 3. In embodiments, m is 4. In embodiments, m is 5. In embodiments, m is 6. In embodiments, m is 7. In embodiments, m is 8.

In embodiments, n is an integer from 1 to 24. In embodiments, n is 1. In embodiments, n is 2. In embodiments, n is 3. In embodiments, n is 4. In embodiments, n is 5. In embodiments, n is 6. In embodiments, n is 7. In embodiments, n is 8. In embodiments, n is 9. In embodiments, n is 10. In embodiments, n is 11. In embodiments, n is 12. In embodiments, n is 13. In embodiments, n is 14. In embodiments, n is 15. In embodiments, n is 16. In embodiments, n is 17. In embodiments, n is 18. In embodiments, n is 19. In embodiments, n is 20. In embodiments, n is 21. In embodiments, n is 22. In embodiments, n is 23. In embodiments, n is 24.

In embodiments, Ab is an anti-BCMA antibody, anti-ROR1 antibody, anti-CD25 antibody, or anti-Claudin 18 antibody. In embodiments, Ab is an anti-BCMA antibody. In embodiments, Ab is an anti-ROR1 antibody. In embodiments, Ab is an anti-CD25 antibody. In embodiments, Ab is an anti-Claudin 18 antibody.

In embodiments, L¹ is a linker bound to the anti-BCMA antibody. In embodiments, L¹ is a linker bound to one or two sulfur or nitrogen atoms on the anti-BCMA antibody. In embodiments, L¹ is a linker bound to one sulfur atom on the anti-BCMA antibody. In embodiments, L¹ is a linker bound to two sulfur atoms on the anti-BCMA antibody. In embodiments, L¹ is a linker bound to one nitrogen atom on the anti-BCMA antibody. In embodiments, L¹ is a linker bound to two nitrogen atoms on the anti-BCMA antibody.

In embodiments, L¹ is a linker bound to one cysteine molecule on the anti-BCMA antibody. In embodiments, L¹ is a linker bound to two cysteine molecules on the anti-BCMA antibody. In embodiments, L¹ is a linker bound to one lysine molecule on the anti-BCMA antibody. In embodiments, L¹ is a linker bound to two lysine molecules on the anti-BCMA antibody.

In embodiments, L¹ is

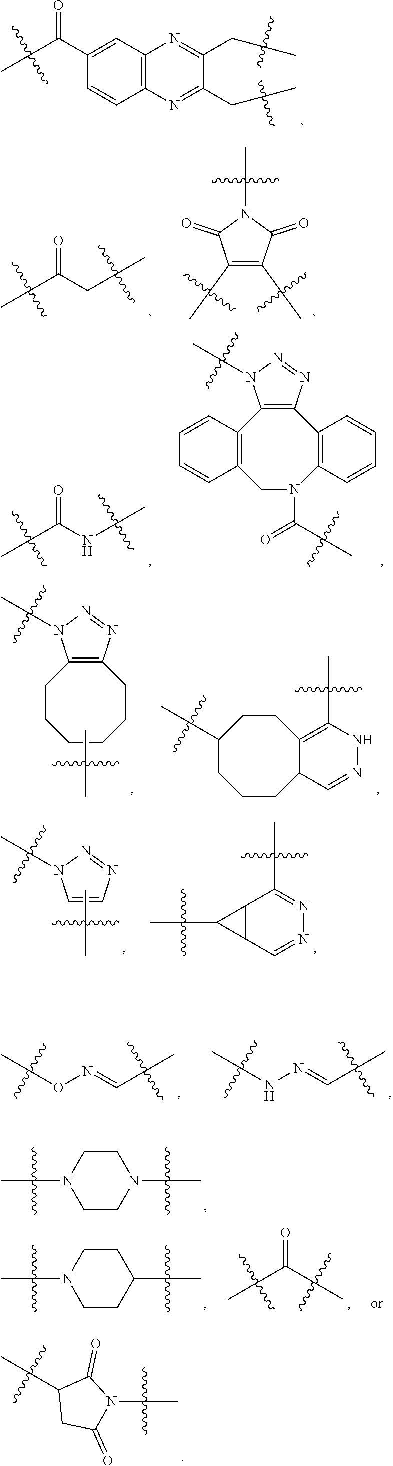

In embodiments, L¹ is

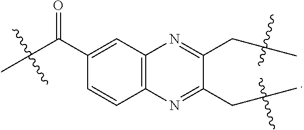 [SMILES: *Cc1nc2ccc(C(*)=O)cc2nc1C*]

In embodiments, L¹ is

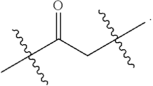 [SMILES: *CC(*)=O]

In embodiments, L¹ is

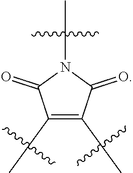 [SMILES: *C1=C(*)C(=O)N(*)C1=O]

In embodiments, L¹ is

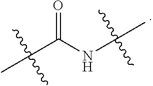 [SMILES: *NC(*)=O]

In embodiments, L¹ is

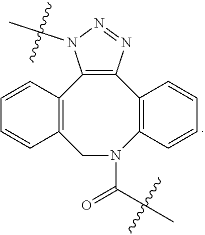 [SMILES: *C(=O)N1Cc2ccccc2-c2c(nnn2*)-c2ccccc21]

In embodiments, L¹ is

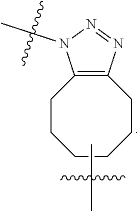

In embodiments, L¹ i

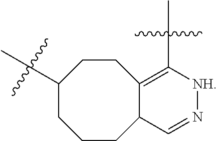 [SMILES: *C1=C2CCC(*)CCCC2C=NN1]

In embodiments, L¹ is

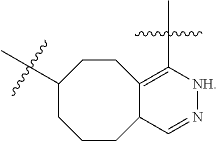 [SMILES: *C1=C2CCC(*)CCCC2C=NN1]

In embodiments, L¹ is

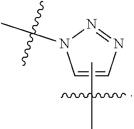

In embodiments, L¹ is

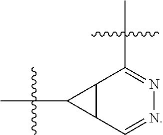 [SMILES: *C1=NN=CC2C(*)C12]

In embodiments, L¹ is

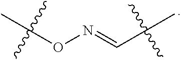 [SMILES: */C=N/O*]

In embodiments, L¹ is

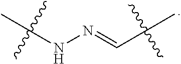 [SMILES: */C=N/N*]

In embodiments, L¹ is

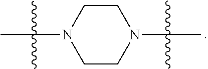 [SMILES: *N1CCN(*)CC1]

In embodiments, L¹ is

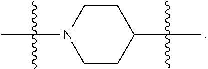 [SMILES: *C1CCN(*)CC1]

In embodiments, L¹ is

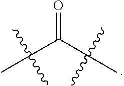 [SMILES: *C(*)=O]

In embodiments, L¹ is

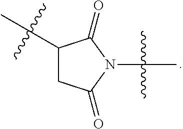 [SMILES: *C1CC(=O)N(*)C1=O]

Where L¹ is

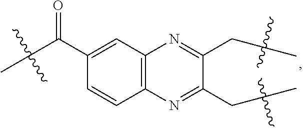 [SMILES: *Cc1nc2ccc(C(*)=O)cc2nc1C*]

the two CH₂ moieties shown on the right side of the structure may each be bound to a different cysteine of the anti-BCMA antibody via a thiol group. Where L¹ is

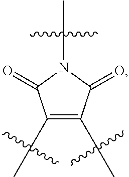 [SMILES: *C1=C(*)C(=O)N(*)C1=O]

the two alkene carbons shown on the bottom of the structure may each be bound to a different cysteine of the anti-BCMA antibody via a thiol group. Where L¹ is

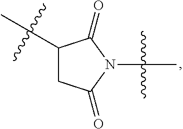 [SMILES: *C1CC(=O)N(*)C1=O]

the carbon may be bound to a cysteine of the anti-BCMA antibody via a thiol group.

In embodiments, D is:

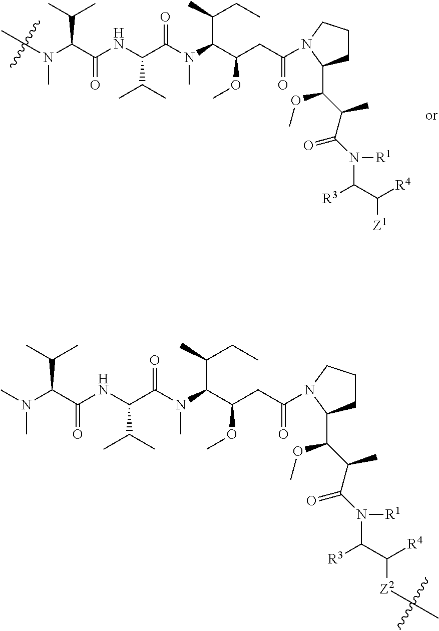

R¹ is H or —C₁-C₈ alkyl;
R³ is H, halogen, —CCl₃, —CBr₃, —CF₃, —C₁₃, —CHCI₂, —CHBr₂, —CHF₂, —CHI₂, —CH₂Cl, —CH₂Br, —CH₂F, —CH₂I, —CN, —OR^{3A}, —NR^{3A}R^{3B}, —(CH₂)ᵥOR⁶,

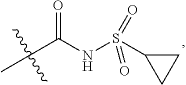 [SMILES: *C(=O)NS(=O)(=O)C1CC1]

substituted or unsubstituted alkyl, or substituted or unsubstituted heteroalkyl;
R⁴ is H, halogen, —OR^{4A}, —NR^{4A}R^{4B}, substituted or unsubstituted alkyl, or substituted or unsubstituted heteroalkyl:
Z¹ is a substituted or unsubstituted aryl, substituted or unsubstituted heteroaryl, substituted or unsubstituted cycloalkyl, or substituted or unsubstituted heterocycloalkyl;
Z² is a substituted or unsubstituted arylene, substituted or unsubstituted heteroarylene, substituted or unsubstituted cycloalkylene, or substituted or unsubstituted heterocycloalkylene;
R⁶ is H, substituted or unsubstituted alkyl, substituted or unsubstituted cycloalkyl, substituted or unsubstituted heterocycloalkyl, substituted or unsubstituted aryl, substituted or unsubstituted heteroaryl, —CO(CH₂CH₂O)_{w}CH₂CH₂Y, —CONH(CH₂CH₂O)_{w}CH₂CH₂Y,

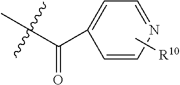

a Charged Group, or a saccharide derivative, wherein
v is an integer from 1 to 24; w is an integer from 1 to 24; Y is —NH₂, —OH, —COOH, or —OCH₃; R¹⁰ is —OH, —OCH₃ or —COOH;
each R^{3A}, R^{3B}, R^{4A}, and R^{4B} is independently H or substituted or unsubstituted alkyl.

In embodiments, L² is a bond, —C(O)—, —NH—, -Val-, -Phe-, -Lys-, -(4-aminobenzyloxycarbonyl)-,

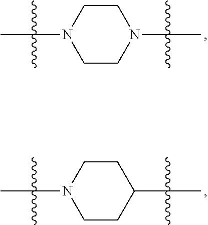 [SMILES: *N1CCN(*)CC1 ; *C1CCN(*)CC1]

-Gly-, -Ser-, -Thr-, -Ala-, -β-Ala-, -citrulline- (Cit), —(CH₂)ₙ—, —(CH₂CH₂O)ₙ—, or combinations thereof.

In embodiments, L² is a bond, —C(O)—, —NH—, -Val-, -Phe-, -Lys-, -(4-aminobenzyloxycarbonyl)-,

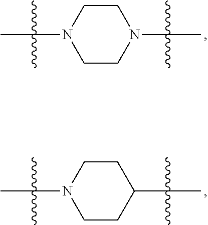 [SMILES: *N1CCN(*)CC1 ; *C1CCN(*)CC1]

—(CH₂)ₙ—, —(CH₂CH₂O)ₙ—, or combinations thereof.

In embodiments, L² is a bond, —C(O)—, —NH—, -Gly-, -Ser-, -Thr-, -Ala-, -β-Ala-, -Cit-,

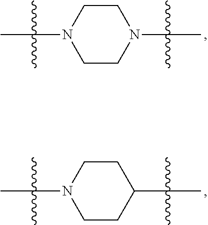 [SMILES: *N1CCN(*)CC1 ; *C1CCN(*)CC1]

—(CH₂)ₙ—, —(CH₂CH₂O)ₙ—, or combinations thereof.

In embodiments, L² is

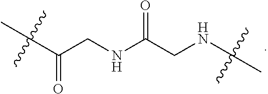 [SMILES: *NCC(=O)NCC(*)=O]

In embodiments, L¹ is NH₂

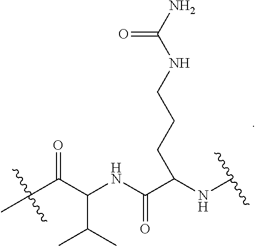 [SMILES: *NC(CCCNC(N)=O)C(=O)NC(C(*)=O)C(C)C]

In embodiments, L¹ is

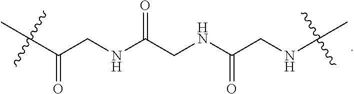 [SMILES: *NCC(=O)NCC(=O)NCC(*)=O]

In embodiments, L¹ is

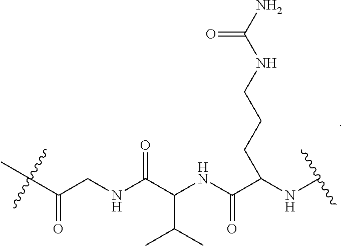 [SMILES: *NC(CCCNC(N)=O)C(=O)NC(C(=O)NCC(*)=O)C(C)C]

In embodiments, L¹ is

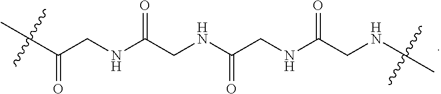 [SMILES: *NCC(=O)NCC(=O)NCC(=O)NCC(*)=O]

In embodiments, L¹ is

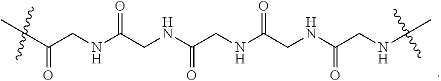 [SMILES: *NCC(=O)NCC(=O)NCC(=O)NCC(=O)NCC(*)=O]

In embodiments, L² is —C(O)—(CH₂)₅—. In embodiments, L² is

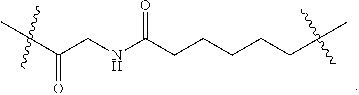 [SMILES: *CCCCCC(=O)NCC(*)=O]

In embodiments, L² is

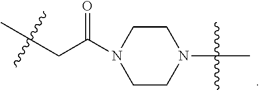 [SMILES: *CC(=O)N1CCN(*)CC1]

In embodiments, L² is

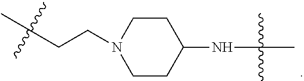 [SMILES: *CCN1CCC(N*)CC1]

In embodiments, L² is

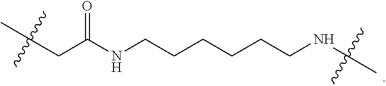 [SMILES: *CC(=O)NCCCCCCN*]

In embodiments, L² is

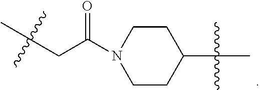 [SMILES: *CC(=O)N1CCC(*)CC1]

In embodiments, L² is a bond. In embodiments, L² is —C(O)—. In embodiments, L² is —NH—. In embodiments, L² is -Val-. In embodiments, L² is -Phe-. In embodiments, L² is -Lys-. In embodiments, L² is -(4-aminobenzyloxycarbonyl)-. In embodiments, L² is —(CH₂)ₙ—. In embodiments, L² is —(CH₂CH₂O)ₙ—. In embodiments, L² is -Gly-. In embodiments, L² is -Ser-. In embodiments, L² is -Thr-. In embodiments, L² is -Ala-. In embodiments, L² is -β-Ala-. In embodiments, L is -Cit-.

In embodiments, -L¹-L²- is

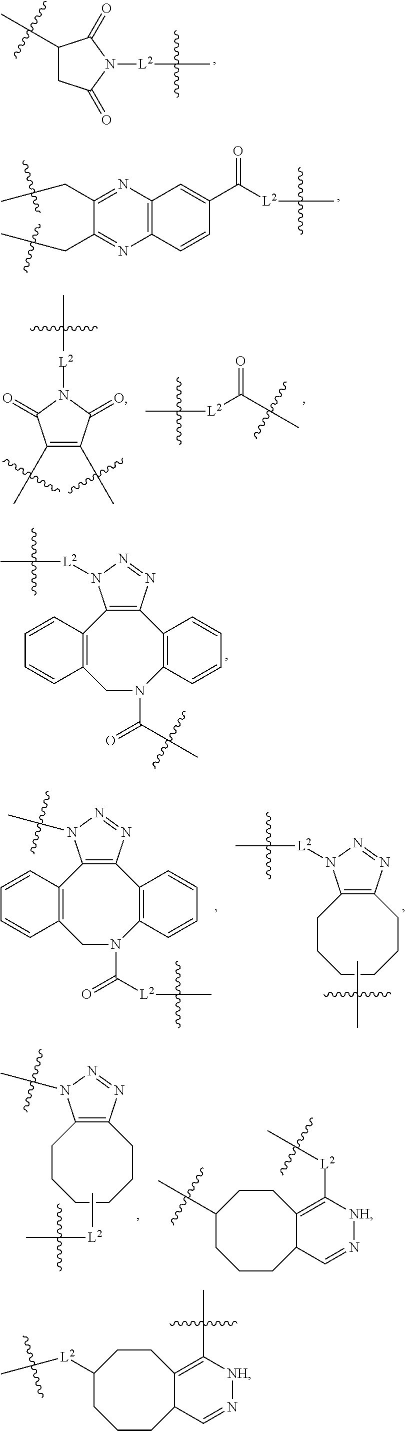

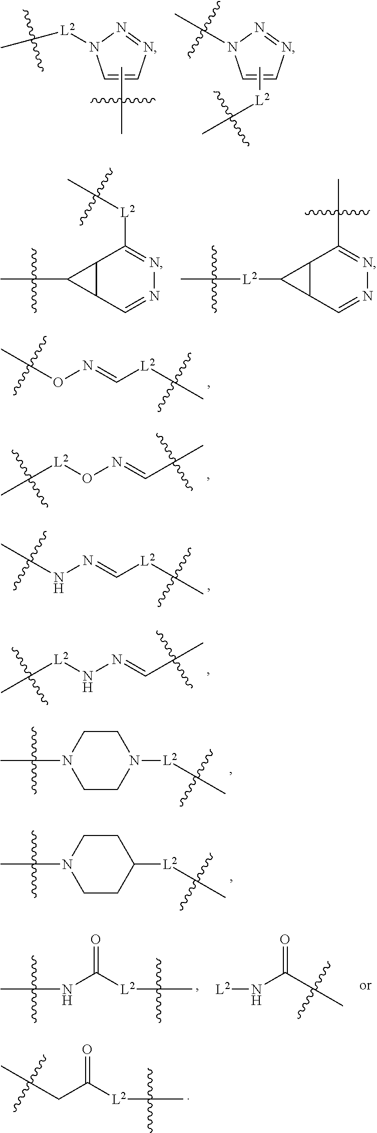

In embodiments, -L¹-L²- is

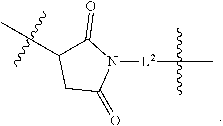

In embodiments, -L¹-L²- is

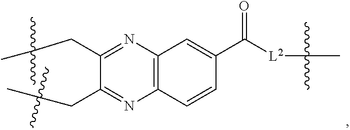

where the two CH₂ moieties shown on the left side of the structure may each be bound to a separate sulfur of the anti-BCMA antibody. In embodiments, -L¹-L²- is

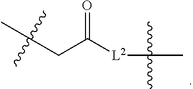

In embodiments, -L¹-L²- is

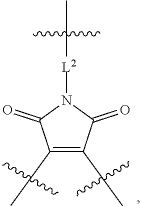

where the two alkene carbons shown on the bottom of the structure may each be bound to a separate sulfur of the anti-BCMA antibody. In embodiments, -L¹-L²- is

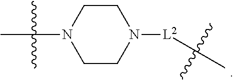

In embodiments, -L¹-L²- is

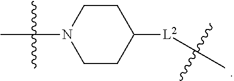

In embodiments, -L¹-L²- is

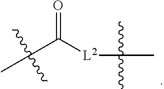

In embodiments, -L¹-L²- is

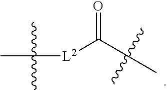

In embodiments, -L¹-L²- is

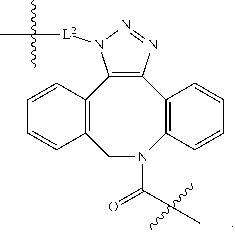

In embodiments, -L¹-L²- is

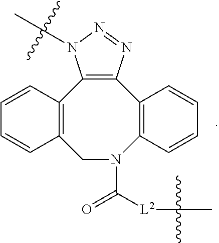

In embodiments, -L¹-L²- is

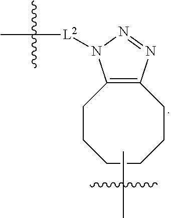

In embodiments, -L¹-L²- is

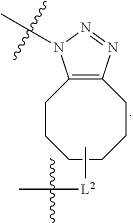

In embodiments, -L¹-L²- is

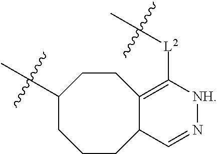

In embodiments, -L¹-L²- is

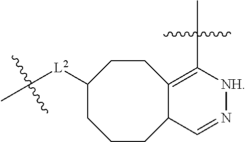

In embodiments, -L¹-L²- is

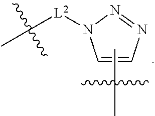

In embodiments, -L¹-L²- is

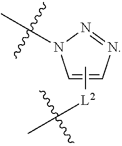

In embodiments, -L¹-L²- is

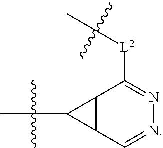

In embodiments, -L¹-L²- is

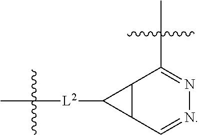

In embodiments, -L¹-L²- is

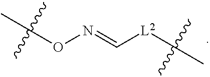

In embodiments, -L¹-L²- is

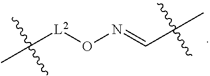

In embodiments, -L¹-L²- is

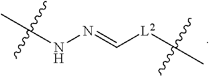

In embodiments, -L¹-L²- is

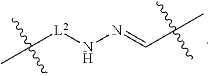

In embodiments, -L¹-L²- is

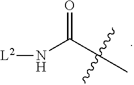

In embodiments, -L¹-L²- is

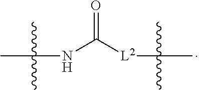

In embodiments, R¹ is H. In embodiments, R¹ is —C₁-C₈ alkyl.

In embodiments, R¹ is methyl, ethyl, propyl, isopropyl, butyl, isobutyl, tert-butyl, pentyl, or hexyl. In embodiments, R¹ is methyl. In embodiments, R¹ is ethyl. In embodiments, R¹ is propyl. In embodiments, R¹ is isopropyl. In embodiments, R¹ is butyl. In embodiments, R¹ is isobutyl. In embodiments, R¹ is tert-butyl. In embodiments, R¹ is pentyl. In embodiments, R¹ is hexyl.

In embodiments, R³ is H, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OR^{3A}, —NR^{3A}R^{3B}, —(CH₂)ᵥOR⁶,

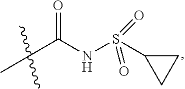 [SMILES: *C(=O)NS(=O)(=O)C1CC1]

substituted or unsubstituted alkyl (e.g., C₁-C₈ alkyl, C₁-C₆ alkyl, or C₁-C₄ alkyl), or substituted or unsubstituted heteroalkyl (e.g., 2 to 8 membered heteroalkyl, 2 to 6 membered heteroalkyl, or 2 to 4 membered heteroalkyl).

In embodiments, R³ is H, —OR^{3A}, —(CH₂)ᵥOR⁶,

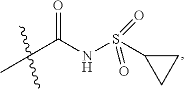 [SMILES: *C(=O)NS(=O)(=O)C1CC1]

substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl), or substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl).

In embodiments, R³ is a substituted (e.g., substituted with at least one substituent group, size-limited substituent group, or lower substituent group) alkyl (e.g., C₁-C₈ alkyl, C₁-C₆ alkyl, or C₁-C₄ alkyl). In embodiments, R³ is an unsubstituted alkyl (e.g., C₁-C₈ alkyl, C₁-C₆ alkyl, or C₁-C₄ alkyl). In embodiments, R³ is a substituted (e.g., substituted with at least one substituent group, size-limited substituent group, or lower substituent group) heteroalkyl (e.g., 2 to 8 membered heteroalkyl, 2 to 6 membered heteroalkyl, or 2 to 4 membered heteroalkyl). In embodiments, R³ is an unsubstituted heteroalkyl (e.g., 2 to 8 membered heteroalkyl, 2 to 6 membered heteroalkyl, or 2 to 4 membered heteroalkyl).

In embodiments, R³ is methyl, ethyl, propyl, butyl, —CH₂OH, —CH₂CH₂OH, —CH₂N₃, —CH₂CH₂N₃, —CH₂OCH₃, —CH₂OCH₂CH₃, —CH₂CH₂OCH₃, —CH₂CH₂OCH₂CH₃,

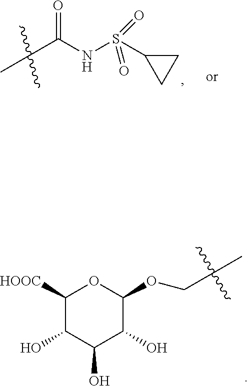 [SMILES: *C(=O)NS(=O)(=O)C1CC1 ; *CO[C@@H]1O[C@H](C(=O)O)[C@@H](O)[C@H](O)[C@H]1O]

In embodiments, R³ is methyl. In embodiments, R³ is ethyl. In embodiments, R³ is propyl. In embodiments, R³ is butyl. In embodiments, R³ is —CH₂OH. In embodiments, R³ is —CH₂ CH₂OH. In embodiments, R³ is —CH₂N₃. In embodiments, R³ is —CH₂CH₂N₃. In embodiments, R³ is —CH₂OCH₃. In embodiments, R³ is —CH₂OCH₂CH₃. In embodiments, R³ is —CH₂CH₂OCH₃. In embodiments, R³ is —CH₂CH₂OCH₂CH₃. In embodiments, R³ is —OH. In embodiments, R³ is H. In embodiments, R³ is

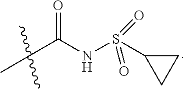 [SMILES: *C(=O)NS(=O)(=O)C1CC1]

In embodiments, R³ is

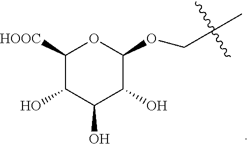 [SMILES: *CO[C@@H]1O[C@H](C(=O)O)[C@@H](O)[C@H](O)[C@H]1O]

In embodiments, R³ is methyl, —CH₂OH,

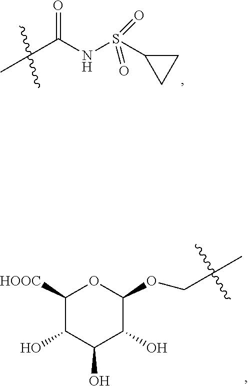 [SMILES: *C(=O)NS(=O)(=O)C1CC1 ; *CO[C@@H]1O[C@H](C(=O)O)[C@@H](O)[C@H](O)[C@H]1O]

or —CH₂N₃.

In embodiments, v is an integer from 1 to 24. In embodiments, v is 1. In embodiments, v is 2. In embodiments, v is 3. In embodiments, v is 4. In embodiments, v is 5. In embodiments, v is 6. In embodiments, v is 7. In embodiments, v is 8. In embodiments, v is 9. In embodiments, v is 10. In embodiments, v is 11. In embodiments, v is 12. In embodiments, v is 13. In embodiments, v is 14. In embodiments, v is 15. In embodiments, v is 16. In embodiments, v is 17. In embodiments, v is 18. In embodiments, v is 19. In embodiments, v is 20. In embodiments, v is 21. In embodiments, v is 22. In embodiments, v is 23. In embodiments, v is 24.

In embodiments, R⁴ is H, halogen, —OR^{4A}, —NR^{4A}R^{4B}, substituted or unsubstituted alkyl (e.g., C₁-C₈ alkyl, C₁-C₆ alkyl, or C₁-C₄ alkyl), or substituted or unsubstituted heteroalkyl (e.g., 2 to 8 membered heteroalkyl, 2 to 6 membered heteroalkyl, or 2 to 4 membered heteroalkyl).

In embodiments, R⁴ is H, —OR^{4A}, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl), or substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl).

In embodiments, R⁴ is a substituted (e.g., substituted with at least one substituent group, size-limited substituent group, or lower substituent group) alkyl (e.g., C₁-C₈ alkyl, C₁-C₆ alkyl, or C₁-C₄ alkyl). In embodiments, R⁴ is an unsubstituted alkyl (e.g., C₁-C₈ alkyl, C₁-C₆ alkyl, or C₁-C₄ alkyl). In embodiments, R⁴ is a substituted (e.g., substituted with at least one substituent group, size-limited substituent group, or lower substituent group) heteroalkyl (e.g., 2 to 8 membered heteroalkyl, 2 to 6 membered heteroalkyl, or 2 to 4 membered heteroalkyl). In embodiments, R⁴ is an unsubstituted heteroalkyl (e.g., 2 to 8 membered heteroalkyl, 2 to 6 membered heteroalkyl, or 2 to 4 membered heteroalkyl).

In embodiments, R⁴ is H, —OH, methyl, ethyl, propyl or butyl. In embodiments, R⁴ is methyl. In embodiments, R⁴ is ethyl. In embodiments, R⁴ is propyl. In embodiments, R⁴ is butyl. In embodiments, R⁴ is H. In embodiments, R⁴ is —OH.

In embodiments, R⁴ is H or —OH.

In embodiments, Z¹ is a substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl). In embodiments, Z¹ is a substituted (e.g. with a substituent group, a size-limited substituent group or a lower substituent group) cycloalkyl (e.g., C₃-C₈ cycloalkyl, C₃-C₆ cycloalkyl, or C₅-C₆ cycloalkyl). In embodiments, Z¹ is an unsubstituted cycloalkyl (e.g., C₃-C₈ cycloalkyl, C₃-C₆ cycloalkyl, or C₅-C₆ cycloalkyl). In embodiments, Z¹ is a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl). In embodiments, Z¹ is a substituted (e.g. with a substituent group, a size-limited substituent group or a lower substituent group) heterocycloalkyl (e.g., 3 to 8 membered heterocycloalkyl, 3 to 6 membered heterocycloalkyl, or 5 to 6 membered heterocycloalkyl). In embodiments, Z¹ is an unsubstituted heterocycloalkyl (e.g., 3 to 8 membered heterocycloalkyl, 3 to 6 membered heterocycloalkyl, or 5 to 6 membered heterocycloalkyl). In embodiments, Z¹ is a substituted (e.g. with a substituent group, a size-limited substituent group or a lower substituent group) or unsubstituted aryl (e.g., C₆-C₁₀ aryl, C₁₀ aryl, or phenyl). In embodiments, Z¹ is a substituted (e.g. with a substituent group, a size-limited substituent group or a lower substituent group) aryl (e.g., C₆-C₁₀ aryl, C₁₀ aryl, or phenyl). In embodiments, Z¹ is an unsubstituted aryl (e.g., C₆-C₁₀ aryl, C₁₀ aryl, or phenyl). In embodiments, Z¹ is a substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, Z¹ is a substituted (e.g. with a substituent group, a size-limited substituent group or a lower substituent group) heteroaryl (e.g., 5 to 10 membered heteroaryl, 5 to 9 membered heteroaryl, or 5 to 6 membered heteroaryl). In embodiments, Z¹ is an unsubstituted heteroaryl (e.g., 5 to 10 membered heteroaryl, 5 to 9 membered heteroaryl, or 5 to 6 membered heteroaryl).

In embodiments, Z¹ is

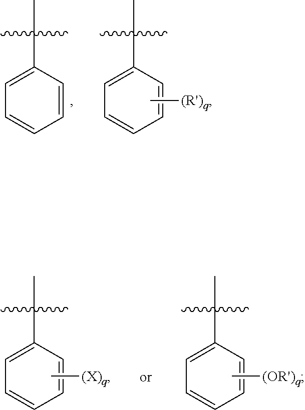

wherein each X is independently Cl, Br, I, or F; each R¹ is independently —CH₃, —CH₂CH₃ or —CH₂CH₂CH₃; and q is an integer from 1 to 5.

In embodiments, q is 1. In embodiments q is 2. In embodiments q is 3. In embodiments q is 4. In embodiments q is 5.

In embodiments, X is Cl. In embodiments, X is Br. In embodiments, X is I. In embodiments, X is F.

In embodiments, R¹ is —CH₃. In embodiments, R¹ is —CH₂CH₃. In embodiments, R¹ is —CH₂CH₂CH₃.

In embodiments, Z¹ is

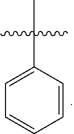 [SMILES: *c1ccccc1]

In embodiments, Z¹ is

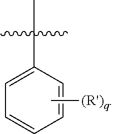

In embodiments, Z¹ is

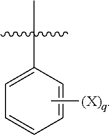

In embodiments, Z¹ is

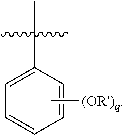

In embodiments, Z² is a substituted (e.g. with a substituent group, a size-limited substituent group or a lower substituent group) or unsubstituted cycloalkylene (e.g., C₃-C₈ cycloalkylene, C₃-C₆ cycloalkylene, or C₅-C₆ cycloalkylene). In embodiments, Z² is a substituted (e.g. with a substituent group, a size-limited substituent group or a lower substituent group) or unsubstituted heterocycloalkylene (e.g., 3 to 8 membered heterocycloalkylene, 3 to 6 membered heterocycloalkylene, or 5 to 6 membered heterocycloalkylene). In embodiments, Z² is a substituted (e.g. with a substituent group, a size-limited substituent group or a lower substituent group) or unsubstituted arylene (e.g., C₆-C₁₀ arylene, C₁₀ arylene, or phenylene). In embodiments, Z2 is a substituted (e.g. with a substituent group, a size-limited substituent group or a lower substituent group) or unsubstituted heteroarylene (e.g., 5 to 10 membered heteroarylene, 5 to 9 membered heteroarylene, or 5 to 6 membered heteroarylene).

In embodiments, Z¹ is an unsubstituted arylene.

In embodiments, Z² is

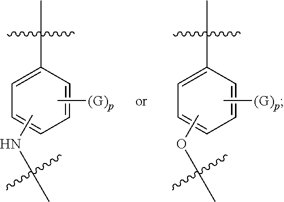

wherein
each G is independently Cl, Br, I, F, —CH₃, —CH₂CH₃, —CH₂CH₂CH₃, —OCH₃, —OCH₂CH₃, —OH, or —NH₂; and p is an integer from 0-4.

In embodiments p is 0. In embodiments p is 1. In embodiments p is 2. In embodiments p is 3. In embodiments p is 4.

In embodiments, G is Cl. In embodiments, G is Br. In embodiments, G is I. In embodiments, G is F. In embodiments, G is —CH₃. In embodiments, G is —CH₂CH₃. In embodiments, G is —CH₂CH₂CH₃. In embodiments, G is —OCH₃. In embodiments, G is —OCH₂CH₃. In embodiments, G is —OH. In embodiments, G is —NH₂.

In embodiments, Z² is

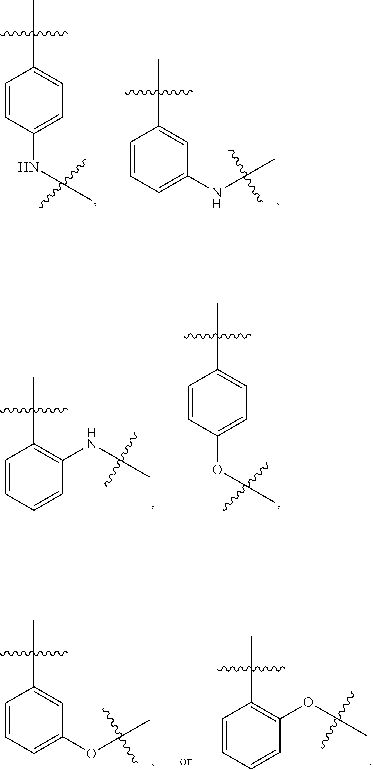 [SMILES: *Nc1ccc(*)cc1 ; *Nc1cccc(*)c1 ; *Nc1ccccc1* ; *Oc1ccc(*)cc1 ; *Oc1cccc(*)c1 ; *Oc1ccccc1*]

In embodiments, Z² is

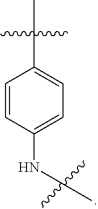 [SMILES: *Nc1ccc(*)cc1]

In embodiments, Z² is

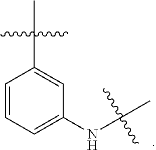 [SMILES: *Nc1cccc(*)c1]

In embodiments, Z² is

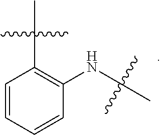 [SMILES: *Nc1ccccc1*]

In embodiments, Z² is

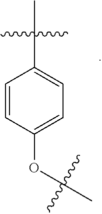 [SMILES: *Oc1ccc(*)cc1]

In embodiments, Z² is

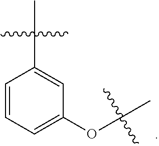 [SMILES: *Oc1cccc(*)c1]

In embodiments, Z² is

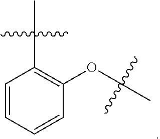 [SMILES: *Oc1ccccc1*]

In embodiments, R⁶ is H, substituted or unsubstituted alkyl, substituted or unsubstituted cycloalkyl, substituted or unsubstituted heterocycloalkyl, substituted or unsubstituted aryl, substituted or unsubstituted heteroaryl, —CO(CH₂CH₂O)_{w}CH₂CH₂Y, —CONH(CH₂CH₂O)_{w}CH₂CH₂Y,

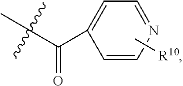

a Charged Group, or a saccharide derivative, w is an integer from 1 to 24; Y is —NH₂, —OH, —COOH, or —OCH₃; R¹⁰ is —OH, —OCH₃ or —COOH.

In embodiments, R⁶ is H or substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl), substituted (e.g., substituted with at least one substituent group, size-limited substituent group, or lower substituent group) or unsubstituted cycloalkyl (e.g., C₃-C₈ cycloalkyl, C₃-C₆ cycloalkyl, or C₅-C₆ cycloalkyl), substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with at least one substituent group, size-limited substituent group, or lower substituent group) or unsubstituted aryl (e.g., C₆-C₁₀ aryl, C₁₀ aryl, or phenyl), substituted (e.g., substituted with at least one substituent group, size-limited substituent group, or lower substituent group) or unsubstituted heteroaryl (e.g., 5 to 10 membered heteroaryl, 5 to 9 membered heteroaryl, or 5 to 6 membered heteroaryl), or a saccharide derivative.

In embodiments, R⁶ is H, a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl). In embodiments, R⁶ is a substituted (e.g. with a substituent group, a size-limited substituent group or a lower substituent group) heterocycloalkyl (e.g., 3 to 8 membered heterocycloalkyl, 3 to 6 membered heterocycloalkyl, or 5 to 6 membered heterocycloalkyl). In embodiments, R⁶ is an unsubstituted heterocycloalkyl (e.g., 3 to 8 membered heterocycloalkyl, 3 to 6 membered heterocycloalkyl, or 5 to 6 membered heterocycloalkyl).

In embodiments, R⁶ is H or substituted (e.g. with a substituent group, a size-limited substituent group or a lower substituent group) heterocycloalkyl (e.g., 3 to 8 membered heterocycloalkyl, 3 to 6 membered heterocycloalkyl, or 5 to 6 membered heterocycloalkyl).

In embodiments, R⁶ is H or

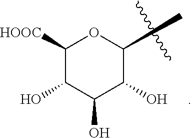

In embodiments, R⁶ is —CO(CH₂CH₂O)_{w}CH₂CH₂Y or —CONH(CH₂CH₂O)_{w}CH₂CH₂Y, where w is an integer from 1 to 24 and Y is —NH₂, —OH, —COOH, or —OCH₃. In embodiments, R⁶ is —CO(CH₂CH₂O)_{w}CH₂CH₂NH₂. In embodiments, R⁶ is —CO(CH₂CH₂O)_{w}CH₂CH₂OH. In embodiments, R⁶ is —CO(CH₂CH₂O)_{w}CH₂CH₂COOH. In embodiments, R⁶ is —CO(CH₂CH₂O)_{w}CH₂CH₂OCH₃. In embodiments, R⁶ is —CONH(CH₂CH₂O)_{w}CH₂CH₂NH₂. In embodiments, R⁶ is —CONH(CH₂CH₂O)_{w}CH₂CH₂OH. In embodiments, R⁶ is —CONH(CH₂CH₂O)_{w}CH₂CH₂COOH. In embodiments, R⁶ is —CONH(CH₂CH₂O)_{w}CH₂CH₂OCH₃.

In embodiments, w is an integer from 1 to 24. In embodiments, w is 1. In embodiments, w is 2. In embodiments, w is 3. In embodiments, w is 4. In embodiments, w is 5. In embodiments, w is 6. In embodiments, w is 7. In embodiments, w is 8. In embodiments, w is 9. In embodiments, w is 10. In embodiments, w is 11. In embodiments, w is 12. In embodiments, w is 13. In embodiments, w is 14. In embodiments, w is 15. In embodiments, w is 16. In embodiments, w is 17. In embodiments, w is 18. In embodiments, w is 19. In embodiments, w is 20. In embodiments, w is 21. In embodiments, w is 22. In embodiments, w is 23. In embodiments, w is 24.

In embodiments, Y is —NH₂, —OH, —COOH, or —OCH₃ In embodiments, Y is —NH₂. In embodiments, Y is —OH. In embodiments, Y is —COOH. In embodiments, Y is —OCH₃.

In embodiments, R⁶ is

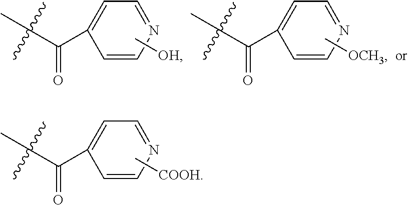

In embodiments, R⁶ is

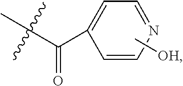

In embodiments, R⁶ is

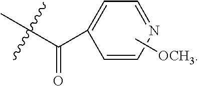

In embodiments, R⁶ is

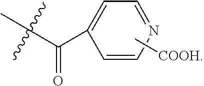

In embodiments, R⁶ is a saccharide derivative. In embodiments, R⁶ is

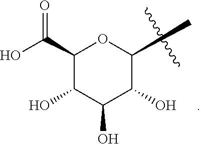

In embodiments, R⁶ is

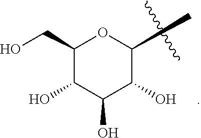

In embodiments, R⁶ is

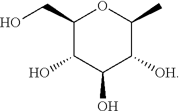

In embodiments, each R^{3A}, R^{3B}, R^{4A}, and R^{4B} is independently H or substituted or unsubstituted alkyl (e.g., C₁-C₈ alkyl, C₁-C₆ alkyl, or C₁-C₄ alkyl).

In embodiments, each R^{3A}, R^{3B}, R^{4A}, and R^{4B} is independently H or substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl). In embodiments, each R^{3A}, R^{3B}, R^{4A}, and R^{4B} is independently H. In embodiments, each R^{3A}, R^{3B}, R^{3A}, and R^{4B} is independently substituted (e.g., substituted with at least one substituent group, size-limited substituent group, or lower substituent group) alkyl (e.g., C₁-C₈ alkyl, C₁-C₆ alkyl, or C₁-C₄ alkyl). In embodiments, each R^{3A}, R^{3B}, R^{4A}, and R^{4B} is independently unsubstituted alkyl (e.g., C₁-C₈ alkyl, C₁-C₆ alkyl, or C₁-C₄ alkyl).

In embodiments, each R^{3A}, R^{3B}, R^{4A}, and R^{4B} is independently H, methyl, ethyl, propyl, isopropyl, butyl, isobutyl, tert-butyl, or pentyl. In embodiments, each R^{3A}, R^{3B}, R^{4A}, and R^{4B} is independently H. In embodiments, each R^{3A}, R^{3B}, R^{4A}, and R^{4B} is independently methyl. In embodiments, each R^{3A}, R^{3B}, R^{4A}, and R^{4B} is independently ethyl. In embodiments, each R^{3A}, R^{3B}, R^{4A}, and R^{4B} is independently propyl. In embodiments, each R^{3A}, R^{3B}, R^{4A}, and R^{4B} is independently isopropyl. In embodiments, each R^{3A}, R^{3B}, R^{4A}, and R^{4B} is independently butyl. In embodiments, each R^{3A}, R^{3B}, R^{4A}, and R^{4B} is independently isobutyl. In embodiments, each R^{3A}, R^{3B}, R^{4A}, and R^{4B} is independently tert-butyl. In embodiments, each R^{3A}, R^{3B}, R^{4A}, and R^{4B} is independently pentyl.

In embodiments, D is:

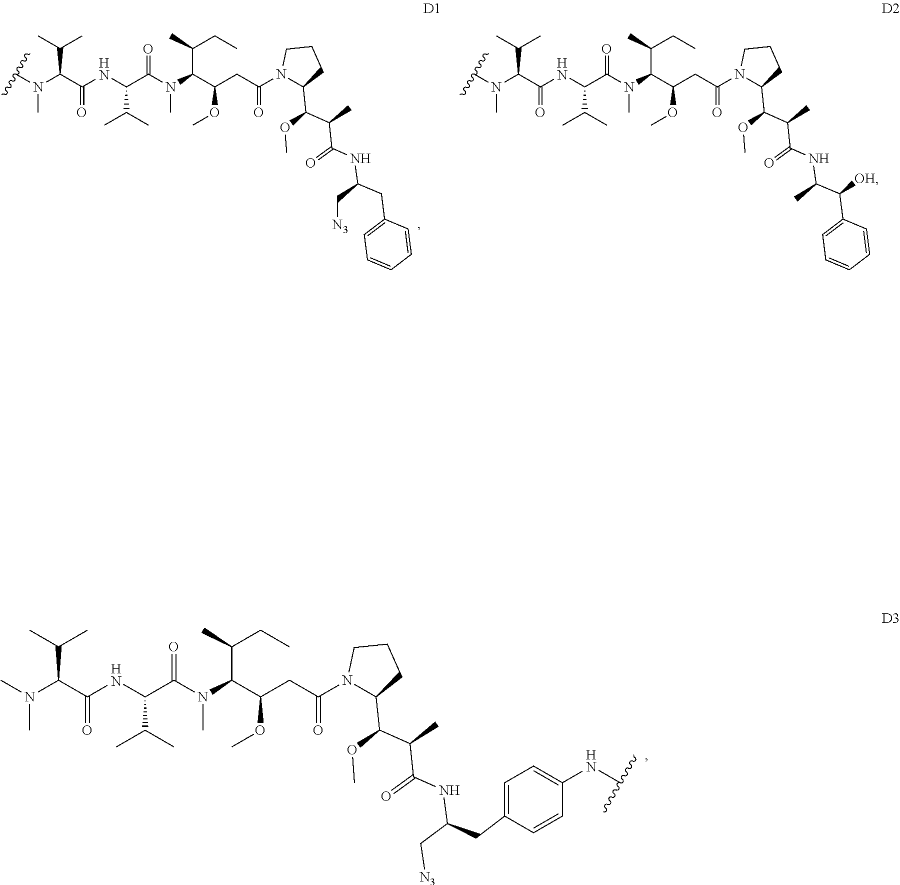 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](CN=[N+]=[N-])Cc1ccccc1)OC)C(C)C)C(C)C ; *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C ; *Nc1ccc(C[C@@H](CN=[N+]=[N-])NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)cc1]

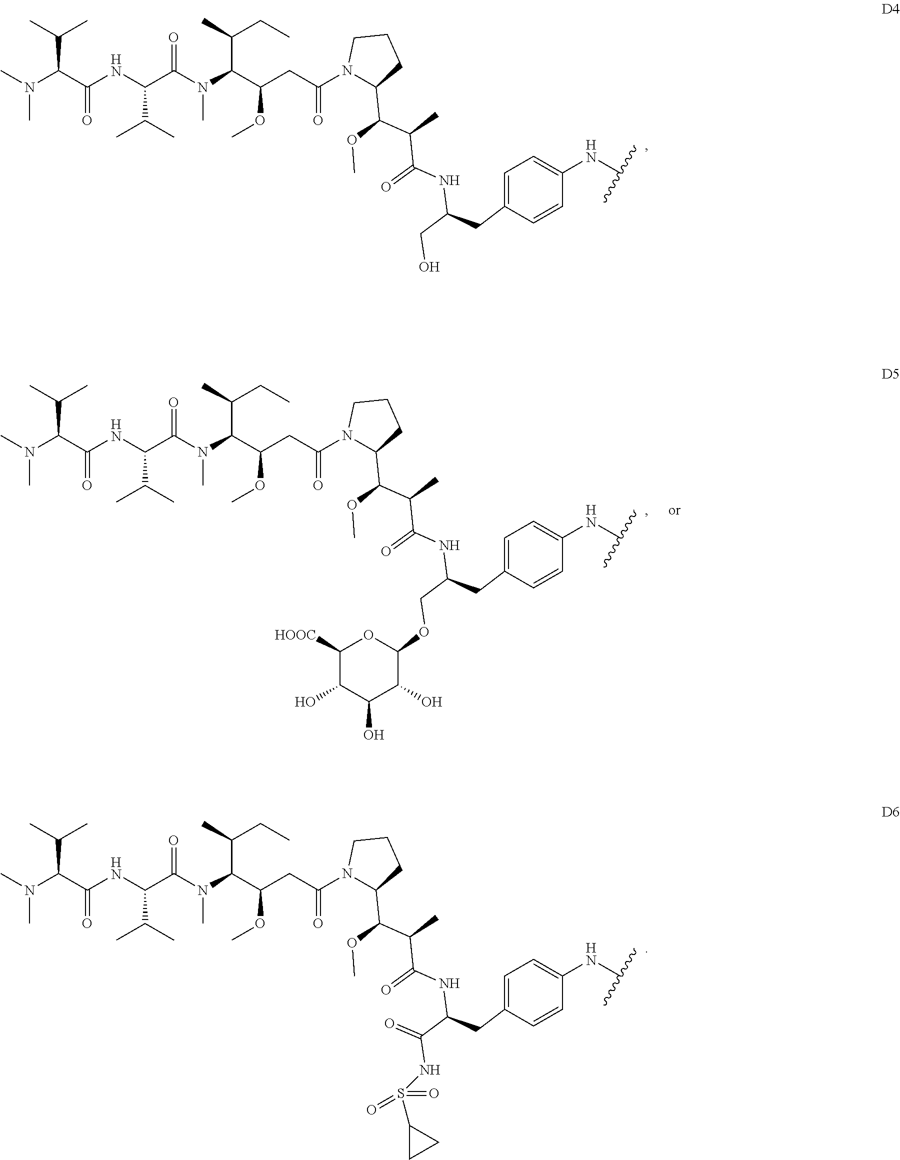 [SMILES: *Nc1ccc(C[C@@H](CO)NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)cc1 ; *Nc1ccc(C[C@@H](CO[C@@H]2O[C@H](C(=O)O)[C@@H](O)[C@H](O)[C@H]2O)NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)cc1 ; *Nc1ccc(C[C@H](NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)C(=O)NS(=O)(=O)C2CC2)cc1]

In embodiments, D is:

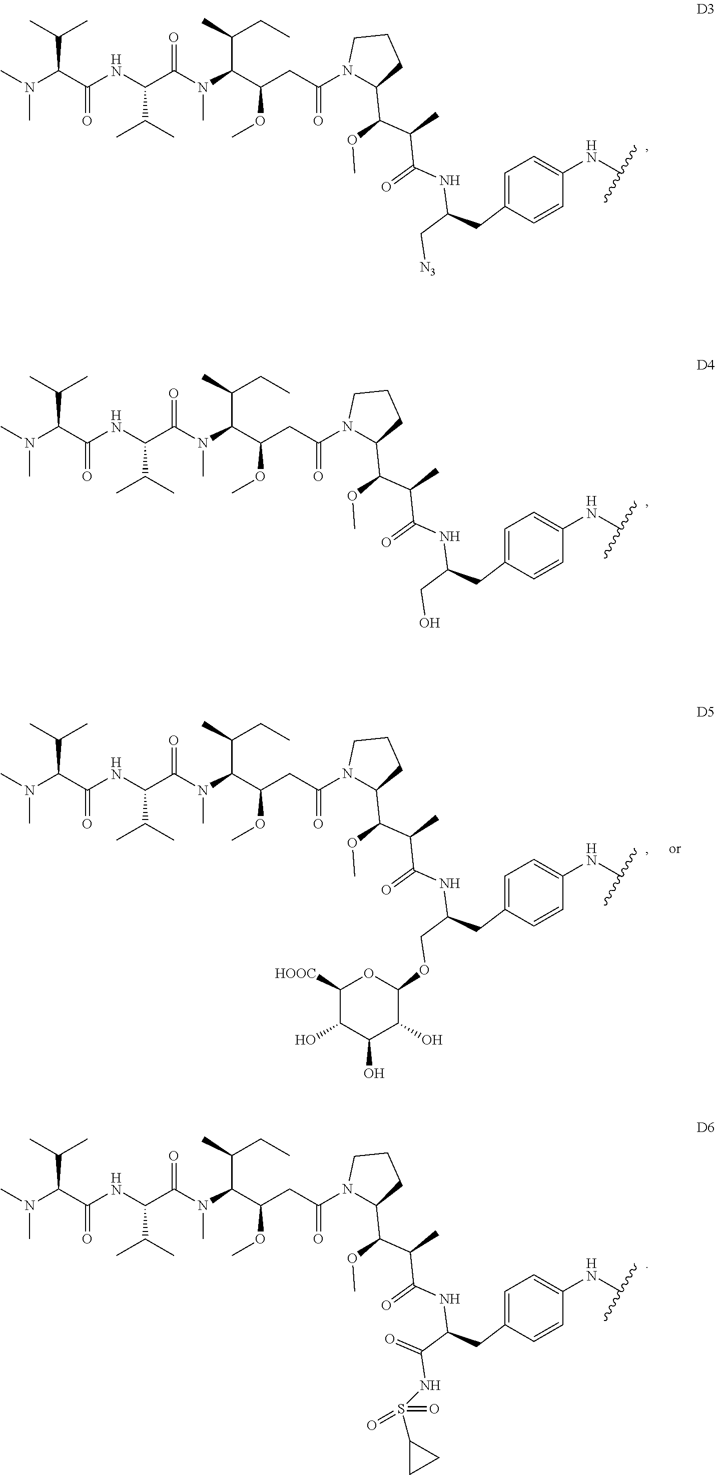

In embodiments, D is:

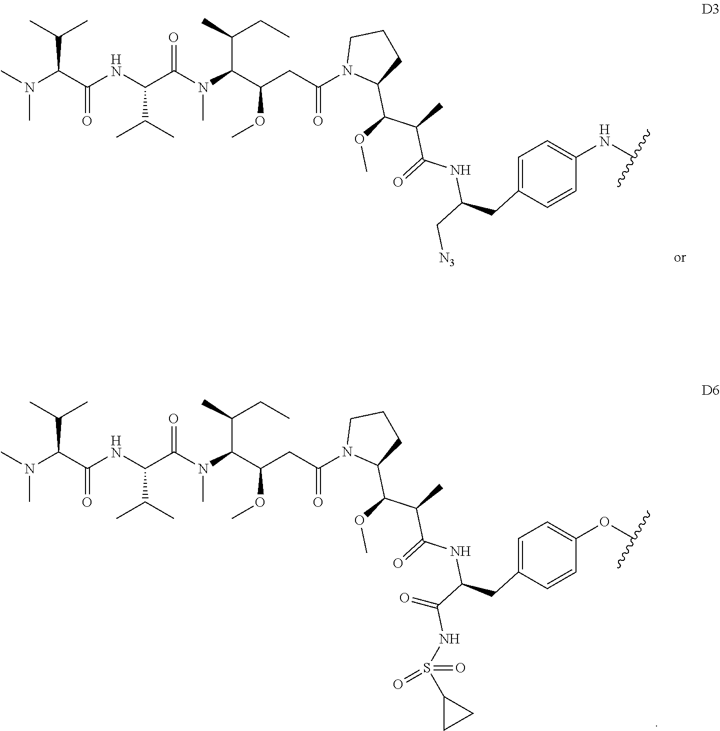 [SMILES: *Nc1ccc(C[C@@H](CN=[N+]=[N-])NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)cc1 ; *Oc1ccc(C[C@H](NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)C(=O)NS(=O)(=O)C2CC2)cc1]

In embodiments, D is:

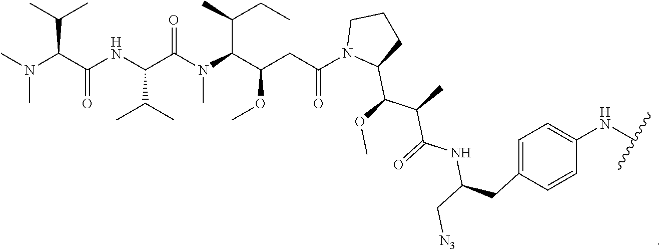 [SMILES: *Nc1ccc(C[C@@H](CN=[N+]=[N-])NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)cc1]

In embodiments, D is:

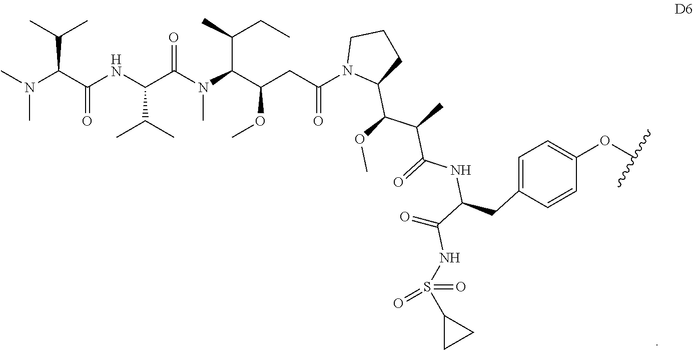 [SMILES: *Oc1ccc(C[C@H](NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)C(=O)NS(=O)(=O)C2CC2)cc1]

In embodiments, D is:

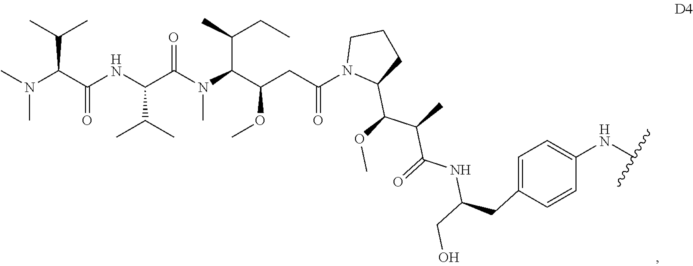 [SMILES: *Nc1ccc(C[C@@H](CO)NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)cc1]

In embodiments, D is:

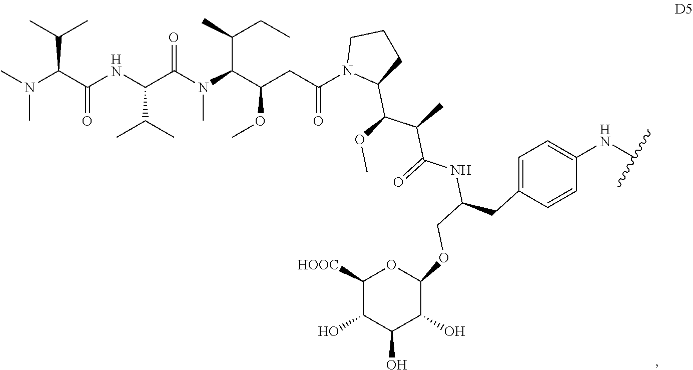 [SMILES: *Nc1ccc(C[C@@H](CO[C@@H]2O[C@H](C(=O)O)[C@@H](O)[C@H](O)[C@H]2O)NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)cc1]

In embodiments, the anti-BCMA ADC is:

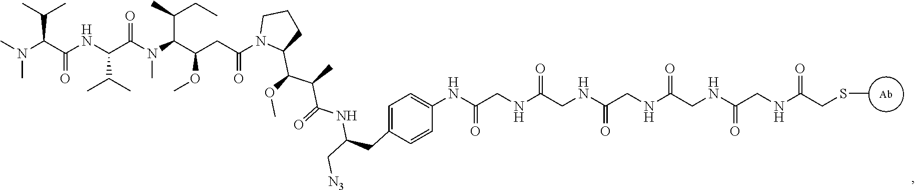

### ADC-1 (Compound 1 Conjugated with Anti-BCMA Antibody; DAR 3.8)

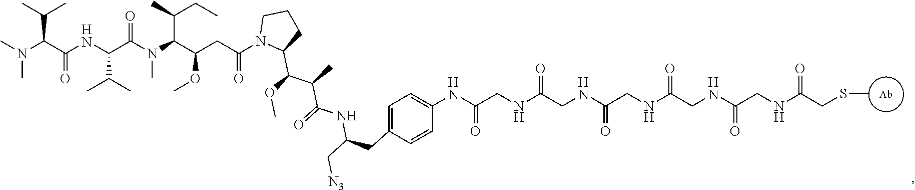

### ADC-2 (Compound 2 Conjugated with Anti-BCMA Antibody; DAR 3.4)

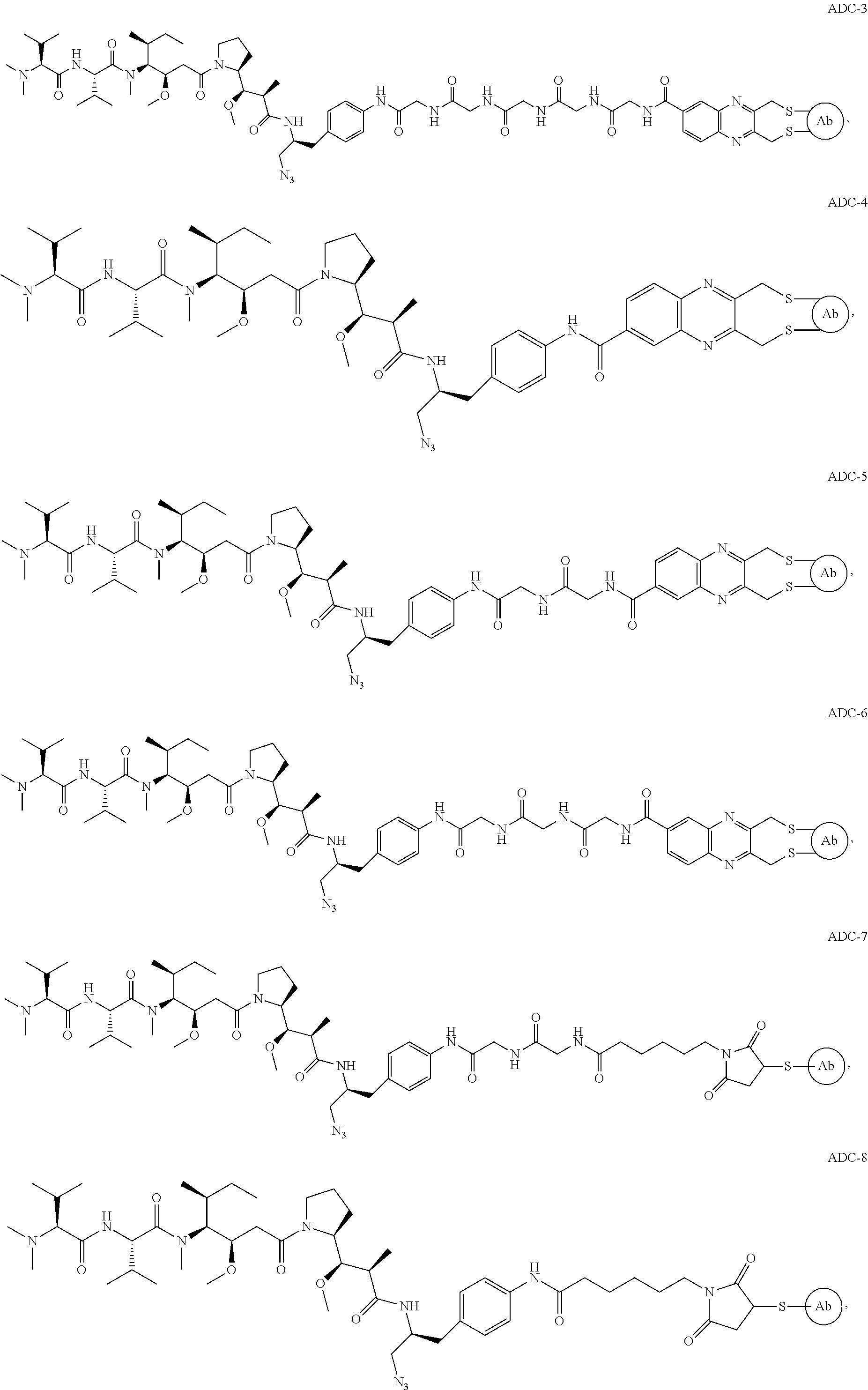

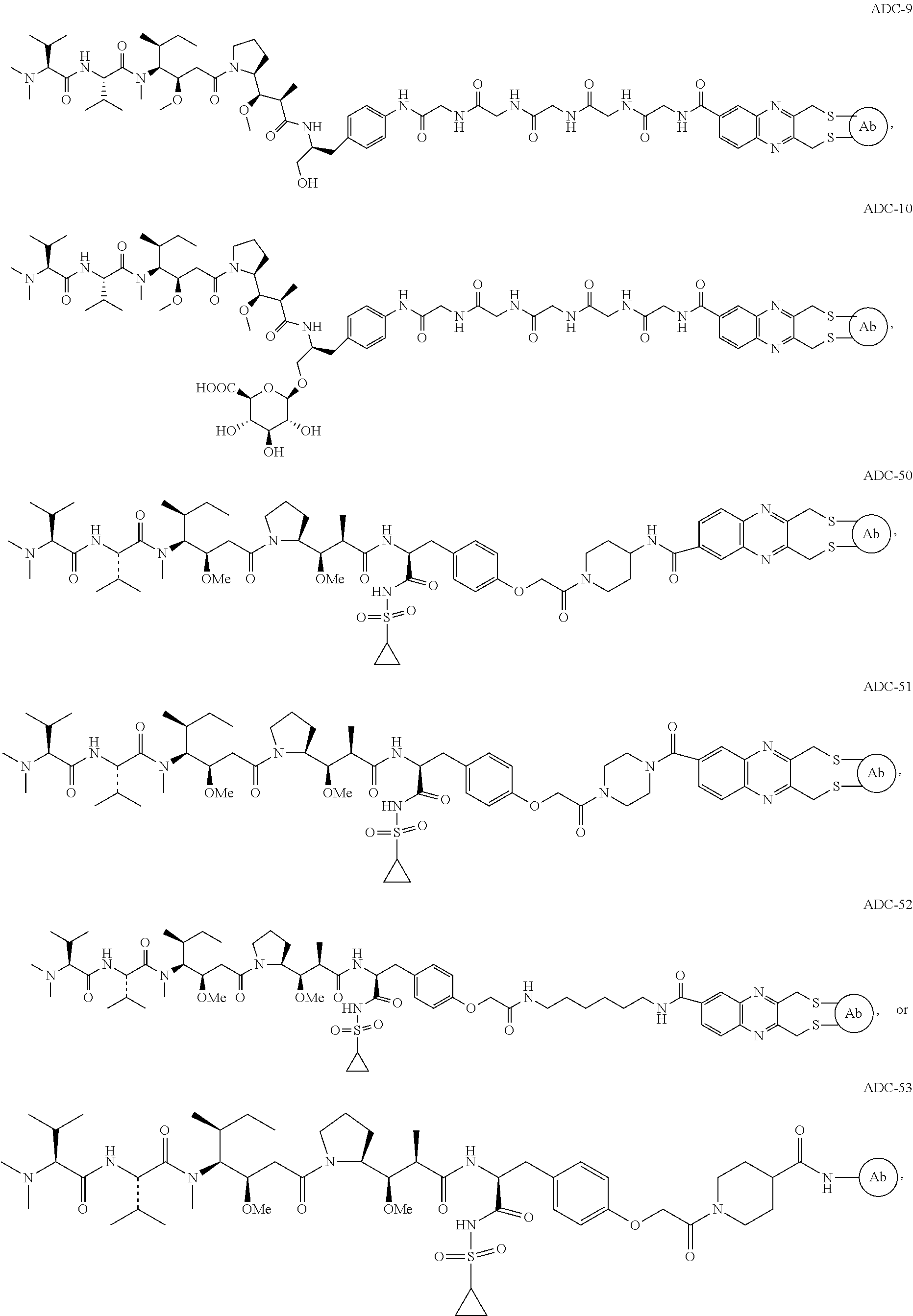

or a pharmaceutically acceptable salt thereof.

Precursors

In an aspect, provided herein is a compound of formula (II):

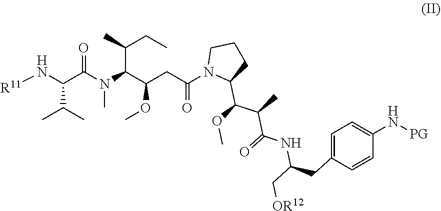

or a pharmaceutically acceptable salt thereof, wherein:
PG is an amine protecting group:
R¹¹ is H or one or more Amino Acid Units; and
R¹² is H or a substituted alkyl, substituted heteroalkyl, substituted heterocycloalkyl, —CO(CH₂CH₂O)ₛCH₂CH₂U, or —CONH(CH₂CH₂O)ₛCH₂CH₂U; wherein
s is an integer from 1 to 24; and U is —NH₂, —OH, —COOH, or —OCH₃.

In embodiments, R¹² is a substituted (e.g., substituted with at least one substituent group, size-limited substituent group, or lower substituent group) alkyl (e.g., C₁-C₈ alkyl, C₁-C₆ alkyl, or C₁-C₄ alkyl), substituted (e.g., substituted with at least one substituent group, size-limited substituent group, or lower substituent group) heterocycloalkyl (e.g., 3 to 8 membered heterocycloalkyl, 3 to 6 membered heterocycloalkyl, or 5 to 6 membered heterocycloalkyl), substituted (e.g., substituted with at least one substituent group, size-limited substituent group, or lower substituent group) or unsubstituted aryl (e.g., C₆-C₁₀ aryl, C₁₀ aryl, or phenyl), substituted (e.g., substituted with at least one substituent group, size-limited substituent group, or lower substituent group) or unsubstituted heteroaryl (e.g., 5 to 10 membered heteroaryl, 5 to 9 membered heteroaryl, or 5 to 6 membered heteroaryl)

In embodiments, R¹² is H or substituted (e.g. with a substituent group, a size-limited substituent group or a lower substituent group) heterocycloalkyl (e.g., 3 to 8 membered heterocycloalkyl, 3 to 6 membered heterocycloalkyl, or 5 to 6 membered heterocycloalkyl). In embodiments, R¹² is substituted (e.g. with a substituent group, a size-limited substituent group or a lower substituent group) heterocycloalkyl (e.g., 3 to 8 membered heterocycloalkyl, 3 to 6 membered heterocycloalkyl, or 5 to 6 membered heterocycloalkyl).

In embodiments, R¹² is H or

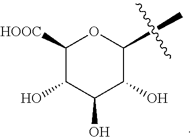

In embodiments, R¹² is H. In embodiments, R¹² is

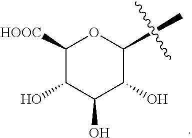

In embodiments, R¹¹ is H or one Amino Acid Unit. In embodiments, R¹¹ is H. In embodiments, R¹¹ is two Amino Acid Units. In embodiments, R¹¹ is three Amino Acid Units. In embodiments, R¹¹ is four Amino Acid Units. In embodiments, R¹¹ is five Amino Acid Units.

In embodiments, R¹¹ is H or one or more hydrophobic amino acid. In embodiments, R¹¹ is one hydrophobic amino acid. In embodiments, R¹¹ is two hydrophobic amino acids. In embodiments, R¹¹ is three hydrophobic amino acids. In embodiments, R¹¹ is four hydrophobic amino acids. In embodiments, R¹¹ is five hydrophobic amino acids. In embodiments, R¹¹ is H.

In embodiments, R¹¹ is one or more of valine, isoleucine, leucine, methionine, phenylalanine, alanine, L-norleucine, proline, tryptophan, 2-aminoisobutyric acid, or 3-cyclohexyl-L-alanine. In embodiments, R¹¹ is valine. In embodiments, R¹¹ is isoleucine. In embodiments, R¹¹ is leucine. In embodiments, R¹¹ is methionine. In embodiments, R¹¹ is phenylalanine. In embodiments, R¹¹ is alanine. In embodiments, R¹¹ is L-norleucine. In embodiments, R¹¹ is proline. In embodiments, R¹¹ is tryptophan. In embodiments, R¹¹ is 2-aminoisobutyric acid. In embodiments, R¹¹ is 3-cyclohexyl-L-alanine.

In embodiments, PG is Boc, Fmoc, or CBZ. In embodiments, PG is Boc. In embodiments, PG is Fmoc. In embodiments, PG is CBZ.

In embodiments, the compound of formula (II) is:

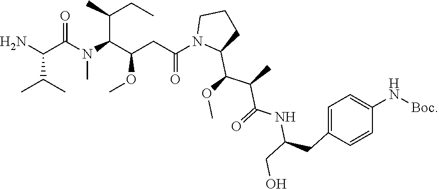 [SMILES: CC[C@H](C)[C@@H]([C@@H](CC(=O)N1CCC[C@H]1[C@H](OC)[C@@H](C)C(=O)N[C@H](CO)Cc1ccc(NC(=O)OC(C)(C)C)cc1)OC)N(C)C(=O)[C@@H](N)C(C)C]

Drug Loading

Drug loading is represented by m, the average number of drug moieties (i.e., D) per monoclonal antibody in an antibody drug conjugate (ADC) of formula (I) and variations thereof. Drug loading may range from 1 to 20 drug moieties per antibody. The ADCs of formula (I), and any embodiment, variation, or aspect thereof, include collections of antibodies conjugated with a range of drug moieties, from 1 to 20. The average number of drug moieties per antibody in preparations of ADCs from conjugation reactions may be characterized by conventional means such as mass spectroscopy, ELISA assay, and HPLC. The quantitative distribution of ADCs in terms of m may also be determined. In some instances, separation, purification, and characterization of homogeneous ADCs where m is a certain value from ADCs with other drug loadings may be achieved by means such as reverse phase HPLC or electrophoresis. In embodiments, the monoclonal antibody is an anti-BCMA, anti-ROR1, anti-CD25, or anti-Claudine 18 antibody. In embodiments, the average number of drug moieties (i.e. D) per anti-BCMA antibody may range from 1 to 20 drug moieties per antibody.

For some ADCs, m may be limited by the number of attachment sites on the antibody. For example, where the attachment is a cysteine thiol, as in some of the exemplary embodiments described herein, an antibody may have only one or several cysteine thiol groups, or may have only one or several sufficiently reactive thiol groups through which a linker may be attached. In embodiments, the average drug loading for ADC ranges from 1 to about 8, or from about 3 to about 8. In embodiments, L¹ is capable of forming a covalent bond with the thiol groups of the free cysteine(s) in the IgG antibody.

Conjugation methods to derivatize a polypeptide with a payload can be accomplished by forming an amide bond with a lysine side chain. Due to the presence of large number of lysine side chain amines with similar reactivity, this conjugation strategy can produce very complex heterogeneous mixtures. The compositions and methods provided herein provide conjugation through lysine, where, in some embodiments, enhanced selectivity of the lysine can result in a less heterogenous mixture. In embodiments, the average drug loading for ADC ranges from 1 to about 20, from 1 to about 8, or from about 3 to about 8. In embodiments, L¹ is capable of forming a covalent bond with the amine group(s) of the lysine(s) in the IgG antibody.

In embodiments, fewer than the theoretical maximum of drug moieties are conjugated to an antibody during a conjugation reaction. Generally, antibodies do not contain many free and reactive cysteine thiol groups which may be linked to a drug moiety; indeed, most cysteine thiol residues in antibodies exist as disulfide bridges. In embodiments, an antibody may be reduced with a reducing agent such as dithiothreitol (DTT) or tricarbonylethylphosphine (TCEP), under partial or total reducing conditions, to generate reactive cysteine thiol groups. In embodiments, an antibody is subjected to denaturing conditions to reveal reactive nucleophilic groups such as lysine or cysteine.

The loading (drug/antibody ratio or “DAR”) of an ADC may be controlled in different ways, and for example, by: (i) limiting the molar excess of drug-linker intermediate or linker reagent relative to antibody, (ii) limiting the conjugation reaction time or temperature, and (iii) partial or limiting reductive conditions for cysteine thiol modification. DAR can also be controlled by the reactivity of the groups reacting with the antibody (e.g., Compound 1 and Compound 2 yield the same ADC structure, but because the reactivity of Compound 1 is greater than that of Compound 2, the DAR of ADC-1 is greater than DAR of ADC-2 and thus the EC50s and in vivo activity of the two ADCs may be different).

It is to be understood that where more than one nucleophilic group reacts with a drug-linker intermediate or linker reagent, then the resulting product is a mixture of ADC compounds with a distribution of one or more drug moieties attached to an antibody. The average number of drugs per antibody may be calculated from the mixture by a dual ELISA antibody assay, which is specific for antibody and specific for the drug. Individual ADC molecules may be identified in the mixture by mass spectroscopy and separated by HPLC, e.g. hydrophobic interaction chromatography (see, e.g., McDonagh et al (2006) Prot. Engr. Design & Selection 19(7):299-307; Hamblett et al (2004) Clin. Cancer Res. 10:7063-7070; Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In embodiments, a homogeneous ADC with a single loading value may be isolated from the conjugation mixture by electrophoresis or chromatography.

Anti-BCMA Antibodies

i. Exemplary Antibodies and Antibody Sequences

In embodiments, the ADC comprises an antibody that binds to BCMA. BCMA has been reported to be upregulated in multiple myeloma independent of baseline levels of BCMA expression. The ADC compounds described herein comprise an anti-BCMA antibody.

In embodiments, the anti-BCMA antibody provided herein comprises a cysteine. In embodiments, the anti-BCMA antibody is bound to a drug through the sulfur of a cysteine residue. In embodiments, the anti-BCMA antibody is bound to a drug through the sulfur of two cysteine residues.

In embodiments, the anti-BCMA antibody provided herein comprises a lysine. In embodiments, the anti-BCMA antibody is bound to a drug through the amine of a lysine residue. In embodiments, the anti-BCMA antibody is bound to a drug through the amine of two lysine residues.

In embodiments, the ADC provided herein comprises an anti-BCMA antibody comprising a light chain variable region and a heavy chain variable region, wherein the light chain variable region comprises a light chain complementarity determining region 1 (CDR1) a light chain CDR2 and a light chain CDR3, and the heavy chain variable region comprises a heavy chain CDR1, a heavy chain CDR2, and a heavy chain CDR3.

In embodiments, the ADC provided herein comprises an anti-BCMA antibody comprising at least one, two, three, four, five, or six CDRs selected from (a) VL CDR1 comprising the sequence of SEQ ID NO: 1; (b) VL CDR2 comprising the sequence of SEQ ID NO: 2; (c) VL CDR3 comprising the sequence of SEQ ID NO: 3; (d) VH CDR1 comprising the sequence of SEQ ID NO: 4; (e) VH CDR2 comprising the sequence of SEQ ID NO: 5; and (f) VH CDR3 comprising the sequence of SEQ ID NO: 6. In embodiments, the ADC comprises an anti-BCMA antibody comprising at least one CDR selected from (a) VL CDR1 comprising the sequence of SEQ ID NO: 1; (b) VL CDR2 comprising the sequence of SEQ ID NO: 2; (c) VL CDR3 comprising the sequence of SEQ ID NO: 3; (d) VH CDR1 comprising the sequence of SEQ ID NO: 4; (e) VH CDR2 comprising the sequence of SEQ ID NO: 5; and (f) VH CDR3 comprising the sequence of SEQ ID NO: 6. In embodiments, the ADC comprises an anti-BCMA antibody comprising at least two CDRs selected from (a) VL CDR1 comprising the sequence of SEQ ID NO: 1; (b) VL CDR2 comprising the sequence of SEQ ID NO: 2; (c) VL CDR3 comprising the sequence of SEQ ID NO: 3; (d) VH CDR1 comprising the sequence of SEQ ID NO: 4; (e) VH CDR2 comprising the sequence of SEQ ID NO: 5; and (f) VH CDR3 comprising the sequence of SEQ ID NO: 6. In embodiments, the ADC comprises an anti-BCMA antibody comprising at least three CDRs selected from (a) VL CDR1 comprising the sequence of SEQ ID NO: 1; (b) VL CDR2 comprising the sequence of SEQ ID NO: 2; (c) VL CDR3 comprising the sequence of SEQ ID NO: 3; (d) VH CDR1 comprising the sequence of SEQ ID NO: 4; (e) VH CDR2 comprising the sequence of SEQ ID NO: 5; and (f) VH CDR3 comprising the sequence of SEQ ID NO: 6. In embodiments, the ADC comprises an anti-BCMA antibody comprising at least four CDRs selected from (a) VL CDR1 comprising the sequence of SEQ ID NO: 1; (b) VL CDR2 comprising the sequence of SEQ ID NO: 2; (c) VL CDR3 comprising the sequence of SEQ ID NO: 3; (d) VH CDR1 comprising the sequence of SEQ ID NO: 4; (e) VH CDR2 comprising the sequence of SEQ ID NO: 5; and (f) VH CDR3 comprising the sequence of SEQ ID NO: 6. In embodiments, the ADC comprises an anti-BCMA antibody comprising at least five CDRs selected from (a) VL CDR1 comprising the sequence of SEQ ID NO: 1; (b) VL CDR2 comprising the sequence of SEQ ID NO: 2; (c) VL CDR3 comprising the sequence of SEQ ID NO: 3; (d) VH CDR1 comprising the sequence of SEQ ID NO: 4; (e) VH CDR2 comprising the sequence of SEQ ID NO: 5; and (f) VH CDR3 comprising the sequence of SEQ ID NO: 6. In embodiments, the ADC comprises an anti-BCMA antibody comprising at least six CDRs selected from (a) VL CDR1 comprising the sequence of SEQ ID NO: 1; (b) VL CDR2 comprising the sequence of SEQ ID NO: 2; (c) VL CDR3 comprising the sequence of SEQ ID NO: 3; (d) VH CDR1 comprising the sequence of SEQ ID NO: 4; (e) VH CDR2 comprising the sequence of SEQ ID NO: 5; and (f) VH CDR3 comprising the sequence of SEQ ID NO: 6.

In embodiments, the ADC comprises an anti-BCMA antibody comprising one CDR selected from (a) VL CDR1 comprising the sequence of SEQ ID NO: 1; (b) VL CDR2 comprising the sequence of SEQ ID NO: 2; (c) VL CDR3 comprising the sequence of SEQ ID NO: 3; (d) VH CDR1 comprising the sequence of SEQ ID NO: 4; (e) VH CDR2 comprising the sequence of SEQ ID NO: 5; and (f) VH CDR3 comprising the sequence of SEQ ID NO: 6. In embodiments, the ADC comprises an anti-BCMA antibody comprising two CDRs selected from (a) VL CDR1 comprising the sequence of SEQ ID NO: 1; (b) VL CDR2 comprising the sequence of SEQ ID NO: 2; (c) VL CDR3 comprising the sequence of SEQ ID NO: 3; (d) VH CDR1 comprising the sequence of SEQ ID NO: 4; (e) VH CDR2 comprising the sequence of SEQ ID NO: 5; and (f) VH CDR3 comprising the sequence of SEQ ID NO: 6. In embodiments, the ADC comprises an anti-BCMA antibody comprising three CDRs selected from (a) VL CDR1 comprising the sequence of SEQ ID NO: 1; (b) VL CDR2 comprising the sequence of SEQ ID NO: 2; (c) VL CDR3 comprising the sequence of SEQ ID NO: 3; (d) VH CDR1 comprising the sequence of SEQ ID NO: 4; (e) VH CDR2 comprising the sequence of SEQ ID NO: 5; and (f) VH CDR3 comprising the sequence of SEQ ID NO: 6. In embodiments, the ADC comprises an anti-BCMA antibody comprising four CDRs selected from (a) VL CDR1 comprising the sequence of SEQ ID NO: 1; (b) VL CDR2 comprising the sequence of SEQ ID NO: 2; (c) VL CDR3 comprising the sequence of SEQ ID NO: 3; (d) VH CDR1 comprising the sequence of SEQ ID NO: 4; (e) VH CDR2 comprising the sequence of SEQ ID NO: 5; and (f) VH CDR3 comprising the sequence of SEQ ID NO: 6. In embodiments, the ADC comprises an anti-BCMA antibody comprising five CDRs selected from (a) VL CDR1 comprising the sequence of SEQ ID NO: 1; (b) VL CDR2 comprising the sequence of SEQ ID NO: 2; (c) VL CDR3 comprising the sequence of SEQ ID NO: 3; (d) VH CDR1 comprising the sequence of SEQ ID NO: 4; (e) VH CDR2 comprising the sequence of SEQ ID NO: 5; and (f) VH CDR3 comprising the sequence of SEQ ID NO: 6. In embodiments, the ADC comprises an anti-BCMA antibody comprising six CDRs selected from (a) VL CDR1 comprising the sequence of SEQ ID NO: 1; (b) VL CDR2 comprising the sequence of SEQ ID NO: 2; (c) VL CDR3 comprising the sequence of SEQ ID NO: 3; (d) VH CDR1 comprising the sequence of SEQ ID NO: 4; (e) VH CDR2 comprising the sequence of SEQ ID NO: 5; and (f) VH CDR3 comprising the sequence of SEQ ID NO: 6.

In embodiments, the anti-BCMA antibody comprises a VL CDR1 comprising the sequence of SEQ ID NO: 1, a VL CDR2 comprising the sequence of SEQ ID NO: 2, a VL CDR3 comprising the sequence of SEQ ID NO: 3, a VH CDR1 comprising the sequence of SEQ ID NO: 4, a VH CDR2 comprising the sequence of SEQ ID NO: 5, and a VH CDR3 comprising the sequence of SEQ ID NO: 6. In embodiments, the anti-BCMA antibody comprises a VL CDR1 comprising the sequence of SEQ ID NO: 1. In embodiments, the anti-BCMA antibody comprises a VL CDR2 comprising the sequence of SEQ ID NO: 2. In embodiments, the anti-BCMA antibody comprises a VL CDR3 comprising the sequence of SEQ ID NO: 3. In embodiments, the anti-BCMA antibody comprises a VH CDR1 comprising the sequence of SEQ ID NO: 4. In embodiments, the anti-BCMA antibody comprises a VH CDR2 comprising the sequence of SEQ ID NO: 5. In embodiments, the anti-BCMA antibody comprises and a VH CDR3 comprising the sequence of SEQ ID NO: 6.

In embodiments, the ADC comprises an anti-BCMA antibody comprising (a) the light chain CDR1 has the amino acid sequence of SEQ ID NO:1, the light chain CDR2 has the amino acid sequence of SEQ ID NO:2, the light chain CDR3 has the amino acid sequence of SEQ ID NO:3, the heavy chain CDR1 has the amino acid sequence of SEQ ID NO:4, the heavy chain CDR2 has the amino acid sequence of SEQ ID NO:5, and the heavy chain CDR3 has the amino acid sequence of SEQ ID NO:6; or (b) the light chain CDR1 has the amino acid sequence of SEQ ID NO:9, the light chain CDR2 has the amino acid sequence of SEQ ID NO:10, the light chain CDR3 has the amino acid sequence of SEQ ID NO:11, the heavy chain CDR1 has the amino acid sequence of SEQ ID NO:12, the heavy chain CDR2 has the amino acid sequence of SEQ ID NO:13, and the heavy chain CDR3 has the amino acid sequence of SEQ ID NO:14.

In embodiments, the anti-BCMA antibody comprises a VL having a sequence with at least 95%, 96%, 97%, 98%, or 99% identity to SEQ ID NO: 7 or 15. In embodiments, the anti-BCMA antibody comprises a VL having the sequence of SEQ ID NO: 7 or 15. In embodiments, a VL sequence having at least 95%, 96%, 97%, 98%, or 99⁰% identity to SEQ ID NO: 7 or 15 contains substitutions (e.g., conservative substitutions), insertions, or deletions relative to the reference sequence, but an anti-BCMA antibody comprising that sequence retains the ability to bind to BCMA. In embodiments, a total of 1 to 10 amino acids have been substituted, inserted and/or deleted in SEQ ID NO: 7 or 15. In embodiments, a total of 1 to 5 amino acids have been substituted, inserted and/or deleted in SEQ ID NO: 7 or 15. In embodiments, substitutions, insertions, or deletions occur in regions outside the CDRs (i.e., in the FRs). In embodiments, the anti-BCMA antibody comprises the VL sequence of SEQ ID NO: 7 or 15, and includes post-translational modifications of that sequence.

In embodiments, the anti-BCMA antibody comprises a VH having a sequence with at least 95%, 96%, 97%, 98%, or 99% identity to SEQ ID NO: 8. In embodiments, the anti-BCMA antibody comprises a VH having the sequence of SEQ ID NO: 8. In embodiments, a VH sequence having at least 95%, 96%, 97%, 98%, or 99% identity to SEQ ID NO: 8 contains substitutions (e.g., conservative substitutions), insertions, or deletions relative to the reference sequence, but an anti-BCMA antibody comprising that sequence retains the ability to bind to BCMA. In embodiments, a total of 1 to 10 amino acids have been substituted, inserted and/or deleted in SEQ ID NO: 8. In embodiments, a total of 1 to 5 amino acids have been substituted, inserted and/or deleted in SEQ ID NO: 8. In embodiments, substitutions, insertions, or deletions occur in regions outside the CDRs (i.e., in the FRs). In embodiments, the anti-BCMA antibody comprises the VH sequence of SEQ ID NO: 8, and includes post-translational modifications of that sequence.

In embodiments, the anti-BCMA antibody is an IgG antibody. In embodiments, the anti-BCMA antibody is an IgG1, IgG2, IgG3 or IgG4 antibody. In embodiments, the anti-BCMA antibody is an IgG1 or IgG4 antibody. In embodiments, the anti-BCMA antibody is an IgG1 antibody.

In embodiments, an anti-BCMA antibody binds a human BCMA. In embodiments, the human BCMA has the amino acid sequence of SEQ ID NO: 16.

In any of the above embodiments, an anti-BCMA antibody is humanized. In embodiment, an anti-BCMA antibody comprises CDRs as in any of the above embodiments, and further comprises a human acceptor framework, e.g. a human immunoglobulin framework or a human consensus framework. In embodiments, a humanized anti-BCMA antibody comprises (a) a VL CDR1 comprising the sequence of SEQ ID NO: 1; (b) a VL CDR2 comprising the sequence of SEQ ID NO: 2; (c) a VL CDR3 comprising the sequence of SEQ ID NO: 3; (d) a VH CDR1 comprising the sequence of SEQ ID NO: 4; (e) a VH CDR2 comprising the sequence of SEQ ID NO: 5; and (f) a VH CDR3 comprising the sequence of SEQ ID NO: 6. In other embodiments, a humanized anti-BCMA antibody comprises (a) a VL CDR1 comprising the sequence of SEQ ID NO: 9; (b) a VL CDR2 comprising the sequence of SEQ ID NO: 10; (c) a VL CDR3 comprising the sequence of SEQ ID NO: 11; (d) a VH CDR1 comprising the sequence of SEQ ID NO: 12; (e) a VH CDR2 comprising the sequence of SEQ ID NO: 13; and (f) a VH CDR3 comprising the sequence of SEQ ID NO: 14.

In embodiments, the anti-BCMA antibody is a monoclonal antibody, including a chimeric, humanized, or human antibody. In one embodiment, an anti-BCMA antibody is an antibody fragment, e.g., a Fv, Fab, Fab′, scFv, diabody, or F(ab′)₂ fragment. In another embodiment, the antibody is a substantially full-length antibody, e.g., an IgG1 antibody or other antibody class or isotype as defined herein.

ii. Antibody Affinity

In embodiments, an anti-BCMA antibody provided herein binds a human BCMA with an affinity of ≤10 nM, or ≤5 nM, or ≤4 nM, or ≤3 nM, or ≤2 nM. In embodiments, an anti-BCMA antibody binds a human BCMA with an affinity of ≥0.0001 nM, or ≥0.001 nM, or ≥0.01 nM. Standard assays known to the skilled artisan can be used to determine binding affinity. For example, whether an anti-BCMA antibody “binds with an affinity of”≤10 nM, or ≤5 nM, or ≤4 nM, or ≤3 nM, or ≤2 nM, can be determined using standard Scatchard analysis utilizing a non-linear curve fitting program (see, for example, Munson et al., Anal Biochem, 107: 220-239, 1980).

In embodiments, the anti- BCMA antibody provided herein has a dissociation constant (Kd) of ≤1 μM, ≤100 nM, ≤10 nM, ≤1 nM, ≤0.1 nM, ≤0.01 nM, or ≤0.001 nM, and optionally is ≥10⁻¹³ M. (e.g. 10⁻⁸ M or less, e.g. from 10⁻⁸ M to 10⁻¹³ M, e.g., from 10⁻⁹ M to 10⁻¹³ M).

In embodiments, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 μM or 26 μM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., up to about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20@) in PBS. When the plates have dried, 150 μL/well of scintillant (MICROSCINT-20 ™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, NJ) at 25° C. with immobilized antigen CM5 chips at -10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μL/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μL/min. Association rates (kon) and dissociation rates (koff) are calculated using a simple one-to-one Langmuir binding model (BIACORE @ Evaluation Software version 3.2) by simultaneously fitting the association and dissociation sensorgrams. The equilibrium dissociation constant (Kd) is calculated as the ratio koff/kon. See, e.g., Chen et al., J. Mol. Biol. 293:865-881 (1999). If the on-rate exceeds 106 M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

iii. Antibody Fragments

In embodiments, the anti-BCMA antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In embodiments, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

iv. Chimeric and Humanized Antibodies

In embodiments, the anti-BCMA antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

v. Human Antibodies

In embodiments, the anti-BCMA antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

vi. Library-Derived Antibodies

In embodiments, the anti-BCMA antibody provided herein is derived from an antibody library. Antibodies may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N J, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N J, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

vii. Multispecific Antibodies

In embodiments, the anti-BCMA antibody provided herein is a multispecific antibody, e.g. a bispecific antibody. Multispecific antibodies are monoclonal antibodies that have binding specificities for at least two different sites. In embodiments, one of the binding specificities is for BCMA and the other is for any other antigen. In embodiments, bispecific antibodies may bind to two different epitopes of BCMA. Bispecific antibodies may also be used to localize cytotoxic agents to cells which express BCMA. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); crosslinking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to BCMA as well as another, different antigen.

viii. Antibody Variants

In embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

a) Substitution, Insertion, and Deletion Variants

In embodiments, the anti-BCMA antibody provided herein has one or more amino acid substitutions. Sites of interest for substitutional mutagenesis include the HVRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1
Exemplary Amino acid substitutions.

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu:
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

b) Glycosylation Variants

In embodiments, an anti-BCMA antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In embodiments, modifications of the oligosaccharide in an antibody may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586: WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

c) Fe Region Variants

In embodiments, one or more amino acid modifications may be introduced into the Fc region of an anti-BCMA antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In embodiments, an antibody variant that possesses some but not all effector functions is contemplated, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656(1998). C1q binding assays may also be carried out to confirm that the antibody is unable to bind C1q and hence lacks CDC activity. See, e.g., C1q and C₃c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants. ix. Antibody Derivatives

In embodiments, an anti-BCMA antibody provided herein may be further modified to contain additional non-proteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

x. Recombinant Methods and Compositions

Antibodies may be produced using recombinant methods and compositions, e.g., as described in U.S. Pat. No. 4,816,567. One skilled in the art will be familiar with suitable host cells for antibody expression. Exemplary host cells include eukaryotic cells, e.g. a Chinese Hamster Ovary (CHO) cell or lymphoid cell (e.g., Y0, NS0, Sp20 cell).

For recombinant production of an anti-BCMA antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N J, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003); Dhara, V. G. et al., BioDrugs 32: 571-584 (2018); Kunert, R. and Reinhart, D. Applied microbiology and biotechnology, 100(8): 3451-3461 (2016).

xi. Assays

Anti-BCMA antibodies described herein may be identified, screened for, or characterized for their physical/chemical properties and/or biological activities by various assays known in the art.

In embodiment, an antibody is tested for its antigen binding activity, e.g., by known methods such as ELISA, BIACore®, FACS, or Western blot.

In another embodiment, competition assays may be used to identify an antibody that competes with any of the antibodies described herein for binding to BCMA. In embodiments, such a competing antibody binds to the same epitope (e.g., a linear or a conformational epitope) that is bound by an antibody described herein. Detailed exemplary methods for mapping an epitope to which an antibody binds are provided in Morris (1996) “Epitope Mapping Protocols,” in Methods in Molecular Biology vol. 66 (Humana Press, Totowa, NJ).

In an exemplary competition assay, immobilized BCMA is incubated in a solution comprising a first labeled antibody that binds to BCMA and a second unlabeled antibody that is being tested for its ability to compete with the first antibody for binding to BCMA. The second antibody may be present in a hybridoma supernatant. As a control, immobilized BCMA is incubated in a solution comprising the first labeled antibody but not the second unlabeled antibody. After incubation under conditions permissive for binding of the first antibody to BCMA, excess unbound antibody is removed, and the amount of label associated with immobilized BCMA is measured. If the amount of label associated with immobilized BCMA is substantially reduced in the test sample relative to the control sample, then that indicates that the second antibody is competing with the first antibody for binding to BCMA. In embodiments, immobilized BCMA is present on the surface of a cell or in a membrane preparation obtained from a cell expressing BCMA on its surface. See Harlow and Lane (1988) Antibodies: A Laboratory Manual ch. 14 (Cold Spring Harbor Laboratory, Cold Spring Harbor, NY).

Methods of Preparing Antibody-Drug Conjugates

An ADC of formula (I) may be prepared by several routes employing organic chemistry reactions, conditions, and reagents known to those skilled in the art, including: (1) reaction of a nucleophilic group of an antibody with a bivalent linker reagent (L¹) to form Ab-L¹ via a covalent bond, followed by reaction with a drug moiety D or drug-linker molecule D-L² and (2) reaction of a nucleophilic group of a drug moiety D with a bivalent linker reagent (L² and/or L¹) to form D-L² or D-L²-L¹ via a covalent bond, followed by reaction with a nucleophilic group of an antibody or a reduced antibody. Several such methods are described by Agarwal et al., (2015), Bioconjugate Chem., 26: 176-192.

In embodiments, an antibody may be reduced with a reducing agent such as dithiothreitol (DTT) or tricarbonylethylphosphine (TCEP), under partial or total reducing conditions, to generate reactive cysteine thiol groups. The inter-chain cysteine residues can then be alkylated for example using maleimide. Alternatively, the inter-chain cysteine residues can undergo bridging alkylation for example using bis sulfone linkers or propargyldibromomaleimide followed by Cu-click ligation. In embodiments, the antibody can be conjugated through lysine amino acid. Such conjugation can be a one-step conjugation or a two-step conjugation. In embodiments, the one-step conjugation entails conjugation of the ε-amino group of lysine residue to the drug-linker molecule (D-L²-L¹ or D-L¹) containing an amine-reactive group via amide bonds. In embodiments the amine-reactive group is an activated ester. In embodiments, the antibody can be conjugated via a two-step conjugation. The two-step conjugation entails a first step, where a bi-functional reagent containing both an amine and a thiol reactive functional groups is reacted with the lysine ε-amino group(s). In the second step, the drug-linker molecule (D-L²-L¹ or D-L¹) is conjugated to the thiol reactive group of the bifunctional reagent. Several examples are provided by Jain et al., (2015), Pharm. Res., 32:3526-3540. In embodiments, the first step may involve the functionalization of the antibody with azide followed by a click chemistry reaction with an alkyne modified linker or drug-linker molecule (D-L²-L¹ or D-L¹). In embodiments, the first step may involve the functionalization of the antibody with an alkyne followed by a click chemistry reaction with an azide modified linker or drug-linker molecule (D-L²-L¹ or D-L¹). In embodiments, the first step may involve the functionalization of the antibody with an aldehyde followed by a click chemistry reaction with a alkoxyamine or hydrazine modified linker or drug-linker molecule (D-L²-L¹ or D-L¹). In embodiments, the first step may involve the functionalization of the antibody with a tetrazine followed by a click chemistry reaction with a trans-cyclooctene or cyclopropene modified linker or drug-linker molecule (D-L²-L¹ or D-L¹). In embodiments, the first step may involve the functionalization of the antibody with a trans-cyclooctene or cyclopropene followed by a click chemistry reaction with a tetrazine modified linker or drug-linker molecule (D-L²-L¹ or D-L¹). Some examples are described by Pickens et al., (2018), Bioconjug. Chem., 29:686-701; Li et al., (2018), MAbs, 10:712-719; and Chio et al., (2020), Methods Mol. Biol., 2078:83-97.

In an aspect, an ADC of formula (I) can be prepared by reacting a monoclonal antibody (Ab) with a molecule of formula (P-I):

B-L²-D
or a pharmaceutically acceptable salt thereof, wherein:
B is a reactive moiety capable of forming a bond with the monoclonal antibody;
L² is a bond, —C(O)—, —NH—, Amino Acid Unit, —(CH₂CH₂O)ₙ—, —(CH₂)ₙ—, -(4-aminobenzyloxycarbonyl)-,

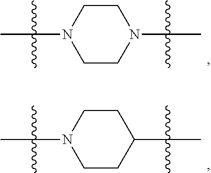 [SMILES: *N1CCN(*)CC1 ; *C1CCN(*)CC1]

—(C(O)CH₂CH₂NH)— or combinations thereof, where n is an integer from 1 to 24;
D is a drug moiety.

In an aspect, an ADC of formula (I) can be prepared by reacting an anti-BCMA, anti-ROR1, anti-CD25, or anti-Claudin 18 antibody (Ab) with a molecule of formula (P-I):

B-L²-D
or a pharmaceutically acceptable salt thereof, wherein:
B is a reactive moiety capable of forming a bond with the anti-BCMA, anti-ROR1, anti-CD25, or anti-Claudin 18 antibody;
L² is a bond, —C(O)—, —NH—, Amino Acid Unit, —(CH₂CH₂O)ₙ—, —(CH₂)ₙ—, -(4-aminobenzyloxycarbonyl)-,

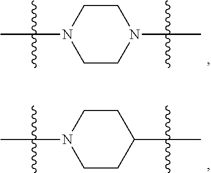 [SMILES: *N1CCN(*)CC1 ; *C1CCN(*)CC1]

—(C(O)CH₂CH₂NH)— or combinations thereof, where n is an integer from 1 to 24; D is a drug moiety.

In embodiments, the monoclonal antibody is modified with an aldehyde, azide, alkyne, tetrazine, hydrazine, alkoxyamine, trans-cyclooctene or cyclopropene. In embodiments, the monoclonal antibody is modified with an aldehyde. In embodiments, the monoclonal antibody is modified with an azide. In embodiments, the monoclonal antibody is modified with a tetrazine. In embodiments, the monoclonal antibody is modified with a alkoxyamine. In embodiments, the monoclonal antibody is modified with a hydrazine. In embodiments, the monoclonal antibody is modified with a trans-cyclooctene. In embodiments, the monoclonal antibody is modified with a cyclopropene.

In embodiments, Ab is an anti-BCMA, anti-ROR1, anti-CD25, or anti-Claudin 18 antibody. In embodiments, Ab is an anti-BCMA antibody. In embodiments, Ab is an anti-ROR1 antibody. In embodiments, Ab is an anti-CD25 antibody. In embodiments, Ab is an anti-Claudin 18 antibody. In embodiments, B is a reactive moiety capable of forming a bond with an anti-BCMA antibody. In embodiments, Ab is a modified anti-BCMA antibody.

In embodiments, Ab is modified with an aldehyde, azide, alkyne, tetrazine, hydrazine, alkoxyamine, trans-cyclooctene or cyclopropene. In embodiments, Ab is modified with an aldehyde. In embodiments, Ab is modified with an azide. In embodiments, Ab is modified with a tetrazine. In embodiments, Ab is modified with a alkoxyamine. In embodiments, Ab is modified with a hydrazine. In embodiments, Ab is modified with a trans-cyclooctene. In embodiments, Ab is modified with a cyclopropene. In embodiments, a modified Ab is a modified anti-BCMA antibody.

In embodiments, n is an integer from 1 to 24. In embodiments, n is 1. In embodiments, n is 2. In embodiments, n is 3. In embodiments, n is 4. In embodiments, n is 5. In embodiments, n is 6. In embodiments, n is 7. In embodiments, n is 8. In embodiments, n is 9. In embodiments, n is 10. In embodiments, n is 11. In embodiments, n is 12. In embodiments, n is 13. In embodiments, n is 14. In embodiments, n is 15. In embodiments, n is 16. In embodiments, n is 17. In embodiments, n is 18. In embodiments, n is 19. In embodiments, n is 20. In embodiments, n is 21. In embodiments, n is 22. In embodiments, n is 23. In embodiments, n is 24.

In embodiments, B is a reactive moiety capable of forming a bond with one or two thiol or amine groups of the anti-BCMA antibody, or with the modified anti-BCMA antibody. In embodiments, the anti-BCMA antibody is modified with an azide, aldehyde, alkyne, tetrazine, hydrazine, alkoxyamine, trans-cyclooctene or cyclopropene.

In embodiments, B is an alkyne, azide, aldehyde, tetrazine, hydrazine, alkoxyamine, trans-cyclooctene, cyclopropene, activated ester, haloacetyl, cycloalkyne, maleimide, or bis-sulfone. In embodiments, B is dibromomaleimide. In embodiments, B is cyclooctyne. In embodiments, the activated ester may be for example pentafluorophenyl ester, tetrafluorophenyl ester, trifluorophenyl ester, difluorophenyl ester, monofluorophenyl or ester, N-hydroxysuccinimide ester.

In embodiments, B is

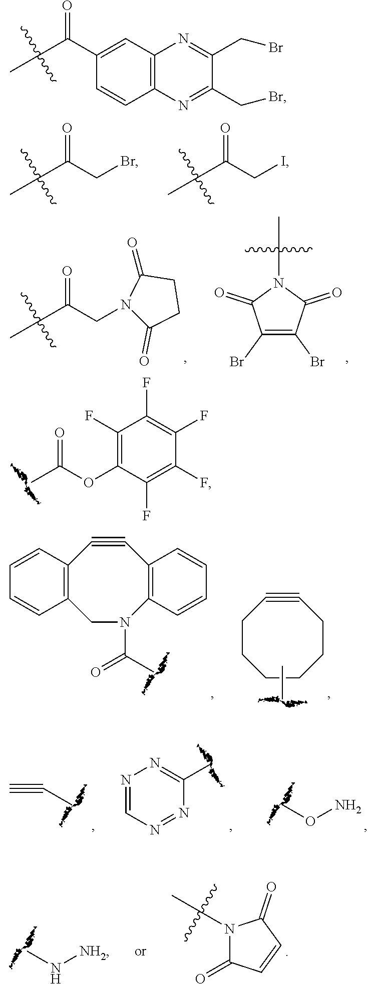

In embodiments, B is

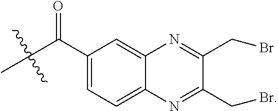 [SMILES: *C(=O)c1ccc2nc(CBr)c(CBr)nc2c1]

In embodiments, B is

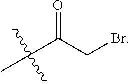 [SMILES: *C(=O)CBr]

In embodiments, B is

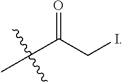 [SMILES: *C(=O)CI]

In embodiments, B is

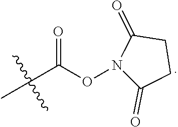 [SMILES: *C(=O)ON1C(=O)CCC1=O]

In embodiments, B is

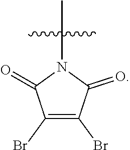 [SMILES: *N1C(=O)C(Br)=C(Br)C1=O]

In embodiments, B is

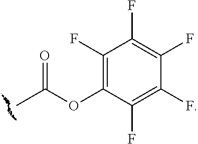

In embodiments, B is

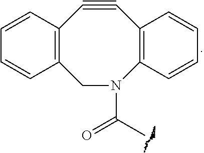 [SMILES: CC(=O)N1Cc2ccccc2C#Cc2ccccc21]

In embodiments, B is

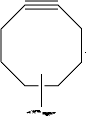

In embodiments, B is

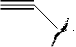

In embodiments, B is

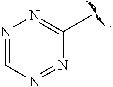

In embodiments, B is

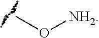

In embodiments, B is

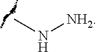

In embodiments, B is

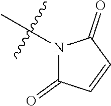 [SMILES: *N1C(=O)C=CC1=O]

In embodiments, B-L²- is

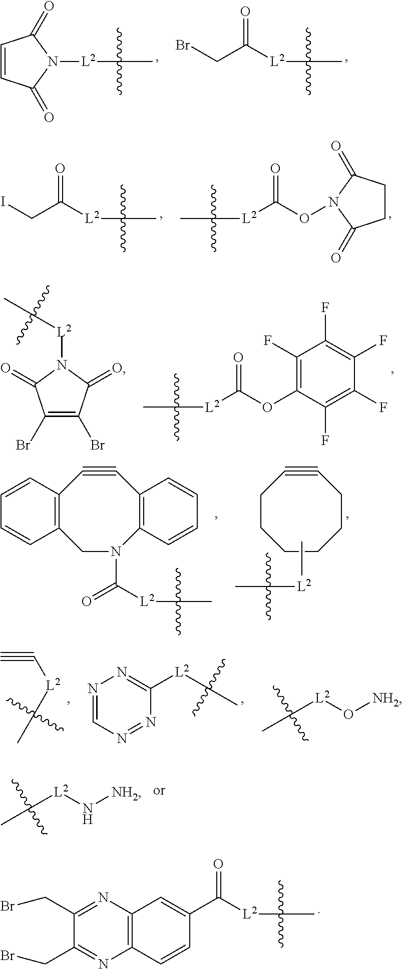

In embodiments, monoclonal antibodies, modified monoclonal antibodies, or anti-BCMA unmodified or modified antibodies (Ab) undergo conjugation reactions with the following reactive B moieties:

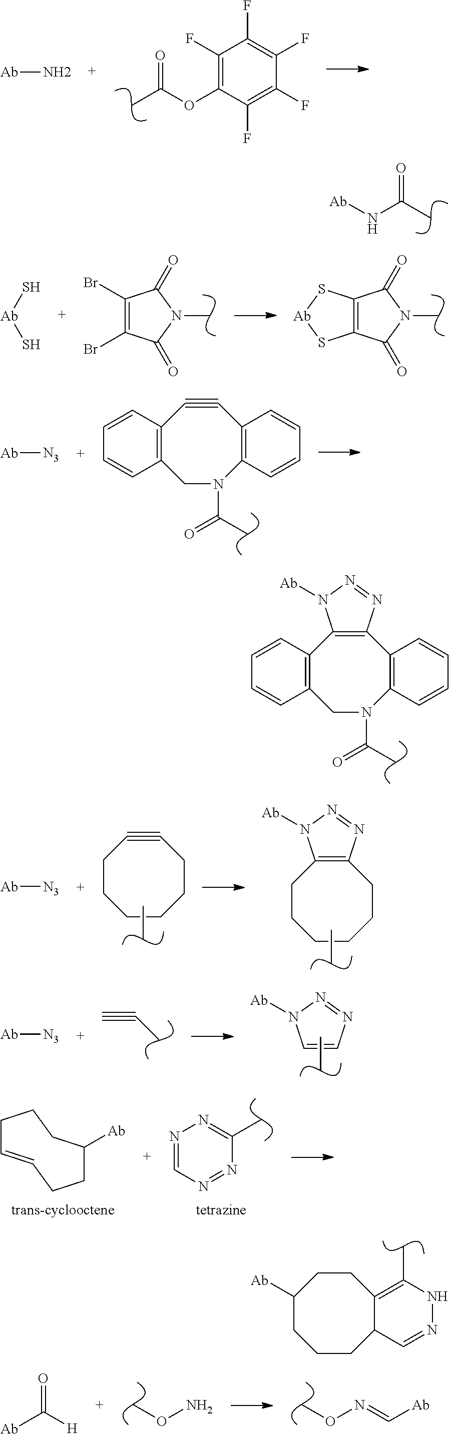

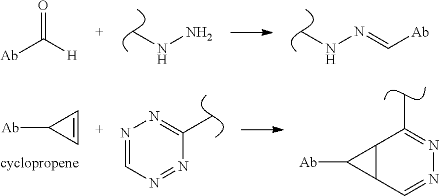

In embodiments, D is:

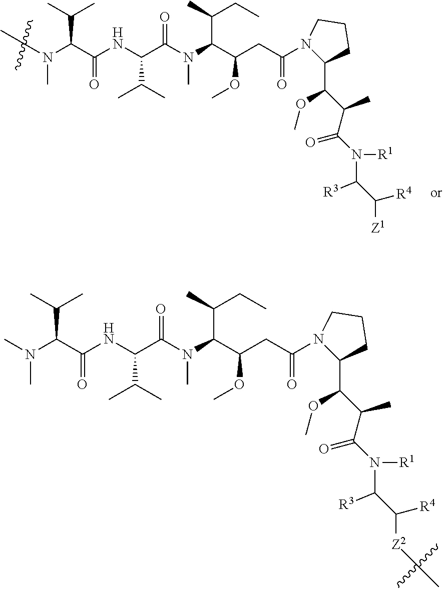

R¹ is H or —C₁-C₈ alkyl;
R³ is H, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OR^{3A}, —NR^{3A}R^{3B}, —(CH₂)ᵥOR⁶,

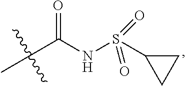 [SMILES: *C(=O)NS(=O)(=O)C1CC1]

substituted or unsubstituted alkyl, or substituted or unsubstituted heteroalkyl;
R⁴ is H, halogen, —OR^{4A}, —N^{4A}R^{4B}, substituted or unsubstituted alkyl, or substituted or unsubstituted heteroalkyl;
Z¹ is a substituted or unsubstituted aryl, substituted or unsubstituted heteroaryl, substituted or unsubstituted cycloalkyl, or substituted or unsubstituted heterocycloalkyl;
Z² is a substituted or unsubstituted arylene, substituted or unsubstituted heteroarylene, substituted or unsubstituted cycloalkylene, or substituted or unsubstituted heterocycloalkylene;
R⁶ is H, substituted or unsubstituted alkyl, substituted or unsubstituted cycloalkyl, substituted or unsubstituted heterocycloalkyl, substituted or unsubstituted aryl, substituted or unsubstituted heteroaryl, —CO(CH₂CH₂O)_{w}CH₂CH₂Y, —CONH(CH₂CH₂O)_{w}CH₂CH₂Y,

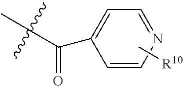

a Charged Group, or a saccharide derivative, wherein
v is an integer from 1 to 24; w is an integer from 1 to 24; Y is —NH₂, —OH, —COOH, or —OCH₃;
R¹⁰ is —OH, —OCH₃ or —COOH; and
each R^{3A}, R^{3B}, R^{4A}, and R^{4B} is independently H or substituted or unsubstituted alkyl.

In embodiments, L² is a cleavable or a non-cleavable linker as described in U.S. Pat. Nos. 9,884,127, 9,981,046, 9,801,951, 10,117,944, 10,590,165, and 10,590,165, and US Patent publications Nos. US 2017/0340750, and US 2018/0360985, all of which are incorporated herein in their entireties.

In embodiments, L² is a bond, —C(O)—, —NH—, -Val-, -Phe-, -Lys-, -(4-aminobenzyloxycarbonyl)-, -Gly-, -Ser-, -Thr-, -Ala-, -β-Ala-, -citrulline- (Cit),

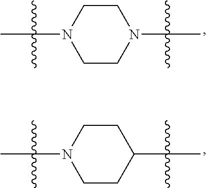 [SMILES: *N1CCN(*)CC1 ; *C1CCN(*)CC1]

—(CH₂)ₙ—, —(CH₂CH₂O)ₙ—, or combinations thereof.

In embodiments, L² is a bond, —C(O)—, —NH—, -Val-, -Phe-, -Lys-, -(4-aminobenzyloxycarbonyl)-,

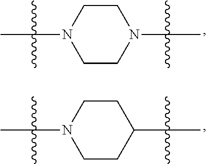

—(CH₂)ₙ—, —(CH₂CH₂O)ₙ—, or combinations thereof.

In embodiments, L² is a bond, —C(O)—, —NH—, -Gly-, -Ser-, -Thr-, -Ala-, -β-Ala-, -Cit-,

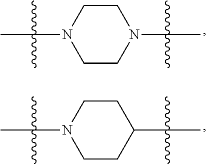

—(CH₂)ₙ—, —(CH₂CH₂O)ₙ—, or combinations thereof.

In embodiments, L² is

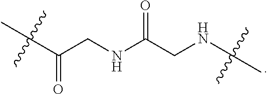 [SMILES: *NCC(=O)NCC(*)=O]

In embodiments, L² is

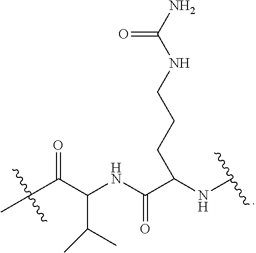 [SMILES: *NC(CCCNC(N)=O)C(=O)NC(C(*)=O)C(C)C]

In embodiments, L² is

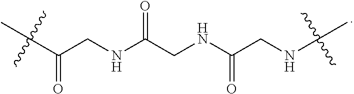 [SMILES: *NCC(=O)NCC(=O)NCC(*)=O]

In embodiments, L² is

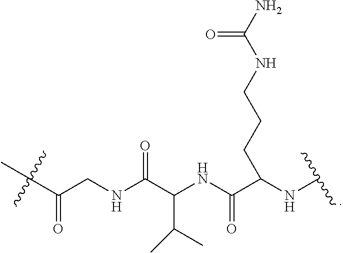 [SMILES: *NC(CCCNC(N)=O)C(=O)NC(C(=O)NCC(*)=O)C(C)C]

In embodiments, L² is

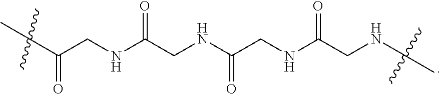 [SMILES: *NCC(=O)NCC(=O)NCC(=O)NCC(*)=O]

In embodiments, L² is

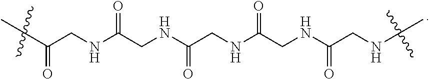 [SMILES: *NCC(=O)NCC(=O)NCC(=O)NCC(=O)NCC(*)=O]

In embodiments, L is —C(O)—(CH₂)₅—. In embodiments, L² is

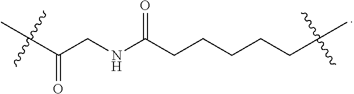 [SMILES: *CCCCCC(=O)NCC(*)=O]

In embodiments, L² is

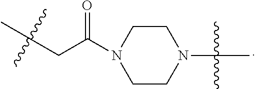 [SMILES: *CC(=O)N1CCN(*)CC1]

In embodiments, L² is

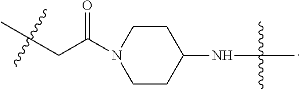 [SMILES: *CC(=O)N1CCC(N*)CC1]

In embodiments, L² is

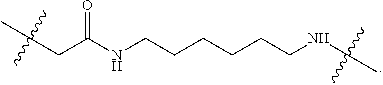 [SMILES: *CC(=O)NCCCCCCN*]

In embodiments, L² is

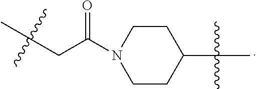 [SMILES: *CC(=O)N1CCC(*)CC1]

In embodiments, L² is a bond. In embodiments, L² is —C(O)—. In embodiments, L² is —NH—. In embodiments, L is -Val-. In embodiments, L² is -Phe-. In embodiments, L² is -Lys-. In embodiments, L² is -(4-aminobenzyloxycarbonyl)-. In embodiments, L² is —(CH₂)ₙ—. In embodiments, L² is —(CH₂CH₂O)ₙ—. In embodiments, L² is -Gly-. In embodiments, L² is -Ser-. In embodiments, L² is -Thr-. In embodiments, L² is -Ala-. In embodiments, L² is -β-Ala-. In embodiments, L² is -Cit-.

In embodiments, R¹ is H. In embodiments, R¹ is —C₁-C₈ alkyl.

In embodiments, R¹ is methyl, ethyl, propyl, isopropyl, butyl, isobutyl, tert-butyl, pentyl, or hexyl. In embodiments, R¹ is methyl. In embodiments, R¹ is ethyl. In embodiments, R¹ is propyl. In embodiments, R¹ is isopropyl. In embodiments, R¹ is butyl. In embodiments, R¹ is isobutyl. In embodiments, R¹ is tert-butyl. In embodiments, R¹ is pentyl. In embodiments, R¹ is hexyl.

In embodiments, R³ is H, halogen, -CCl₃—CBr₃, —CF₃, —CI₃, —CHCl₂, —CHBr₂, —CHF₂, —CHI₂, —CH₂CI, —CH₂Br, —CH₂F, —CH₂I, —CN, —OR^{3A}, —NR^{3A}R^{3B}, —(CH₂)ᵥOR⁶,

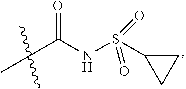 [SMILES: *C(=O)NS(=O)(=O)C1CC1]

substituted or unsubstituted alkyl (e.g., C₁-C₈ alkyl, C₁-C₆ alkyl, or C₁-C₄ alkyl), or substituted or unsubstituted heteroalkyl (e.g., 2 to 8 membered heteroalkyl, 2 to 6 membered heteroalkyl, or 2 to 4 membered heteroalkyl).

In embodiments, R³ is H, —OR^{3A}, —(CH₂) OR⁶,

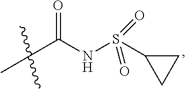 [SMILES: *C(=O)NS(=O)(=O)C1CC1]

substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl), or substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl).

In embodiments, R³ is a substituted (e.g., substituted with at least one substituent group, size-limited substituent group, or lower substituent group) alkyl (e.g., C₁-C₈ alkyl, C₁-C₆ alkyl, or C₁-C₄ alkyl). In embodiments, R³ is an unsubstituted alkyl (e.g., C₁-C₈ alkyl, C₁-C₆ alkyl, or C₁-C₄ alkyl). In embodiments, R³ is a substituted (e.g., substituted with at least one substituent group, size-limited substituent group, or lower substituent group) heteroalkyl (e.g., 2 to 8 membered heteroalkyl, 2 to 6 membered heteroalkyl, or 2 to 4 membered heteroalkyl). In embodiments, R³ is an unsubstituted heteroalkyl (e.g., 2 to 8 membered heteroalkyl, 2 to 6 membered heteroalkyl, or 2 to 4 membered heteroalkyl).

In embodiments, R³ is methyl, ethyl, propyl, butyl, —CH₂OH, —CH₂CH₂OH, —CH₂N₃, —CH₂CH₂N₃, —CH₂OCH₃, —CH₂OCH₂CH₃, —CH₂CH₂OCH₃, —CH₂CH₂OCH₂CH₃,

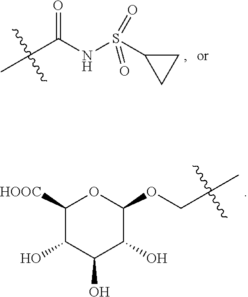 [SMILES: *C(=O)NS(=O)(=O)C1CC1 ; *CO[C@@H]1O[C@H](C(=O)O)[C@@H](O)[C@H](O)[C@H]1O]

In embodiments, R³ is methyl. In embodiments, R³ is ethyl. In embodiments, R³ is propyl. In embodiments, R³ is butyl. In embodiments, R³ is —CH₂OH. In embodiments, R³ is —CH₂ CH₂OH. In embodiments, R³ is —CH₂N₃. In embodiments, R³ is —CH₂CH₂N₃. In embodiments, R³ is —CH₂OCH₃. In embodiments, R³ is —CH₂OCH₂CH₃. In embodiments, R³ is —CH₂CH₂OCH₃. In embodiments, R³ is —CH₂CH₂OCH₂CH₃. In embodiments, R³ is —OH. In embodiments, R³ is H. In embodiments, R³ is

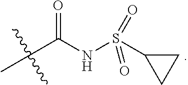 [SMILES: *C(=O)NS(=O)(=O)C1CC1]

In embodiments, R³ is

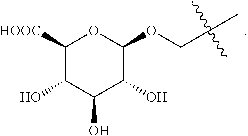 [SMILES: *CO[C@@H]1O[C@H](C(=O)O)[C@@H](O)[C@H](O)[C@H]1O]

In embodiments, R³ is methyl, —CH₂OH, —CH₂N₃,

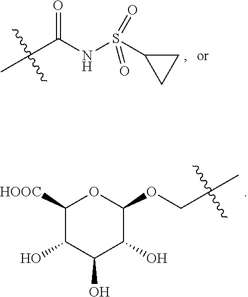 [SMILES: *C(=O)NS(=O)(=O)C1CC1 ; *CO[C@@H]1O[C@H](C(=O)O)[C@@H](O)[C@H](O)[C@H]1O]

In embodiments, R⁴ is H, halogen, —OR^{4A}, —NR^{4A}R^{4B}, substituted or unsubstituted alkyl (e.g., C₁-C₈ alkyl, C₁-C₆ alkyl, or C₁-C₄ alkyl), or substituted or unsubstituted heteroalkyl (e.g., 2 to 8 membered heteroalkyl, 2 to 6 membered heteroalkyl, or 2 to 4 membered heteroalkyl).

In embodiments, R⁴ is H, —OR^{4A}, substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl), or substituted (e.g., substituted with at least one substituent group, size-limited substituent group, or lower substituent group) or unsubstituted heteroalkyl (e.g., 2 to 8 membered heteroalkyl, 2 to 6 membered heteroalkyl, or 2 to 4 membered heteroalkyl).

In embodiments, R⁴ is a substituted (e.g., substituted with at least one substituent group, size-limited substituent group, or lower substituent group) alkyl (e.g., C₁-C₈ alkyl, C₁-C₆ alkyl, or C₁-C₄ alkyl). In embodiments, R⁴ is an unsubstituted alkyl (e.g., C₁-C₈ alkyl, C₁-C₆ alkyl, or C₁-C₄ alkyl). In embodiments, R⁴ is a substituted (e.g., substituted with at least one substituent group, size-limited substituent group, or lower substituent group) heteroalkyl (e.g., 2 to 8 membered heteroalkyl, 2 to 6 membered heteroalkyl, or 2 to 4 membered heteroalkyl). In embodiments, R⁴ is an unsubstituted heteroalkyl (e.g., 2 to 8 membered heteroalkyl, 2 to 6 membered heteroalkyl, or 2 to 4 membered heteroalkyl).

In embodiments, R⁴ is H, —OH, methyl, ethyl, propyl or butyl. In embodiments, R⁴ is methyl. In embodiments, R⁴ is ethyl. In embodiments, R⁴ is propyl. In embodiments, R⁴ is butyl. In embodiments, R⁴ is H. In embodiments, R⁴ is —OH.

In embodiments, R⁴ is H or —OH.

In embodiments, Z¹ is a substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl). In embodiments, Z¹ is a substituted (e.g. with a substituent group, a size-limited substituent group or a lower substituent group) cycloalkyl (e.g., C₃-C₈ cycloalkyl, C₃-C₆ cycloalkyl, or C₅-C₆ cycloalkyl). In embodiments, Z¹ is an unsubstituted cycloalkyl (e.g., C₃-C₈ cycloalkyl, C₃-C₆ cycloalkyl, or C₅-C₆ cycloalkyl). In embodiments, Z¹ is a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl). In embodiments, Z¹ is a substituted (e.g. with a substituent group, a size-limited substituent group or a lower substituent group) heterocycloalkyl (e.g., 3 to 8 membered heterocycloalkyl, 3 to 6 membered heterocycloalkyl, or 5 to 6 membered heterocycloalkyl). In embodiments, Z¹ is an unsubstituted heterocycloalkyl (e.g., 3 to 8 membered heterocycloalkyl, 3 to 6 membered heterocycloalkyl, or 5 to 6 membered heterocycloalkyl). In embodiments, Z¹ is a substituted (e.g. with a substituent group, a size-limited substituent group or a lower substituent group) or unsubstituted aryl (e.g., C₆-C₁₀ aryl, C₁₀ aryl, or phenyl). In embodiments, Z¹ is a substituted (e.g. with a substituent group, a size-limited substituent group or a lower substituent group) aryl (e.g., C₆-C₁₀ aryl, C₁₀ aryl, or phenyl). In embodiments, Z¹ is an unsubstituted aryl (e.g., C₆-C₁₀ aryl, C₁₀ aryl, or phenyl). In embodiments, Z¹ is a substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, Z¹ is a substituted (e.g. with a substituent group, a size-limited substituent group or a lower substituent group) heteroaryl (e.g., 5 to 10 membered heteroaryl, 5 to 9 membered heteroaryl, or 5 to 6 membered heteroaryl). In embodiments, Z¹ is an unsubstituted heteroaryl (e.g., 5 to 10 membered heteroaryl, 5 to 9 membered heteroaryl, or 5 to 6 membered heteroaryl).

In embodiments, Z¹ is

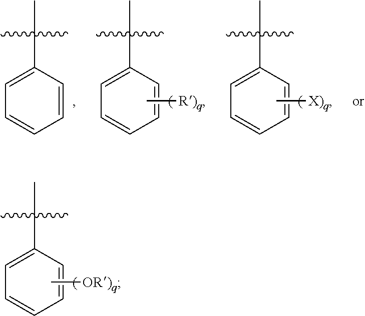

wherein each X is independently Cl, Br, I, or F; each R¹ is independently —CH₃, —CH₂CH₃ or —CH₂CH₂CH₃; and q is an integer from 1 to 5.

In embodiments, q is 1. In embodiments q is 2. In embodiments q is 3. In embodiments q is 4. In embodiments q is 5.

In embodiments, X is Cl. In embodiments, X is Br. In embodiments, X is I. In embodiments, X is F.

In embodiments, R¹ is —CH₃. In embodiments, R¹ is —CH₂CH₃. In embodiments, R¹ is —CH₂CH₂CH₃.

In embodiments, Z¹ is

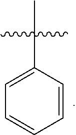 [SMILES: *c1ccccc1]

In embodiments, Z¹ is

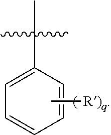

In embodiments, Z¹ is

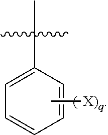

In embodiments, Z¹ is

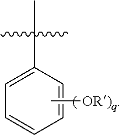

In embodiments, Z² is a substituted (e.g. with a substituent group, a size-limited substituent group or a lower substituent group) or unsubstituted cycloalkylene (e.g., C₃-C₈ cycloalkylene, C₃-C₆ cycloalkylene, or C₅-C₆ cycloalkylene). In embodiments, Z² is a substituted (e.g. with a substituent group, a size-limited substituent group or a lower substituent group) or unsubstituted heterocycloalkylene (e.g., 3 to 8 membered heterocycloalkylene, 3 to 6 membered heterocycloalkylene, or 5 to 6 membered heterocycloalkylene). In embodiments, Z² is a substituted (e.g. with a substituent group, a size-limited substituent group or a lower substituent group) or unsubstituted arylene (e.g., C₆-C₁₀ arylene, C₁₀ arylene, or phenylene). In embodiments, Z² is a substituted (e.g. with a substituent group, a size-limited substituent group or a lower substituent group) or unsubstituted heteroarylene (e.g., 5 to 10 membered heteroarylene, 5 to 9 membered heteroarylene, or 5 to 6 membered heteroarylene).

In embodiments, Z² is an unsubstituted arylene.

In embodiments, Z² is

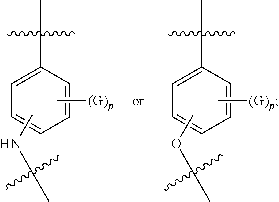

wherein each G is independently Cl, Br, I, F, —CH₃, —CH₂CH₃, —CH₂CH₂CH₃, —OCH₃, —OCH₂CH₃, —OH, or —NH₂; and p is an integer from 0-4.

In embodiments p is 0. In embodiments p is 1. In embodiments p is 2. In embodiments p is 3. In embodiments p is 4.

In embodiments, G is Cl. In embodiments, G is Br. In embodiments, G is I. In embodiments, G is F. In embodiments, G is —CH₃. In embodiments, G is —CH₂CH₃. In embodiments, G is —CH₂CH₂CH₃. In embodiments, G is —OCH₃. In embodiments, G is —OCH₂CH₃. In embodiments, G is —OH. In embodiments, G is —NH₂.

In embodiments, Z² is

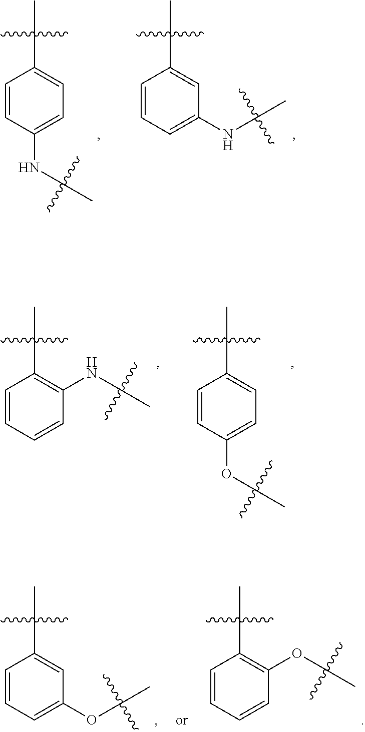 [SMILES: *Nc1ccc(*)cc1 ; *Nc1cccc(*)c1 ; *Nc1ccccc1* ; *Oc1ccc(*)cc1 ; *Oc1cccc(*)c1 ; *Oc1ccccc1*]

In embodiments, Z² is

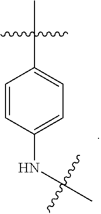 [SMILES: *Nc1ccc(*)cc1]

In embodiments, Z² is

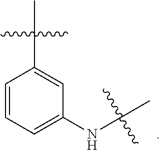 [SMILES: *Nc1cccc(*)c1]

In embodiments, Z² is

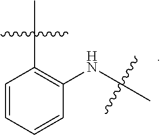 [SMILES: *Nc1ccccc1*]

In embodiments, Z² is

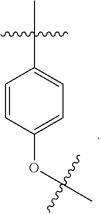 [SMILES: *Oc1ccc(*)cc1]

In embodiments, Z² is

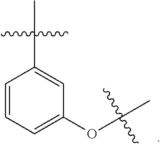 [SMILES: *Oc1cccc(*)c1]

In embodiments, Z² is

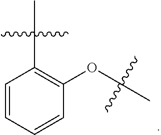 [SMILES: *Oc1ccccc1*]

In embodiments, R⁶ is H, substituted or unsubstituted alkyl, substituted or unsubstituted cycloalkyl, substituted or unsubstituted heterocycloalkyl, substituted or unsubstituted aryl, substituted or unsubstituted heteroaryl, —CO(CH₂CH₂O)_{w}CH₂CH₂Y, —CONH(CH₂CH₂O)_{w}CH₂CH₂Y,

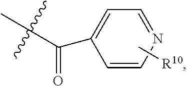

a Charged Group, or a saccharide derivative, w is an integer from 1 to 24; Y is —NH₂, —OH, —COOH, or —OCH₃; R¹⁰ is —OH, —OCH₃ or —COOH.

In embodiments, R⁶ is H or substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl), substituted (e.g., substituted with at least one substituent group, size-limited substituent group, or lower substituent group) or unsubstituted cycloalkyl (e.g., C₃-C₈ cycloalkyl, C₃-C₆ cycloalkyl, or C₅-C₆ cycloalkyl), substituted (e.g., substituted with at least one substituent group, size-limited substituent group, or lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g., substituted with at least one substituent group, size-limited substituent group, or lower substituent group) or unsubstituted aryl (e.g., C₆-C₁₀ aryl, C₁₀ aryl, or phenyl), substituted or unsubstituted heteroaryl (e.g., 5 to 10 membered heteroaryl, 5 to 9 membered heteroaryl, or 5 to 6 membered heteroaryl), or a saccharide derivative.

In embodiments, R⁶ is H, a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl). In embodiments, R⁶ is a substituted (e.g. with a substituent group, a size-limited substituent group or a lower substituent group) heterocycloalkyl (e.g., 3 to 8 membered heterocycloalkyl, 3 to 6 membered heterocycloalkyl, or 5 to 6 membered heterocycloalkyl). In embodiments, R⁶ is an unsubstituted heterocycloalkyl (e.g., 3 to 8 membered heterocycloalkyl, 3 to 6 membered heterocycloalkyl, or 5 to 6 membered heterocycloalkyl).

In embodiments, R⁶ is H or substituted (e.g. with a substituent group, a size-limited substituent group or a lower substituent group) heterocycloalkyl (e.g., 3 to 8 membered heterocycloalkyl, 3 to 6 membered heterocycloalkyl, or 5 to 6 membered heterocycloalkyl).

In embodiments, R⁶ is H or

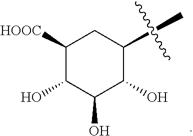

In embodiments, R⁶ is —CO(CH₂CH₂O)*CH₂CH₂Y or —CONH(CH₂CH₂O)_{w}CH₂CH₂Y, where w is an integer from 1 to 24 and Y is —NH₂, —OH, —COOH, or —OCH₃. In embodiments, R⁶ is —CO(CH₂CH₂O)_{w}CH₂CH₂NH₂. In embodiments, R⁶ is —CO(CH₂CH₂O)_{w}CH₂CH₂OH. In embodiments, R⁶ is —CO(CH₂CH₂O)_{w}CH₂CH₂COOH. In embodiments, R⁶ is —CO(CH₂CH₂O)_{w}CH₂CH₂OCH₃. In embodiments, R⁶ is —CONH(CH₂CH₂O)_{w}CH₂CH₂NH₂. In embodiments, R⁶ is —CONH(CH₂CH₂O)_{w}CH₂CH₂OH. In embodiments, R⁶ is —CONH(CH₂CH₂O)_{w}CH₂CH₂COOH. In embodiments, R⁶ is —CONH(CH₂CH₂O)_{w}CH₂CH₂OCH₃.

In embodiments, w is an integer from 1 to 24. In embodiments, w is 1. In embodiments, w is 2. In embodiments, w is 3. In embodiments, w is 4. In embodiments, w is 5. In embodiments, w is 6. In embodiments, w is 7. In embodiments, w is 8. In embodiments, w is 9. In embodiments, w is 10. In embodiments, w is 11. In embodiments, w is 12. In embodiments, w is 13. In embodiments, w is 14. In embodiments, w is 15. In embodiments, w is 16. In embodiments, w is 17. In embodiments, w is 18. In embodiments, w is 19. In embodiments, w is 20. In embodiments, w is 21. In embodiments, w is 22. In embodiments, w is 23. In embodiments, w is 24.

In embodiments, Y is —NH₂, —OH, —COOH, or —OCH₃ In embodiments, Y is —NH₂. In embodiments, Y is —OH. In embodiments, Y is —COOH. In embodiments, Y is —OCH₃.

In embodiments, R⁶ is

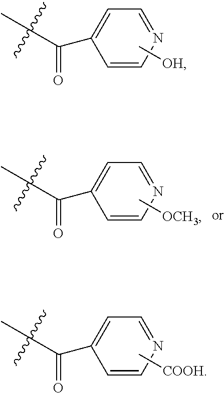

In embodiments, R⁶ is

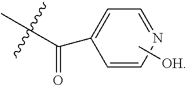

In embodiments, R⁶ is

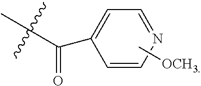

In embodiments, R⁶ is

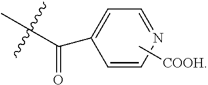

In embodiments, R⁶ is a saccharide derivative. In embodiments, R⁶ is

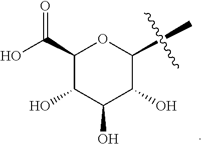

In embodiments, R⁶ is

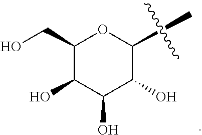

In embodiments, R⁶ is

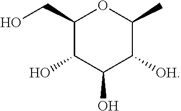

In embodiments, each R^{3A}, R^{3B}, R^{4A}, and R^{4B} is independently H or substituted or unsubstituted alkyl (e.g., C₁-C₈ alkyl, C₁-C₆ alkyl, or C₁-C₄ alkyl).

In embodiments, each R^{3A}, R^{3B}, R^{4A}, and R^{4B} is independently H or substituted (e.g., substituted with at least one substituent group, size-limited substituent group, or lower substituent group) or unsubstituted alkyl (e.g., C₁-C₈ alkyl, C₁-C₆ alkyl, or C₁-C₄ alkyl). In embodiments, each R^{3A}, R^{3B}, R^{4A}, and R^{4B} is independently H. In embodiments, each R^{3A}, R^{3B}, R^{3A}, and R^{4B} is independently substituted (e.g., substituted with at least one substituent group, size-limited substituent group, or lower substituent group) alkyl (e.g., C₁-C₈ alkyl, C₁-C₆ alkyl, or C₁-C₄ alkyl). In embodiments, each R^{3A}, R^{3B}, R^{4A}, and R^{4B} is independently unsubstituted alkyl (e.g., C₁-C₈ alkyl, C₁-C₆ alkyl, or C₁-C₄ alkyl).

In embodiments, each R^{3A}, R^{3B}, R^{4A}, and R^{4B} is independently H, methyl, ethyl, propyl, isopropyl, butyl, isobutyl, tert-butyl, or pentyl. In embodiments, each R^{3A}, R^{3B}, R^{4A}, and R^{4B} is independently H. In embodiments, each R^{3A}, R^{3B}, R^{4A}, and R^{4B} is independently methyl. In embodiments, each R^{3A}, R^{3B}, R^{4A}, and R^{4B} is independently ethyl. In embodiments, each R^{3A}, R^{3B}, R^{4A}, and R^{4B} is independently propyl. In embodiments, each R^{3A}, R^{3B}, R^{4A}, and R^{4B} is independently isopropyl. In embodiments, each R^{3A}, R^{3B}, R^{4A}, and R^{4B} is independently butyl. In embodiments, each R^{3A}, R^{3B}, R^{4A}, and R^{4B} is independently isobutyl. In embodiments, each R^{3A}, R^{3B}, R^{4A}, and R^{4B} is independently tert-butyl. In embodiments, each R^{3A}, R^{3B}, R^{4A}, and R^{4B} is independently pentyl.

In embodiments, D is:

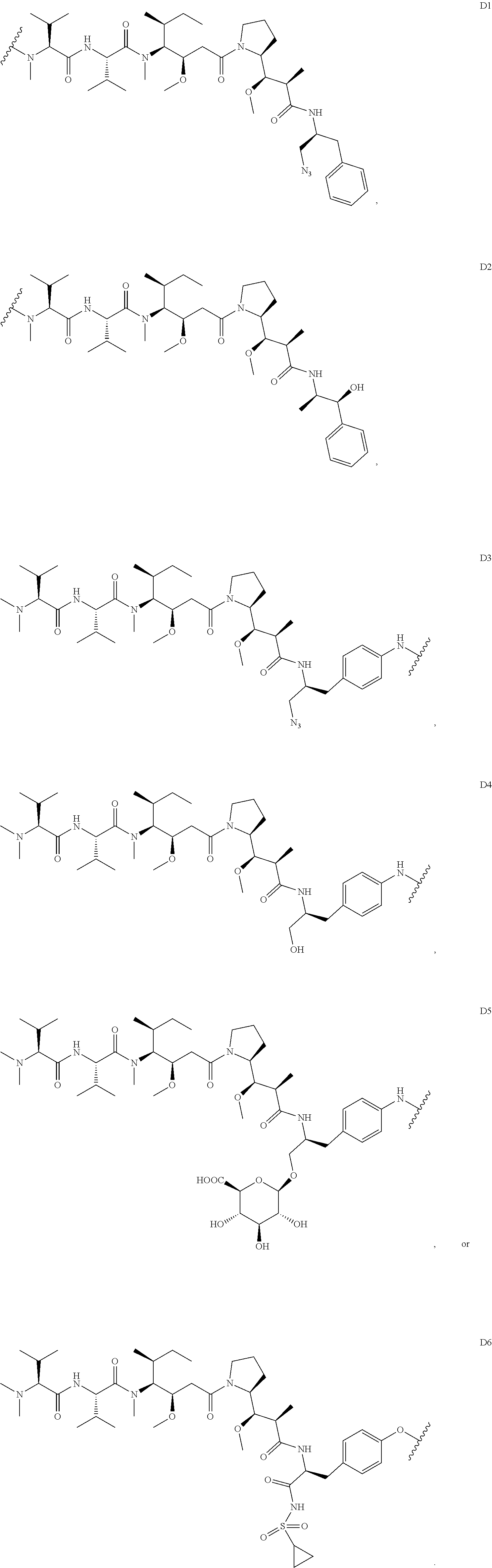

In embodiments, D is:

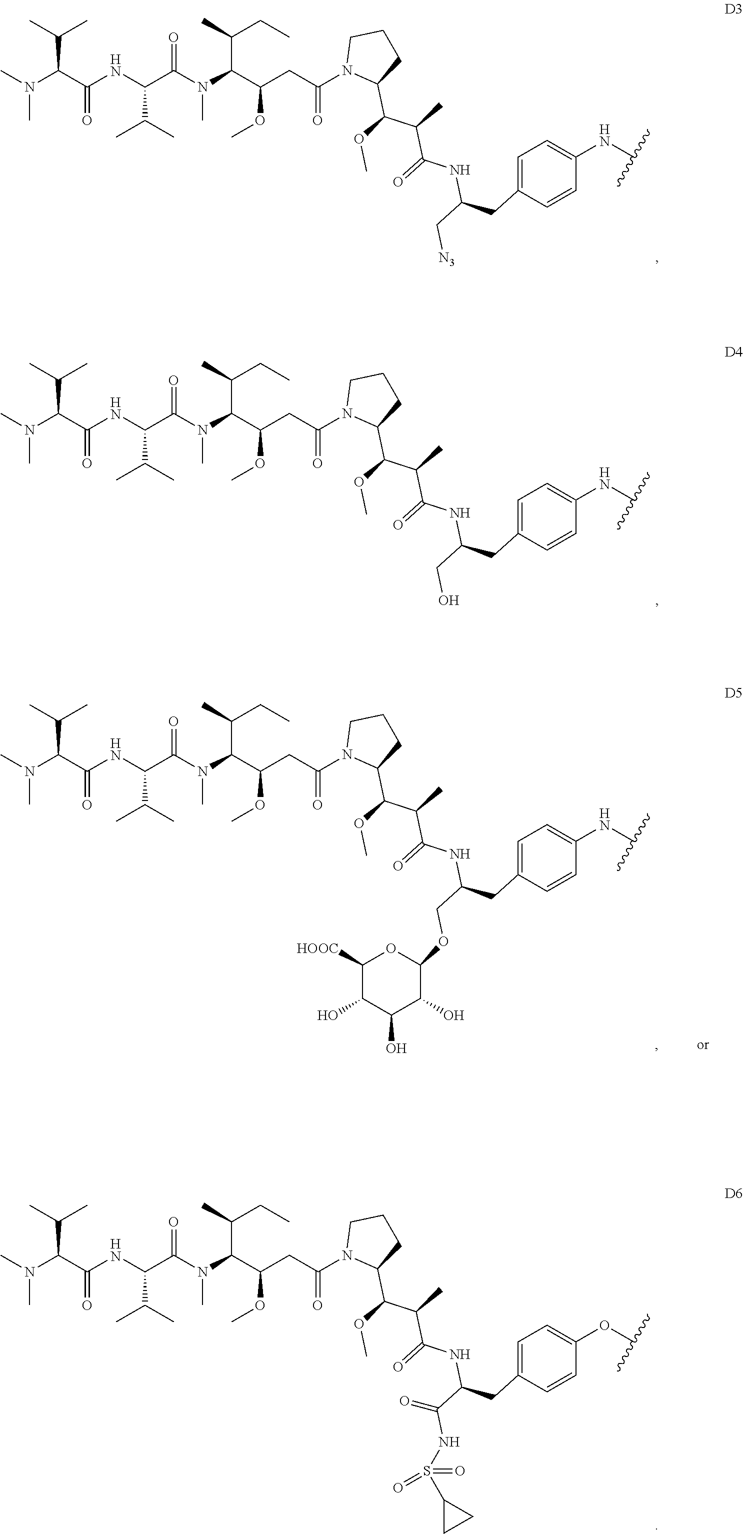

In embodiments, D is:

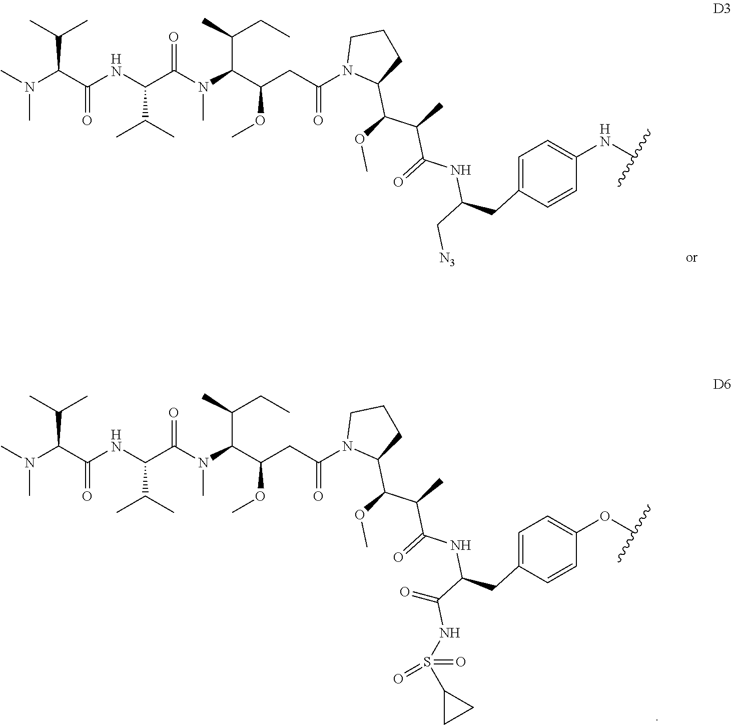 [SMILES: *Nc1ccc(C[C@@H](CN=[N+]=[N-])NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)cc1 ; *Oc1ccc(C[C@H](NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)C(=O)NS(=O)(=O)C2CC2)cc1]

In embodiments, D is:

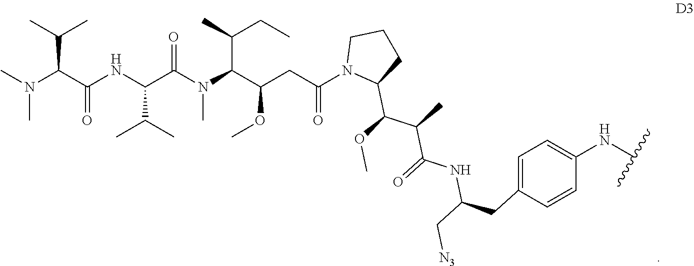 [SMILES: *Nc1ccc(C[C@@H](CN=[N+]=[N-])NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)cc1]

In embodiments, D is:

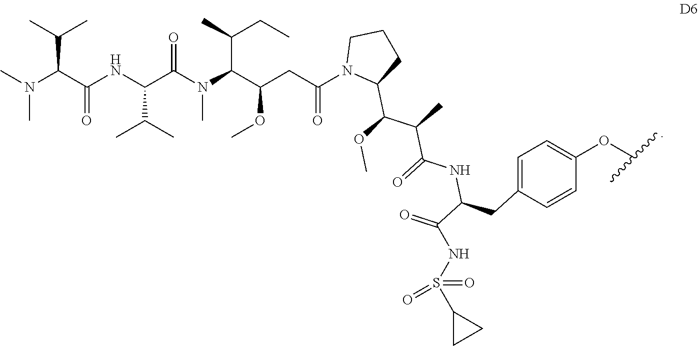 [SMILES: *Oc1ccc(C[C@H](NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)C(=O)NS(=O)(=O)C2CC2)cc1]

In embodiments, D is:

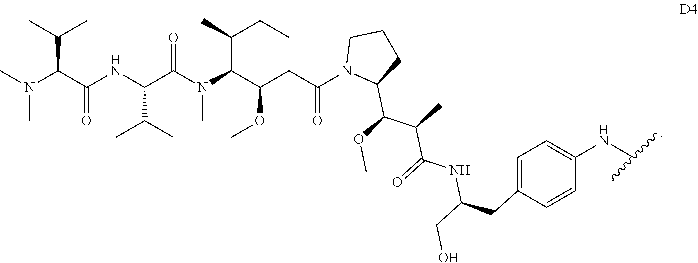 [SMILES: *Nc1ccc(C[C@@H](CO)NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)cc1]

In embodiments, D is:

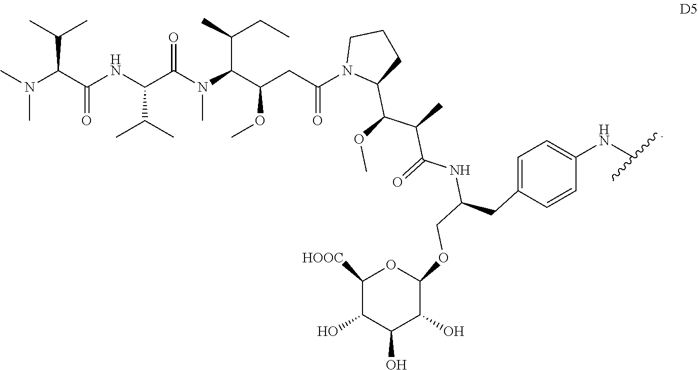 [SMILES: *Nc1ccc(C[C@@H](CO[C@@H]2O[C@H](C(=O)O)[C@@H](O)[C@H](O)[C@H]2O)NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)cc1]

In embodiments, the molecule of formula (P-I) is a molecule of formula:

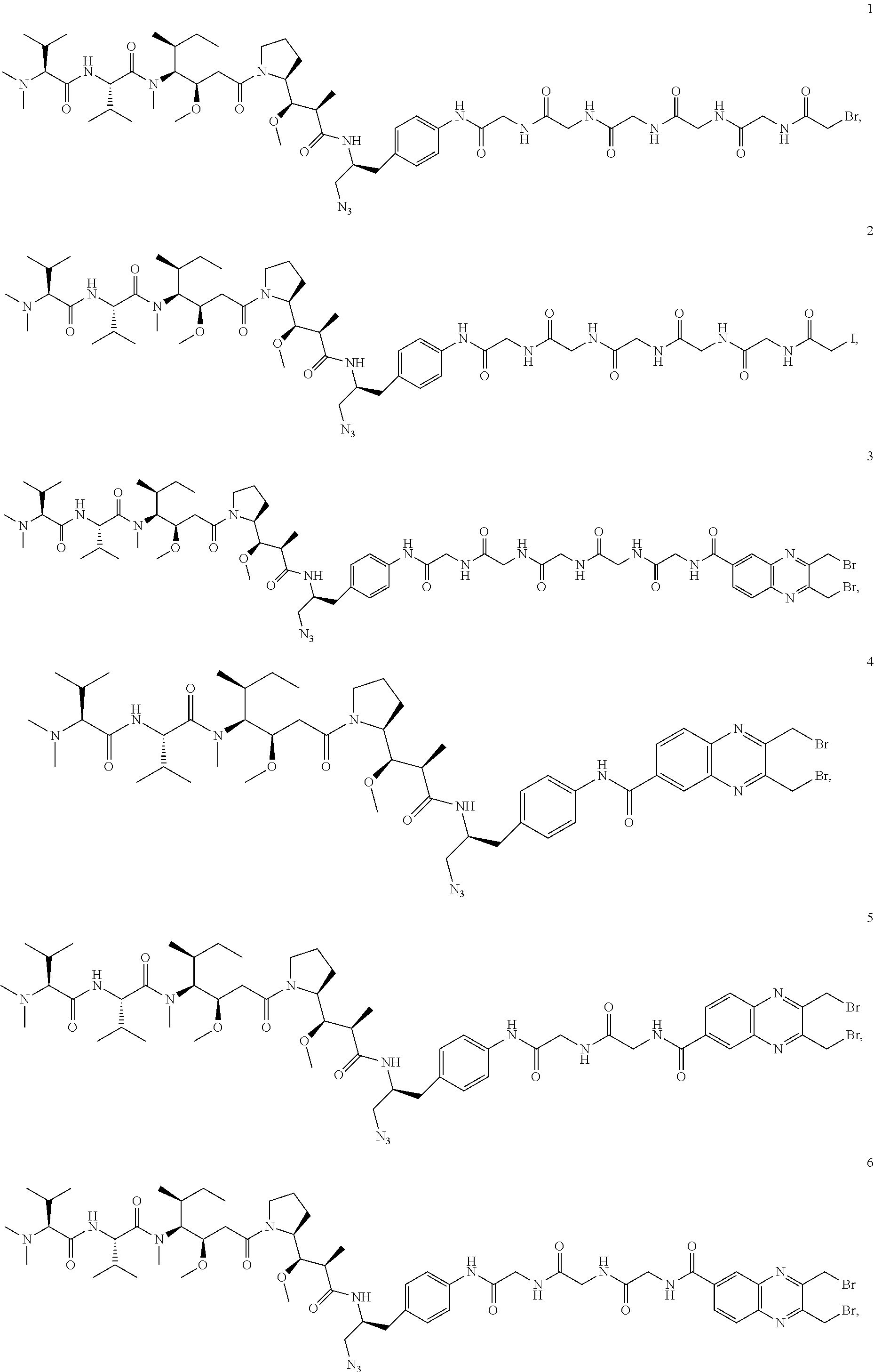

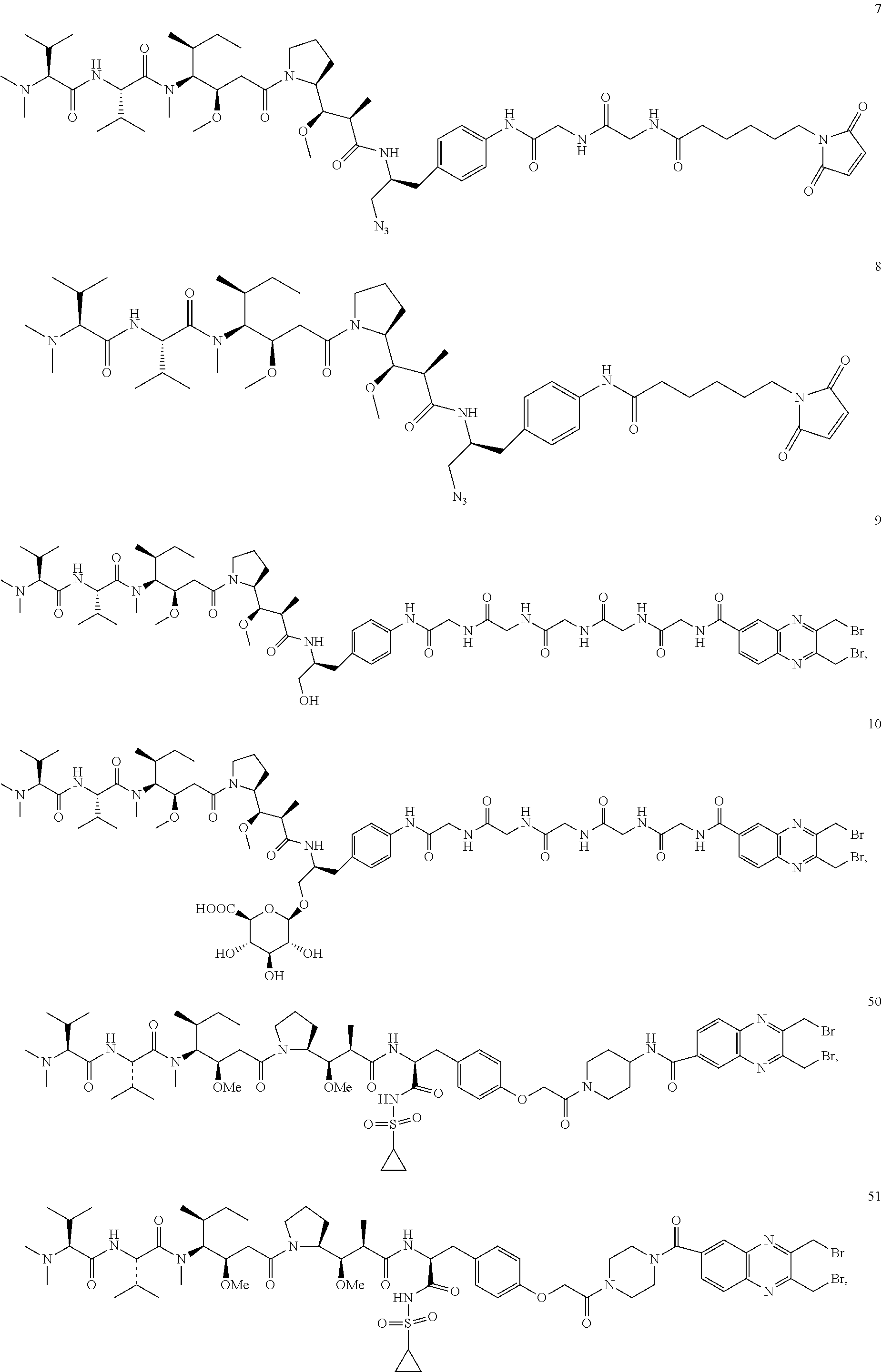

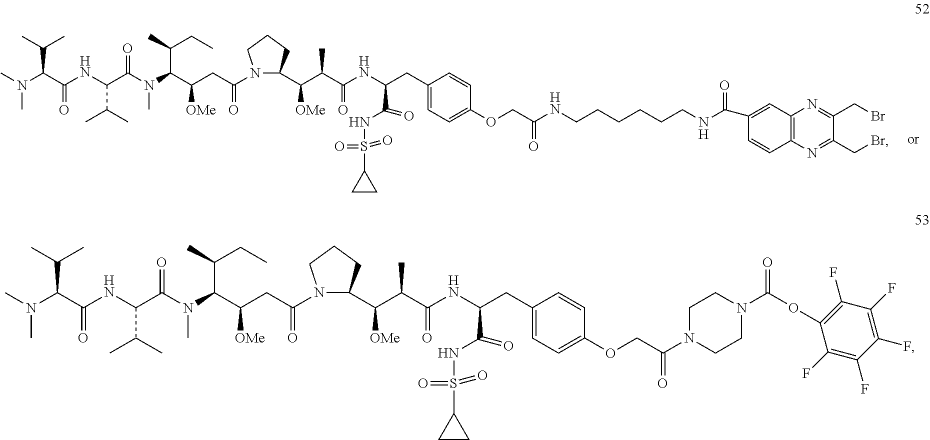 [SMILES: CC[C@H](C)[C@@H]([C@@H](CC(=O)N1CCC[C@H]1[C@H](OC)[C@@H](C)C(=O)N[C@@H](Cc1ccc(OCC(=O)NCCCCCCNC(=O)c2ccc3nc(CBr)c(CBr)nc3c2)cc1)C(=O)NS(=O)(=O)C1CC1)OC)N(C)C(=O)[C@@H](NC(=O)[C@H](C(C)C)N(C)C)C(C)C ; "or" ; CC[C@H](C)[C@@H]([C@@H](CC(=O)N1CCC[C@H]1[C@H](OC)[C@@H](C)C(=O)N[C@@H](Cc1ccc(OCC(=O)N2CCN(C(=O)Oc3c(F)c(F)c(F)c(F)c3F)CC2)cc1)C(=O)NS(=O)(=O)C1CC1)OC)N(C)C(=O)[C@@H](NC(=O)[C@H](C(C)C)N(C)C)C(C)C]

or a pharmaceutically acceptable salt thereof.

Pharmaceutical Compositions

In an aspect, provided herein is a pharmaceutical composition including an ADC as described herein, including embodiments, and a pharmaceutically acceptable carrier. In embodiments, the ADC as described herein is included in a therapeutically effective amount.

In embodiments, the pharmaceutical composition is formulated as a tablet, a powder, a capsule, a pill, a cachet, or a lozenge as described herein. The pharmaceutical composition may be formulated as a tablet, capsule, pill, cachet, or lozenge for oral administration. The pharmaceutical composition may be formulated for dissolution into a solution for administration by such techniques as, for example, intravenous administration. The pharmaceutical composition may be formulated for oral administration, suppository administration, topical administration, intravenous administration, intraperitoneal administration, intramuscular administration, intralesional administration, intrathecal administration, intranasal administration, subcutaneous administration, implantation, transdermal administration, or transmucosal administration as described herein.

The ADCs and pharmaceutical compositions thereof are particularly useful for parenteral administration, i.e., subcutaneously (s.c.), intrathecally, intraperitoneally, intramuscularly (i.m.) or intravenously (i.v.) In embodiment, the ADCs and pharmaceutical compositions thereof are administered intravenously or subcutaneously.

The compositions may contain pharmaceutically acceptable auxiliary substances as required to approximate physiological conditions such as pH adjusting and buffering agents, etc. The concentration of the antigen binding protein of the invention in such pharmaceutical formulation can vary widely, i.e., from less than about 0.5%, usually at or at least about 1% to as much as about 15 or 20% by weight and will be selected primarily based on fluid volumes, viscosities, etc., according to the particular mode of administration selected.

Actual methods for preparing parenterally administrable compositions are well known or will be apparent to those skilled in the art and are described in more detail in, for example, Remington's Pharmaceutical Science, 15^{th} ed., Mack Publishing Company, Easton, Pa. For the preparation of intravenously administrable antigen binding protein formulations of the invention see Lasmar U and Parkins D “The formulation of Biopharmaceutical products”, Pharma. Sci Tech. today, page 129-137, Vol. 3 (3 Apr. 2000), Wang, W “Instability, stabilisation and formulation of liquid protein pharmaceuticals”, Int. J. Pharm 185 (1999) 129-188; Stability of Protein Pharmaceuticals Part A and B ed Ahern T. J., Manning M. C., New York, N.Y.: Plenum Press (1992); Akers, M. J. “Excipient-Drug interactions in Parenteral Formulations”, J. Pharm Sci 91 (2002) 2283-2300; Imamura, K et al “Effects of types of sugar on stabilization of Protein in the dried state”, J Pharm Sci 92 (2003) 266-274; Izutsu, Kkojima, S. “Excipient crystallinity and its protein-structure-stabilizing effect during freeze-drying”, J Pharm Pharmacol, 54 (2002) 1033-1039; Johnson, R, “Mannitol-sucrose mixtures-versatile formulations for protein peroxidise19g19n”, J. Pharm Sci, 91 (2002) 914-922; and Ha, E Wang W, Wang Y. j. “Peroxide formation in polysorbate 80 and protein stability”, J. Pharm Sci, 91, 2252-2264, (2002) the entire contents of which are incorporated herein by reference and to which the reader is specifically referred.

In embodiments, the pharmaceutical composition may include optical isomers, diastereomers, enantiomers, isoforms, polymorphs, hydrates, solvates or products, or pharmaceutically acceptable salts of the compound described herein. The compound described herein (including pharmaceutically acceptable salts thereof) included in the pharmaceutical composition may be covalently attached to a carrier moiety, as described above. In embodiments, the compound described herein (including pharmaceutically acceptable salts thereof) included in the pharmaceutical composition is not covalently linked to a carrier moiety. A combination of covalently and not covalently linked compound described herein may be in a pharmaceutical composition herein.

Methods of Use

In an aspect, provided herein is a method of treating a disease in a subject in need thereof, said method including administering an effective amount of an antibody drug conjugate (ADC) comprising an IgG antibody, a conjugation linker moiety (L′) that binds to the thiol of cysteine residues or to the amine of lysine residues - of the IgG antibody, and to a drug moiety covalently bound to either L¹, or optionally another linker L². In embodiments, the IgG antibody binds to BCMA.

In one aspect, an ADC provided herein is used in a method of inhibiting proliferation of a BCMA-expressing cell, the method comprising exposing the cell to the ADC under conditions permissive for binding of the anti-BCMA antibody of the ADC on the surface of the cell, thereby inhibiting the proliferation of the cell. In embodiments, the method is an in vitro or an in vivo method In embodiments, the cell is a B cell.

Inhibition of cell proliferation in vitro may be assayed using the CellTiter-Glo™ Luminescent Cell Viability Assay, which is commercially available from Promega (Madison, WI). That assay determines the number of viable cells in culture based on quantitation of ATP present, which is an indication of metabolically active cells. See Crouch et al. (1993) J. Immunol. Meth. 160:81-88, U.S. Pat. No. 6,602,677. The assay may be conducted in 96- or 384-well format, making it amenable to automated high-throughput screening (HTS). See Cree et al. (1995) AntiCancer Drugs 6:398-404. The assay procedure involves adding a single reagent (CellTiter-Glo® Reagent) directly to cultured cells. This results in cell lysis and generation of a luminescent signal produced by a luciferase reaction. The luminescent signal is proportional to the amount of ATP present, which is directly proportional to the number of viable cells present in culture. Data can be recorded by luminometer or CCD camera imaging device. The luminescence output is expressed as relative light units (RLU).

In another aspect, an ADC for use as a medicament is provided. In further aspects, an ADC for use in a method of treatment is provided. In another aspect, provided herein is a method of treating a disease in a subject in need thereof, said method including administering an effective amount of a pharmaceutical composition of the ADC as described herein.

In embodiments, the disease is cancer. In embodiments, the cancer is associated with overexpression of BCMA. In embodiments, provided herein is an ADC for use in a method of treating an individual having a BCMA-expressing cancer, the method comprising administering to the individual an effective amount of the ADC. In one such embodiment, the method further comprises administering to the individual an effective amount of at least one additional therapeutic agent.

In a further aspect, the present disclosure provides for the use of an ADC in the manufacture or preparation of a medicament. In embodiment, the medicament is for treatment of BCMA-expressing cancer. In a further embodiment, the medicament is for use in a method of treating BCMA-expressing cancer, the method comprising administering to an individual having BCMA-expressing cancer an effective amount of the medicament. In one such embodiment, the method further comprises administering to the individual an effective amount of at least one additional therapeutic agent.

In embodiments, the methods provided herein are for treating cancer in a mammal. In embodiments, the methods provided herein are for treating cancer in a human.

In embodiments, the cancer is a B-cell mediated or plasma cell mediated disease or antibody mediated disease or disorder selected from the group consisting of Multiple Myeloma (MM), chronic lymphocytic leukemia (CLL), Non-secretory multiple myeloma. Smoldering multiple myeloma, Monoclonal gammopathy of undetermined significance (MGUS), Solitary plasmacytoma (Bone, Extramedullary), Lymphoplasmacytic lymphoma (LPL), Waldenstrom's Macroglobulinemia, Plasma cell leukemia-Primary Amyloidosis (AL), Heavy chain disease, Systemic lupus erythematosus (SLE), POEMS syndrome/osteosclerotic myeloma, Type I and II cryoglobulinemia, Light chain deposition disease, Goodpasture's syndrome, Idiopathic thrombocytopenic purpura (ITP), Acute glomerulonephritis, Pemphigus and Pemphigoid disorders, and Epidenmolysis bullosa acquisita; or any Non-Hodgkin's Lymphoma B-cell leukemia or Hodgkin's lymphoma (HL) with BCMA expression.

In embodiments, the cancer is selected from the group consisting of Multiple Myeloma (MM), Chronic Lymphocytic Leukaemia (CLL), Solitary Plasmacytoma (Bone, Extramedullary), and Waldenstrom's Macroglobulinemia

In embodiments, the cancer is Multiple Myeloma (MM).

### List of Sequences

Human BCMA sequence
SEQ ID NO: 16
MLQMAGQCSQNEYFDSLLHACIPCQLRCSSNTPPLTCQR
YCNASVTNSVKGTNAILWTCLGLSLIISLAVFVLMFLLR
KINSEPLKDEFKNTGSGLLGMANIDLEKSRTGDEIILPR
GLEYTVEECTCEDCIKSKPKVDSDHCFPLPAMEEGATIL
VTTKTNDYCKSLPAALSATEIEKSISAR.

Table of Sequences:

TABLE 2

| Light chain variable: | Heavy chain variable: |
|---|---|
| BCA7-2C5 SEQ ID NO: 7 | BCA7-2C5 SEQ ID NO: 8 |
| QSVLTQPASVSGSPGQSVTI | EVQLVESGGGLVKPGGSLRL |
| SCTGTSSAHGGHYYVSWYQQ | SCAASGFTSSTAWMSWVRQA |
| HPGKAPKLMIYDVSNRPSGV | PGKGLEWVGRIKSKSDGGTT |
| SNRFSGSKSGNTASLTISGL | DYAAPVKGRFTISRDDSKNT |
| QAEDEADYYCGSYTSSGSYV | LFLQMNSLKTEDTAVYYCAK |
| FGTGTKLTVL | GGGTYGYWGQGTTVTVSS |
| BCA7-2E1 SEQ ID NO: 15 | BCA7-2E1 SEQ ID NO: 8 |
| QSALTQPASVSGSPGQSVTI | EVQLVESGGGLVKPGGSLRL |
| SCTGTSSDGGGHTYVSWYQQ | SCAASGFTSSTAWMSWVRQA |
| HPGKAPKLMIYDVSNRPSWV | PGKGLEWVGRIKSKSDGGTT |
| SNRFSGSKSGNTASLTISGL | DYAAPVKGRFTISRDDSKNT |
| QAEDEADYYCGSYTSSGSYV | LFLQMNSLKTEDTAVYYCAK |
| FGTGTKLTVL | GGGTYGYWGQGTTVTVSS |

TABLE 3
CDRs 1, 2 and 3:
BCA7-2C5:
2C5 (VL-CDR1) SEQ ID NO: 1 TGTSSAHGGHYYVS
2C5 (VL-CDR2) SEQ ID NO: 2 DVSNRPS
2C5 (VL-CDR3) SEQ ID NO: 3 GSYTSSGSYV
2C5 (VH-CDR1) SEQ ID NO: 4 TAWMS
2C5 (VH-CDR2) SEQ ID NO: 5 RIKSKSDGGTTDYAAPVKG
2C5 (VH-CDR3) SEQ ID NO: 6 AKGGGTYGY
BCA7-2E1:
2E1 (VL-CDR1) SEQ ID NO: 9 TGTSSDGGGHTYVS
2E1 (VL-CDR2) SEQ ID NO: 10 DVSNRPS
2E1 (VL-CDR3) SEQ ID NO: 11 GSYTSSGSYV
2E1 (VH-CDR1) SEQ ID NO: 12 TAWMS
2E1 (VH-CDR2) SEQ ID NO: 13 RIKSKSDGGTTDYAAPVKG
2E1 (VH-CDR3) SEQ ID NO: 14 AKGGGTYGY

### EXAMPLES

The following examples are meant to be illustrative and can be used to further understand embodiments of the present disclosure and should not be construed as limiting the scope of the present teachings in any way.

The chemical reactions described in the Examples can be readily adapted to prepare a number of other compounds of the present disclosure, and alternative methods for preparing the compounds of this disclosure are deemed to be within the scope of this disclosure. For example, the synthesis of non-exemplified compounds according to the present disclosure can be successfully performed by modifications apparent to those skilled in the art, e.g., by utilizing other suitable reagents known in the art other than those described, or by making routing modifications of reaction conditions, reagents, and starting materials. Alternatively, other reactions disclosed herein or known in the art will be recognized as having applicability for preparing other compounds of the present disclosure. Synthesis of compound 40 and related compounds was disclosed in U.S. Pat. Nos. 10,590,165 and 9,981,046, which are incorporated herein in their entireties.

### SYNTHETIC EXAMPLES

### Example S1: Synthesis of Compound 1

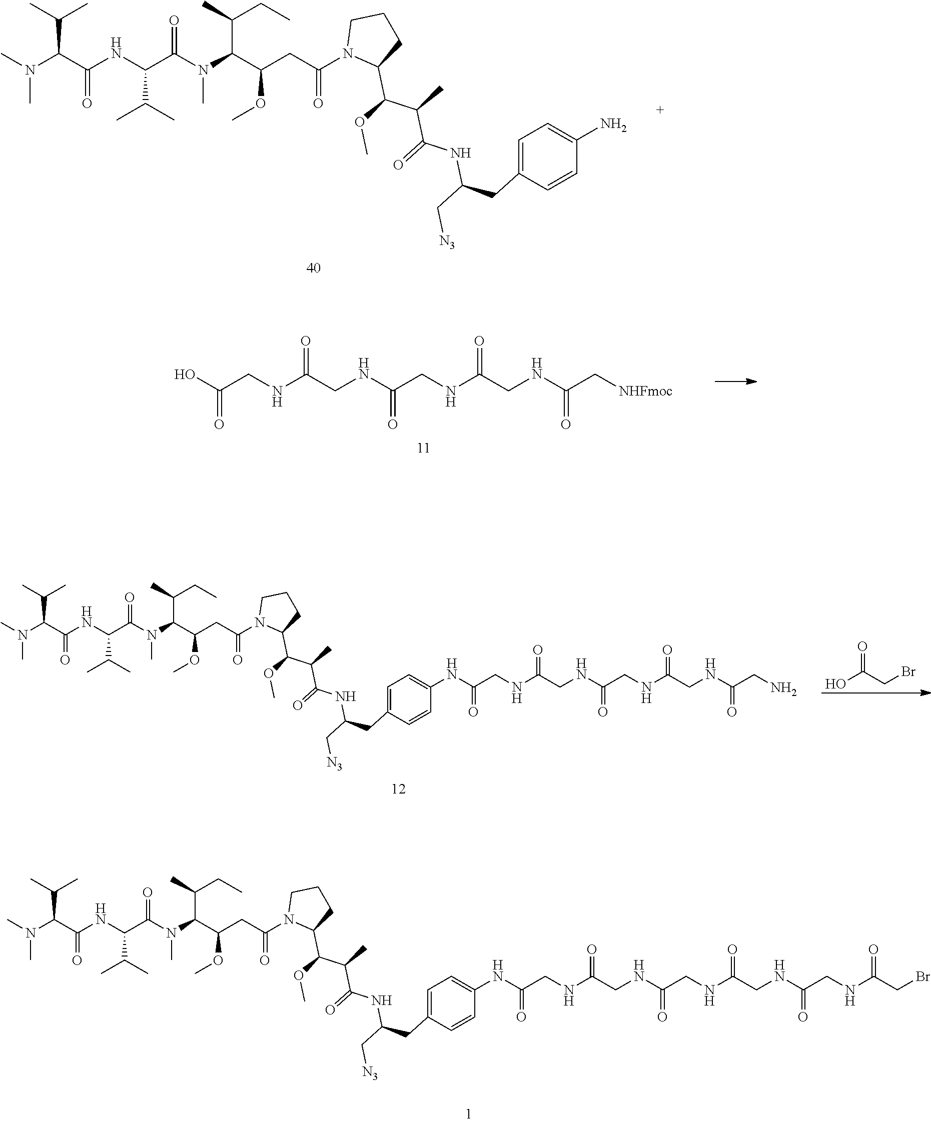

To compound 40 (TFA salt, 250 mg, 0.25 mmol) in 2 mL of DMF was added a solution of HATU (103 mg, 0.27 mmol), DIEA (188 μL, 1.08 mmol), and acid 11 (142 mg, 0.27 mmol) in 2 mL of DMF. The mixture was stirred for 30 min, then 160 μL of DBU was added and stirred for 10 min. The mixture was purified by HPLC to give compound 12 (214 mg). MS m/z 1057.6 (M+H).

To compound 12 (TFA salt, 6.2 mg, 4.8 μmol) in 0.5 mL of DMF was added a solution of bromoacetic acid (1.5 mg, 10.6 μmol), DIC (0.6 mg, 4.8 μmol), and DIEA (4 μL) in 0.5 mL of DCM. The mixture was stirred for 5 min, then purified by HPLC to give compound 1 (2.9 mg). MS m/z 1177.6 (M+H).

### Example S2: Synthesis of Compound 2

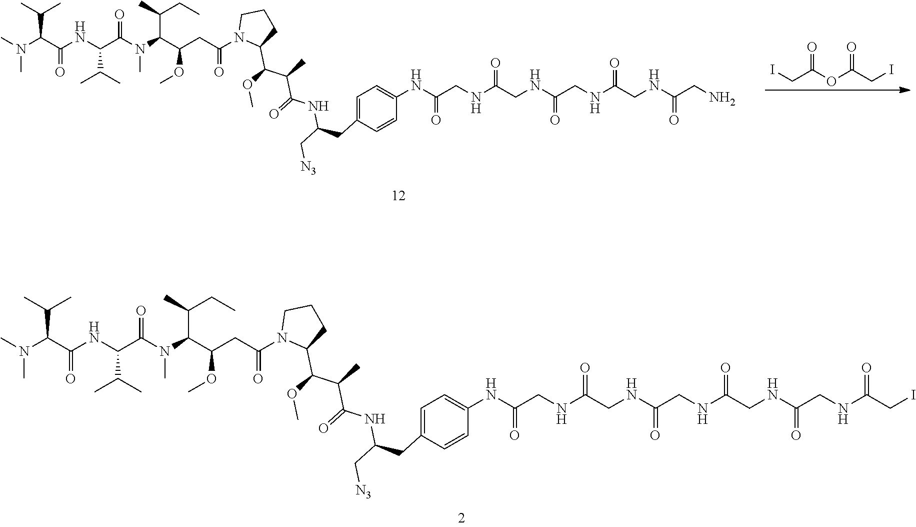 [SMILES: CC[C@H](C)[C@@H]([C@@H](CC(=O)N1CCC[C@H]1[C@H](OC)[C@@H](C)C(=O)N[C@H](CN=[N+]=[N-])Cc1ccc(NC(=O)CNC(=O)CNC(=O)CNC(=O)CNC(=O)CN)cc1)OC)N(C)C(=O)[C@@H](NC(=O)[C@H](C(C)C)N(C)C)C(C)C ; O=C(CI)OC(=O)CI ; CC[C@H](C)[C@@H]([C@@H](CC(=O)N1CCC[C@H]1[C@H](OC)[C@@H](C)C(=O)N[C@H](CN=[N+]=[N-])Cc1ccc(NC(=O)CNC(=O)CNC(=O)CNC(=O)CNC(=O)CNC(=O)CI)cc1)OC)N(C)C(=O)[C@@H](NC(=O)[C@H](C(C)C)N(C)C)C(C)C]

To compound 12 (TFA salt, 6.2 mg, 4.8 μmol) in 0.5 mL of DMF was added a solution of iodoacetic anhydride (2.0 mg, 5.6 μmol) and DIEA (4 μL) in 0.5 mL of DCM. The mixture was stirred for 5 min, then purified by HPLC to give compound 2 (5.6 mg). MS m/z 1224.7 (M+H).

### Example S3: Synthesis of Compound 3

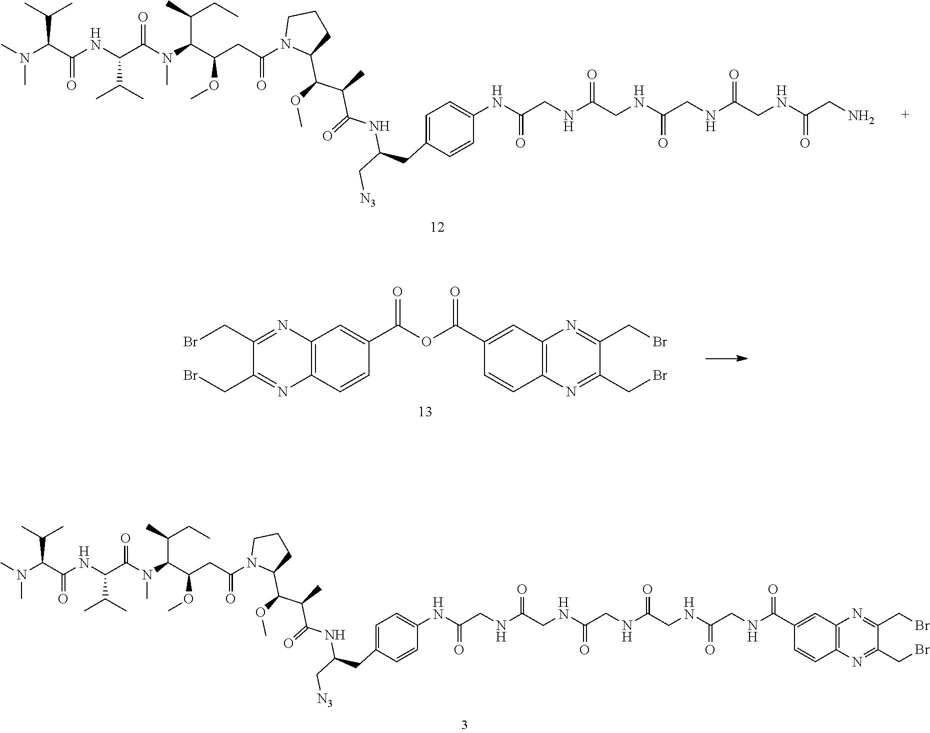 [SMILES: CC[C@H](C)[C@@H]([C@@H](CC(=O)N1CCC[C@H]1[C@H](OC)[C@@H](C)C(=O)N[C@H](CN=[N+]=[N-])Cc1ccc(NC(=O)CNC(=O)CNC(=O)CNC(=O)CNC(=O)CN)cc1)OC)N(C)C(=O)[C@@H](NC(=O)[C@H](C(C)C)N(C)C)C(C)C ; O=C(OC(=O)c1ccc2nc(CBr)c(CBr)nc2c1)c1ccc2nc(CBr)c(CBr)nc2c1 ; CC[C@H](C)[C@@H]([C@@H](CC(=O)N1CCC[C@H]1[C@H](OC)[C@@H](C)C(=O)N[C@H](CN=[N+]=[N-])Cc1ccc(NC(=O)CNC(=O)CNC(=O)CNC(=O)CNC(=O)CNC(=O)c2ccc3nc(CBr)c(CBr)nc3c2)cc1)OC)N(C)C(=O)[C@@H](NC(=O)[C@H](C(C)C)N(C)C)C(C)C]

To compound 12 (TFA salt, 10 mg, 7.8 μmol) in 0.5 mL of DMF was added a solution of anhydride 13 (16.5 mg, 23.5 μmol) and DIEA (5.4 μL, 31 μmol) in 0.5 mL of DMF. The mixture was stirred for 10 min, then purified by HPLC to give compound 3 (8.5 mg). MS m/z 1397.5 (M+H).

### Example S4: Synthesis of Compound 4

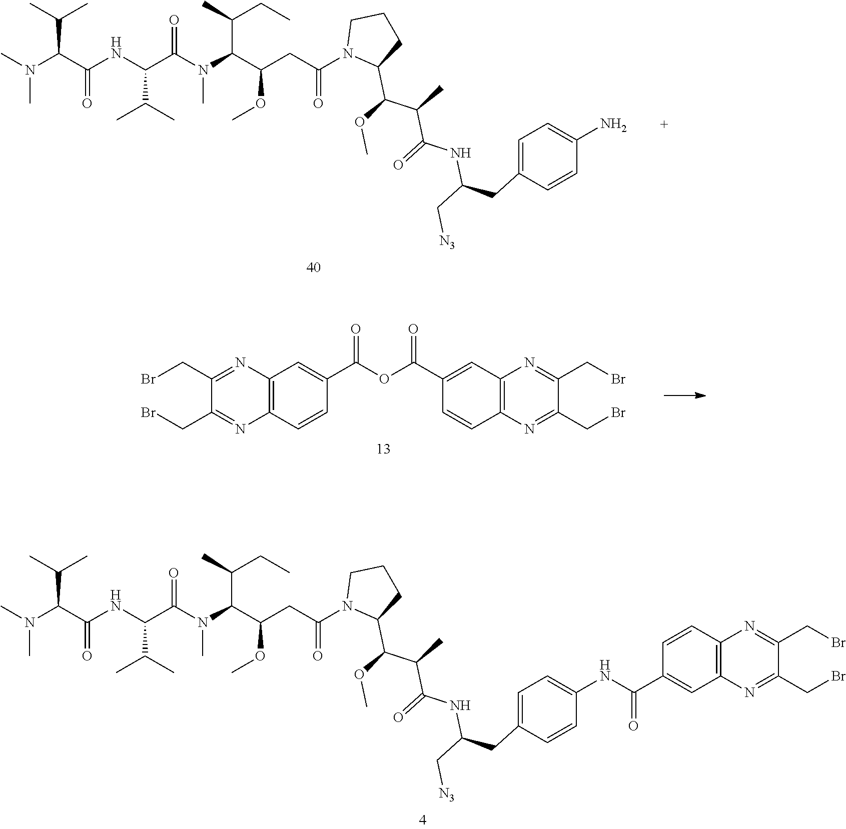 [SMILES: CC[C@H](C)[C@@H]([C@@H](CC(=O)N1CCC[C@H]1[C@H](OC)[C@@H](C)C(=O)N[C@H](CN=[N+]=[N-])Cc1ccc(N)cc1)OC)N(C)C(=O)[C@@H](NC(=O)[C@H](C(C)C)N(C)C)C(C)C ; O=C(OC(=O)c1ccc2nc(CBr)c(CBr)nc2c1)c1ccc2nc(CBr)c(CBr)nc2c1 ; CC[C@H](C)[C@@H]([C@@H](CC(=O)N1CCC[C@H]1[C@H](OC)[C@@H](C)C(=O)N[C@H](CN=[N+]=[N-])Cc1ccc(NC(=O)c2ccc3nc(CBr)c(CBr)nc3c2)cc1)OC)N(C)C(=O)[C@@H](NC(=O)[C@H](C(C)C)N(C)C)C(C)C]

To compound 40 (TFA salt, 14 mg, 16 μmol) in 1 mL of DMF was added a solution of anhydride 13 (14 mg, 20 μmol) and DIEA (11 μL, 63 μmol) in 1 mL of DMF. The mixture was stirred for 10 min, then purified by HPLC to give compound 4 (11.6 mg). MS m/z 1112.5 (M+H).

### Example S5: Synthesis of Compound 5

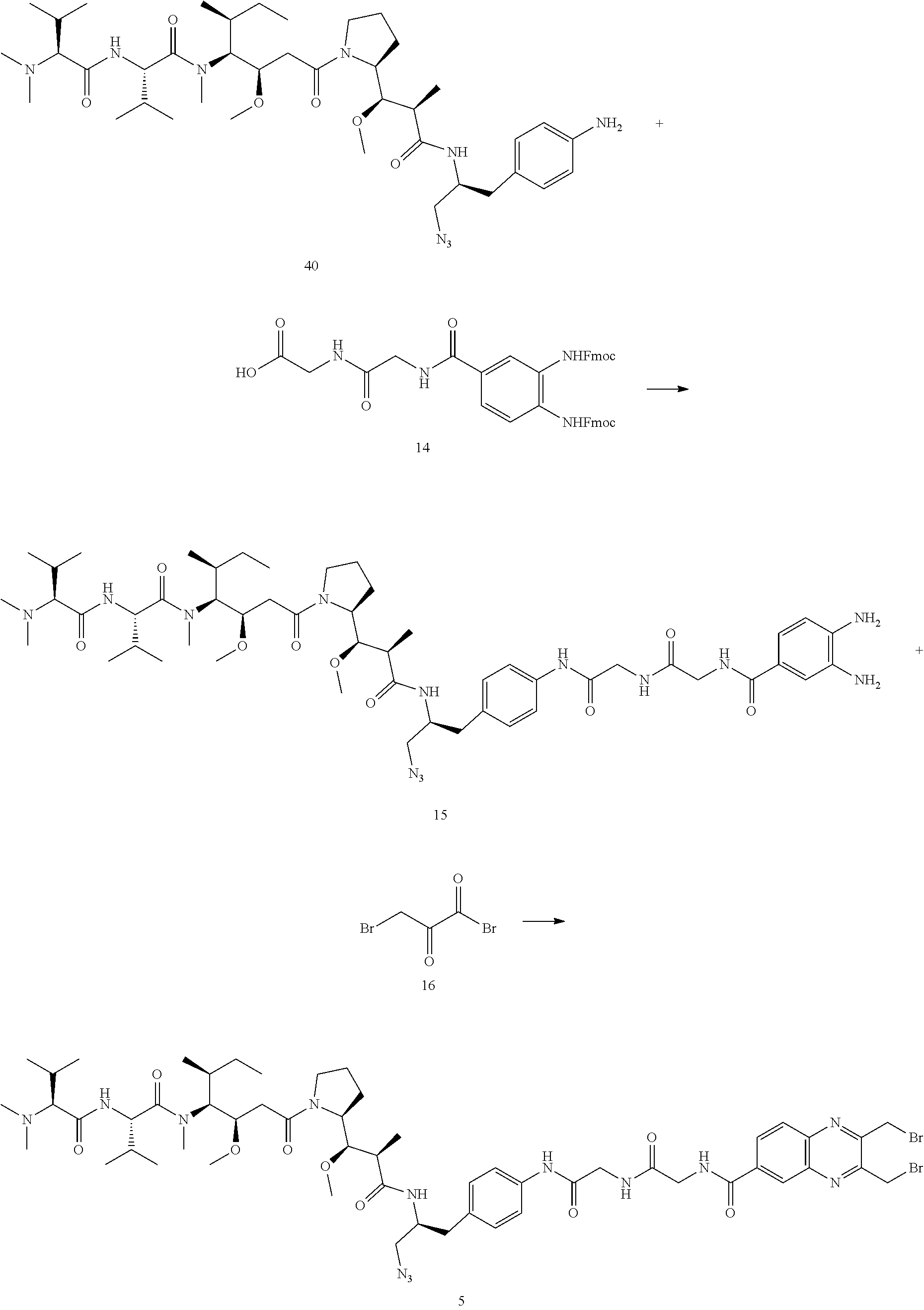

To compound 40 (TFA salt, 57 mg, 64.8 μmol) in 2 mL of DMF was added compound 14 (55 mg, 77.8 μmol), DIEA (34 μL, 0.2 mmol), and PyAOP (40 mg, 76.8 μmol). The mixture was stirred for 10 min, then 210 μL, of piperidine was added and stirred for 20 min. The mixture was purified by HPLC to give compound 15 (65 mg). MS m/z 1020.7 (M+H).

To compound 15 (TFA salt, 44 mg, 35 μmol) in 2 mL of acetonitrile and 1 mL of water was added bromide 16 (17 mg, 70 μmol). The mixture was stirred for 5 min, then purified by HPLC to give compound 5 (47.9 mg). MS m/z 1226.6 (M+H).

### Example S6: Synthesis of Compound 6

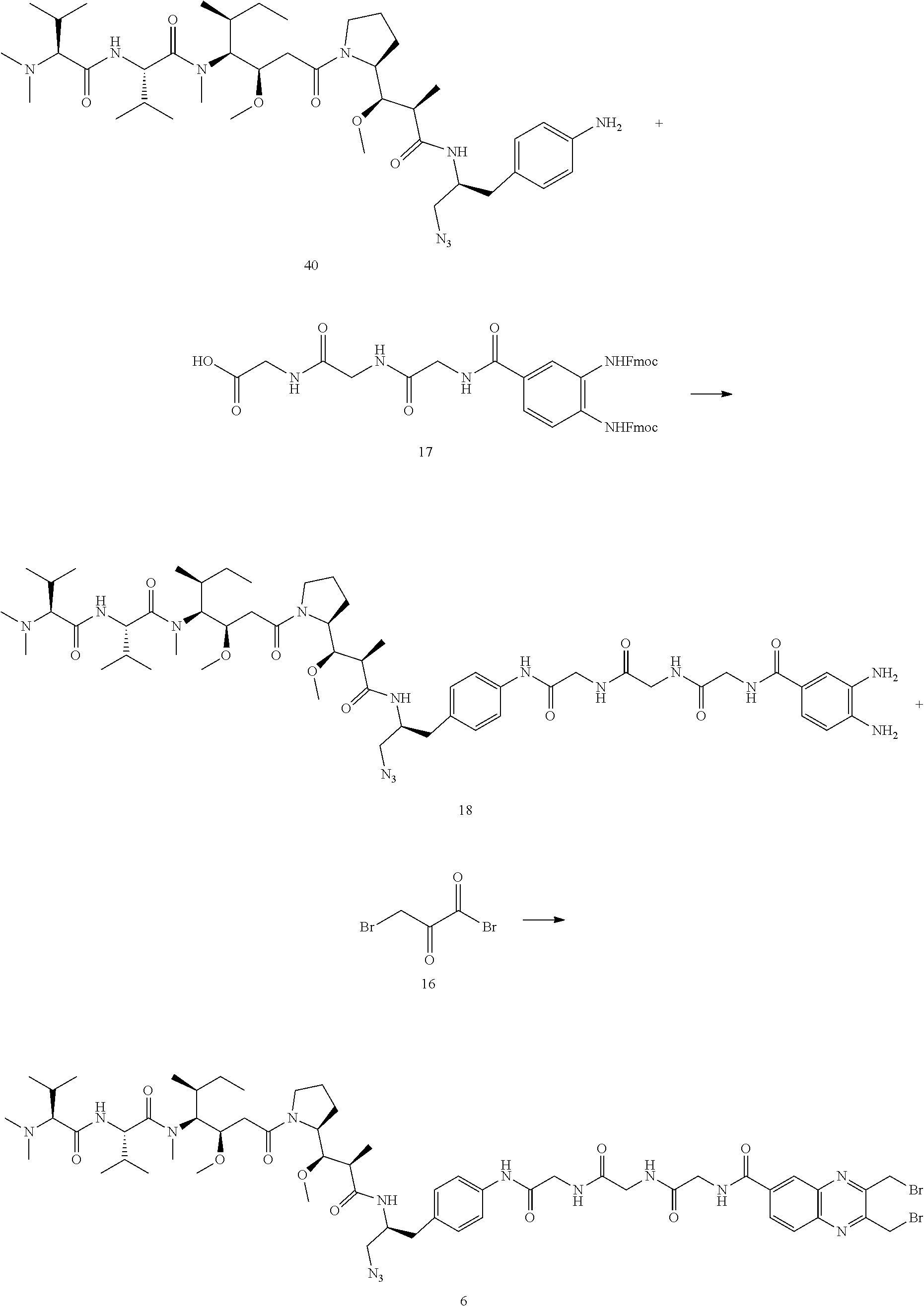

To compound 40 (TFA salt, 57 mg, 64.8 μmol) in 2 mL of DMF was added compound 17 (60 mg, 77.8 μmol), DIEA (34 μL, 0.2 mmol), and PyAOP (40 mg, 76.8 μmol). The mixture was stirred for 10 min, then 210 μL of piperidine was added and stirred for 20 min. The mixture was purified by HPLC to give compound 18 (65 mg). MS m/z 1077.7 (M+H).

To compound 18 (TFA salt, 43 mg, 33 μmol) in 2 mL of acetonitrile and 1 mL of water was added bromide 16 (17 mg, 70 μmol). The mixture was stirred for 10 min, then purified by HPLC to give compound 6 (37.1 mg). MS m/z 1226.6 (M+H).

### Example S7: Synthesis of Compound 7

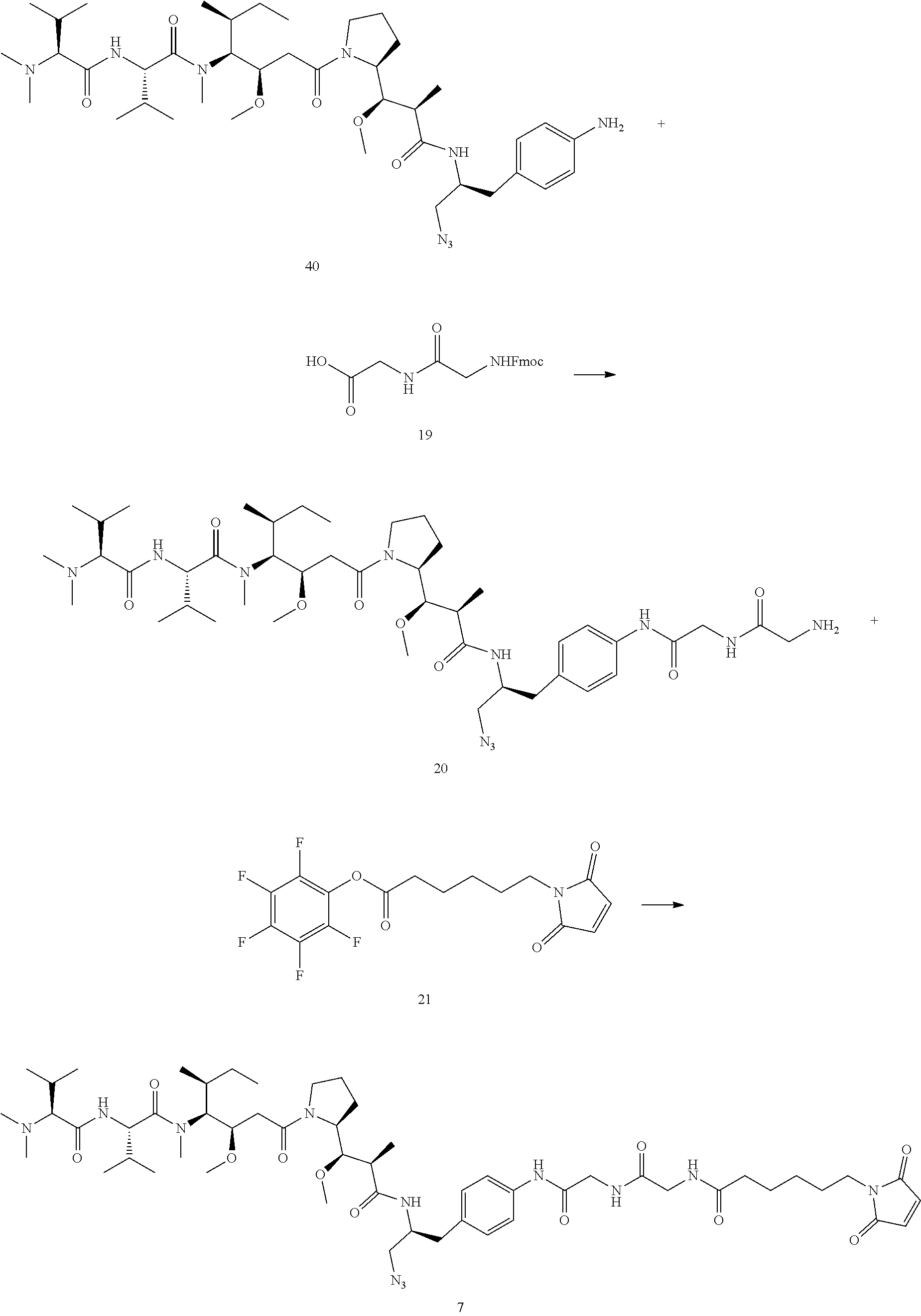

To compound 40 (TFA salt, 20 mg, 20 μmol) in 1 mL of DMF was added a solution of compound 19 (7.1 mg, 20 μmol), DIEA (13.9 μL, 80 μmol), and HATU (7.6 mg, 20 μmol) in 1 mL of DMF. The mixture was stirred for 16 h, then 20 μL of DBU was added and stirred for 20 min. The mixture was purified by HPLC to give compound 20 (16.9 mg). MS m/z 886.6 (M+H).

To compound 20 (TFA salt, 8.9 mg, 8 μmol) in 2 mL of DMF was added compound 21 (3.3 mg, 8.8 μmol). The mixture was stirred for 5 min, then purified by HPLC to give compound 7 (8.7 mg). MS m/z 1079.7 (M+H).

### Example S8: Synthesis of Compound 8

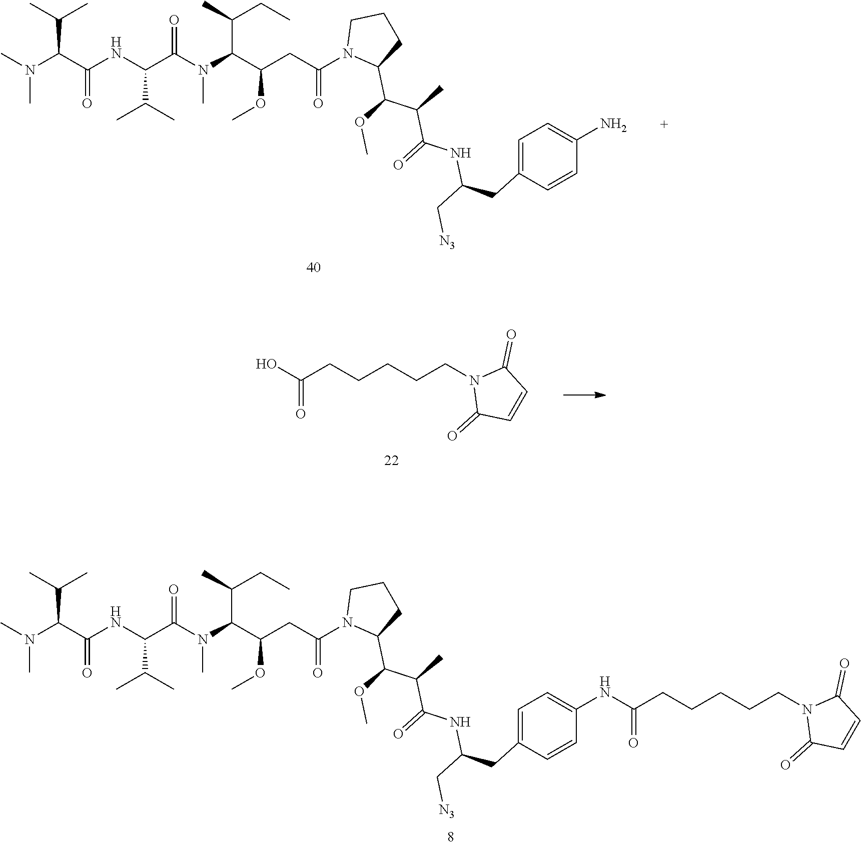 [SMILES: CC[C@H](C)[C@@H]([C@@H](CC(=O)N1CCC[C@H]1[C@H](OC)[C@@H](C)C(=O)N[C@H](CN=[N+]=[N-])Cc1ccc(N)cc1)OC)N(C)C(=O)[C@@H](NC(=O)[C@H](C(C)C)N(C)C)C(C)C ; O=C(O)CCCCCN1C(=O)C=CC1=O ; CC[C@H](C)[C@@H]([C@@H](CC(=O)N1CCC[C@H]1[C@H](OC)[C@@H](C)C(=O)N[C@H](CN=[N+]=[N-])Cc1ccc(NC(=O)CCCCCN2C(=O)C=CC2=O)cc1)OC)N(C)C(=O)[C@@H](NC(=O)[C@H](C(C)C)N(C)C)C(C)C]

To compound 40 (TFA salt, 57 mg, 64.8 μmol) in 2 mL of DMF was added acid 22 (16.4 mg, 77.8 μmol), DIEA (34 μL, 0.2 mmol), and PyAOP (40.5 mg, 77.7 μmol). The mixture was stirred for 10 min, then purified by HPLC to give compound 8 (55.5 mg). MS m/z 965.6 (M+H).

### Example S9: Synthesis of Compound 9

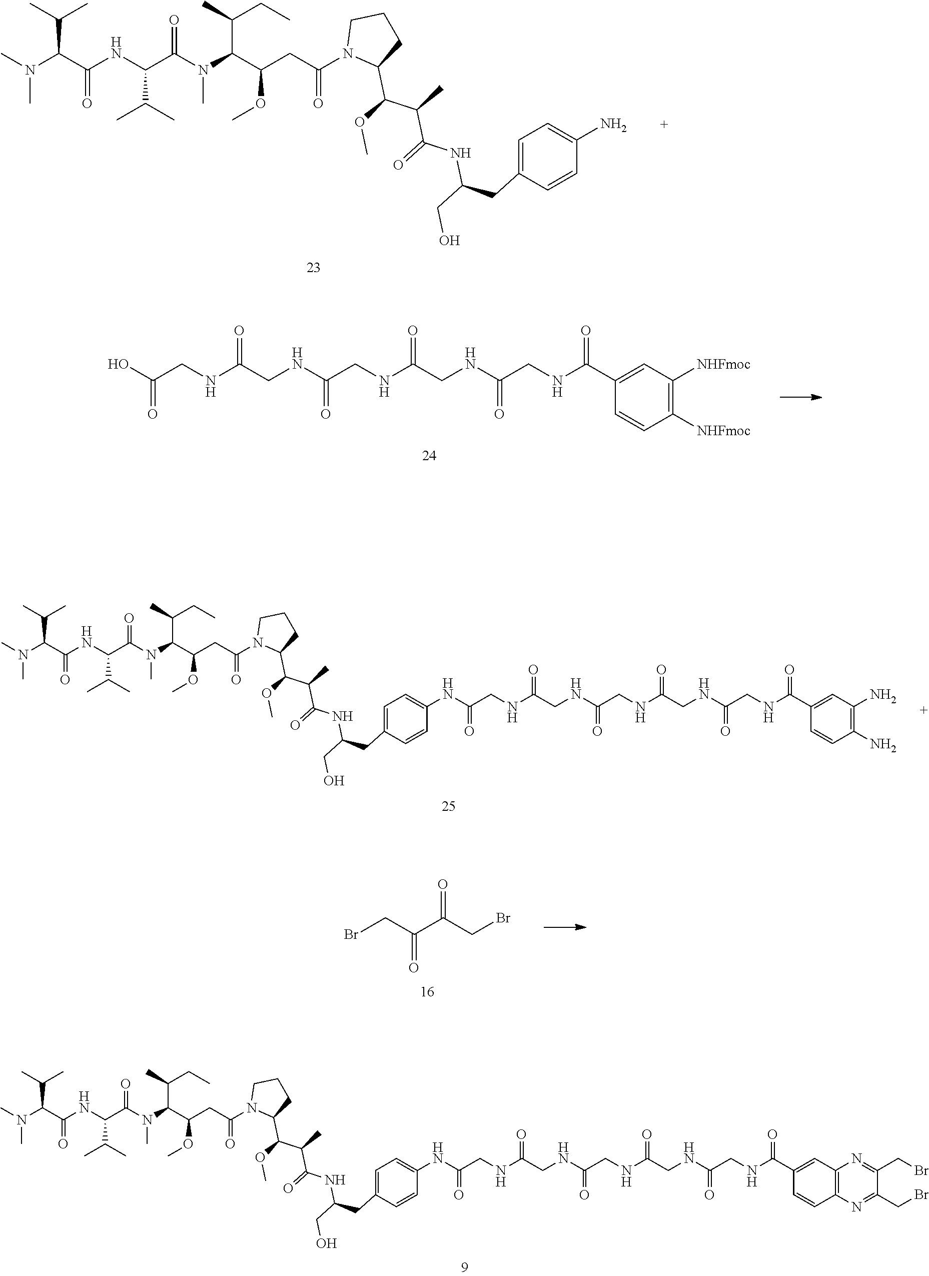

To compound 23 (TFA salt, 49 mg, 50 mmol) in 2 mL of DMF was added compound 24 (50 mg, 57 mmol), DIEA (34 mL, 0.2 mmol), and PyAOP (31 mg, 60 mmol). The mixture was stirred at room temperature for 10 min, then 200 μL of piperidine was added and stirred for 20 min. The mixture was purified by HPLC to give compound 25 (43 mg).

To compound 25 (TFA salt, 40 mg) in 2 mL of acetonitrile and 1 mL of water was added bromide 16 (17 mg, 70 mmol). The mixture was stirred for 10 min, then purified by HPLC to give compound 9 (32 mg). MS m/z 1373.5 (M+H).

### Example S10: Synthesis of Compound 50

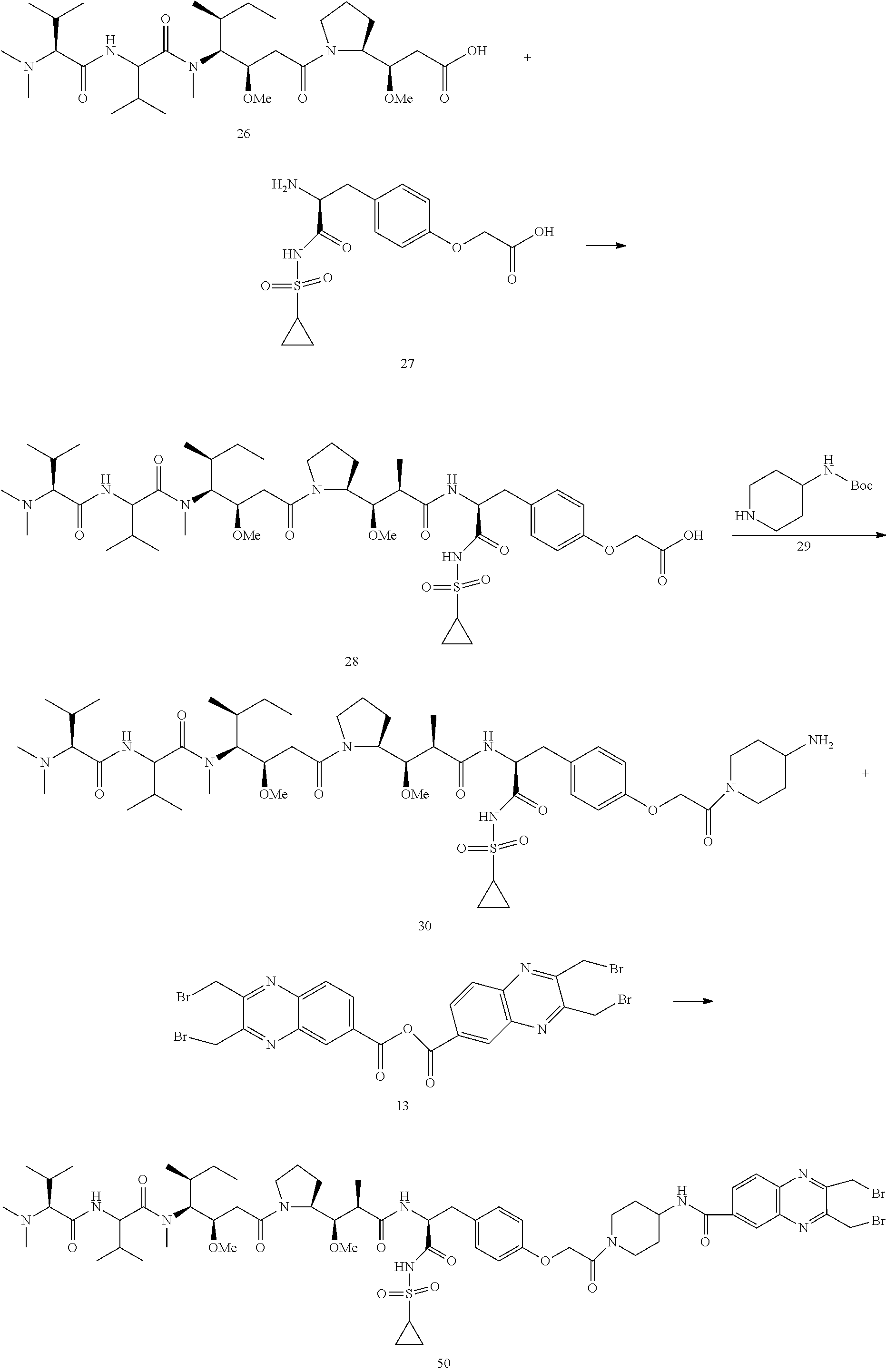

To TFA salt of compound 26 (100 mg, 0.14 mmol) in 2 mL of DMF was added HATU (53 mg, 0.14 mmol) and DIEA (22 mg, 0.19 mmol), stirred and followed by addition of compound 27 (64 mg, 0.14 mmol). The solution was stirred for 15 minutes. The reaction mixture was purified by HPLC to give compound 28 (67 mg) as a white powder. MS m/z 923.09 (M+H).

To a solution of compound 28 (TFA salt, 31 mg, 0.030 mmol), compound 29 (6.4 mg, 0.030 mmol) and HATU (11.4 mg, 0.030 mmol) in 2 mL of DMF was added DIEA (10 mg, 0.075 mmol). The mixture was stirred for 10 minutes and purified by (HPLC. The resulting white powder was treated with 30% TFA in DCM for 30 minutes and purified by HPLC to give compound 30 (34 mg) as a white powder. MS m/z 1020.3 (WE).

To a solution of compound 30 (TFA salt, 34 mg, 0.030 mmol) and compound 13 (32 mg, 0.045 mmol) in 2 mL DMF was added DIEA (10 mg, 0.075 mmol). The solution was stirred for 5 minutes and purified by HPLC to give compound 50 (16 g) as a white powder. MS m/z 1361.6 (M+H).

### Example S11: Synthesis of Compound 51

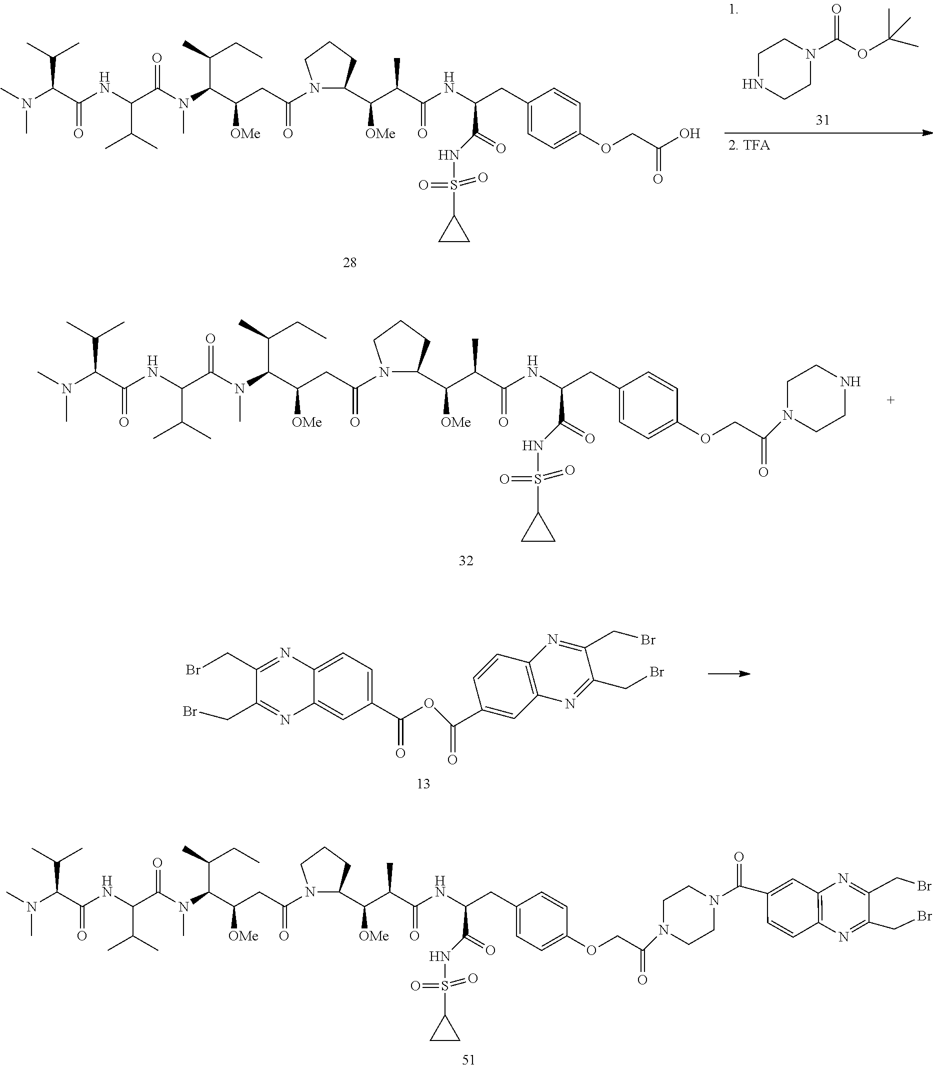

To a solution of TFA salt of compound 28 (30 mg, 0.029 mmol), compound 31 (5.4 mg, 0.029 mmol) and HATU (11 mg, 0.029 mmol) in 2 mL DMF was added DIEA (9.3 mg, 0.073 mmol). After being stirred for 10 minutes the mixture was purified by HPLC. The dried white powder was then treated with 50% solution of TFA in DCM for 2 hr. to give compound 32 (24 mg) as a white powder. MS m/z 992.1 (M+H).

To a solution of TFA salt of compound 32 (24 mg, 0.020 mmol) and compound 13 (21 mg, 0.029 mmol) in 2 mL DMF was added DIEA (10 mg, 0.075 mmol). The solution was stirred for 5 minutes and purified by HPLC to give compound 51 (12.3 mg) as a white powder. MS m/z 1333.7 (M+H).

### Example S12: Synthesis of Compound 52

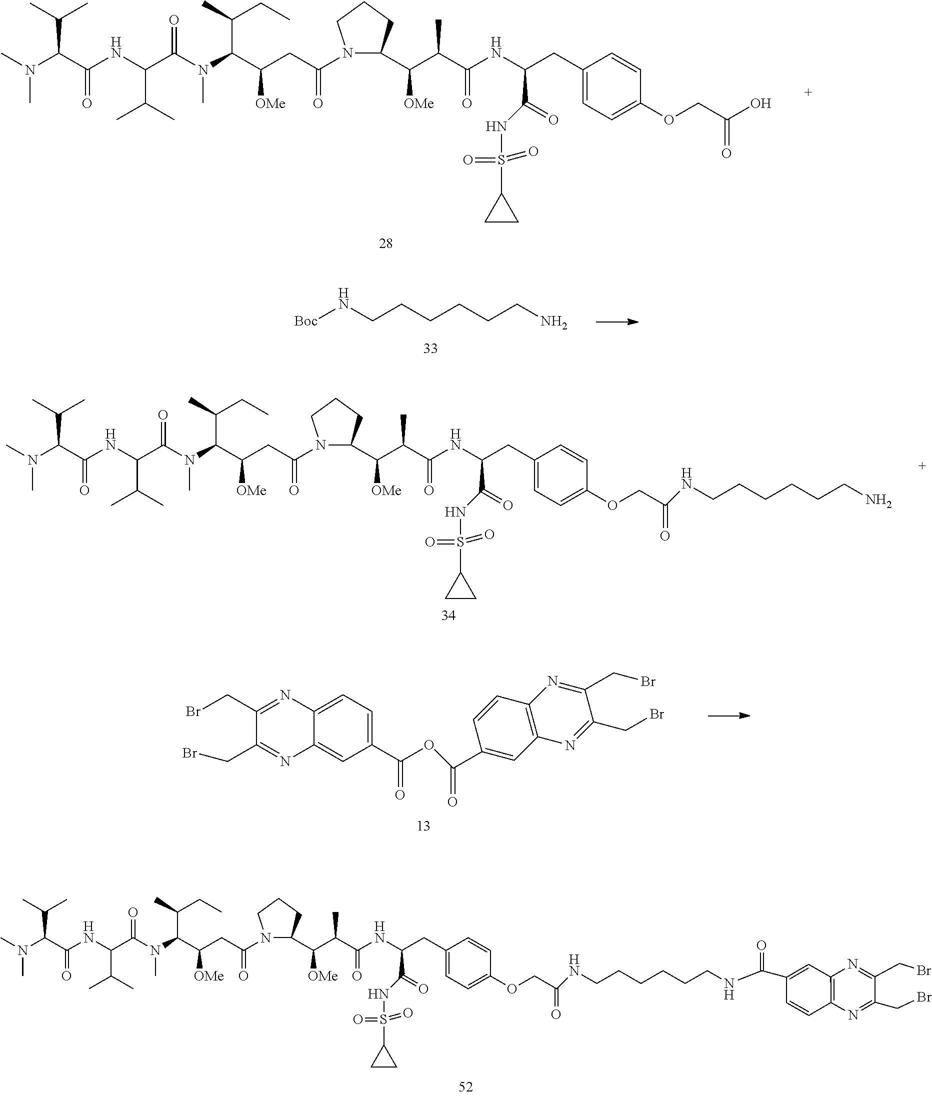

To a solution of TFA salt of compound 28 (50 mg, 0.048 mmol), compound 33 (11 mg, 0.051 mmol) and HATU (19 mg, 0.049 mmol) in 2 mL DMF was added DIEA (16 mg, 0.13 mmol). After being stirred for 10 minutes the mixture was purified by HPLC. The dried white powder was then treated with 30% solution of TFA in DCM for 1 hr. to give compound 34 (33 mg) as a white powder. MS m/z 1022.4 (M+H).

To a solution of TFA salt of compound 10 (33 mg, 0.029 mmol) and compound 13 (31 mg, 0.044 mmol) in 2 mL DMF was added DIEA (9 mg, 0.073 mmol). The solution was stirred for 5 minutes and purified by HPLC to give compound 52 (12.3 mg) as a white powder. MS m/z 1363.7 (M+H).

### Example S13: Synthesis of Compound 53

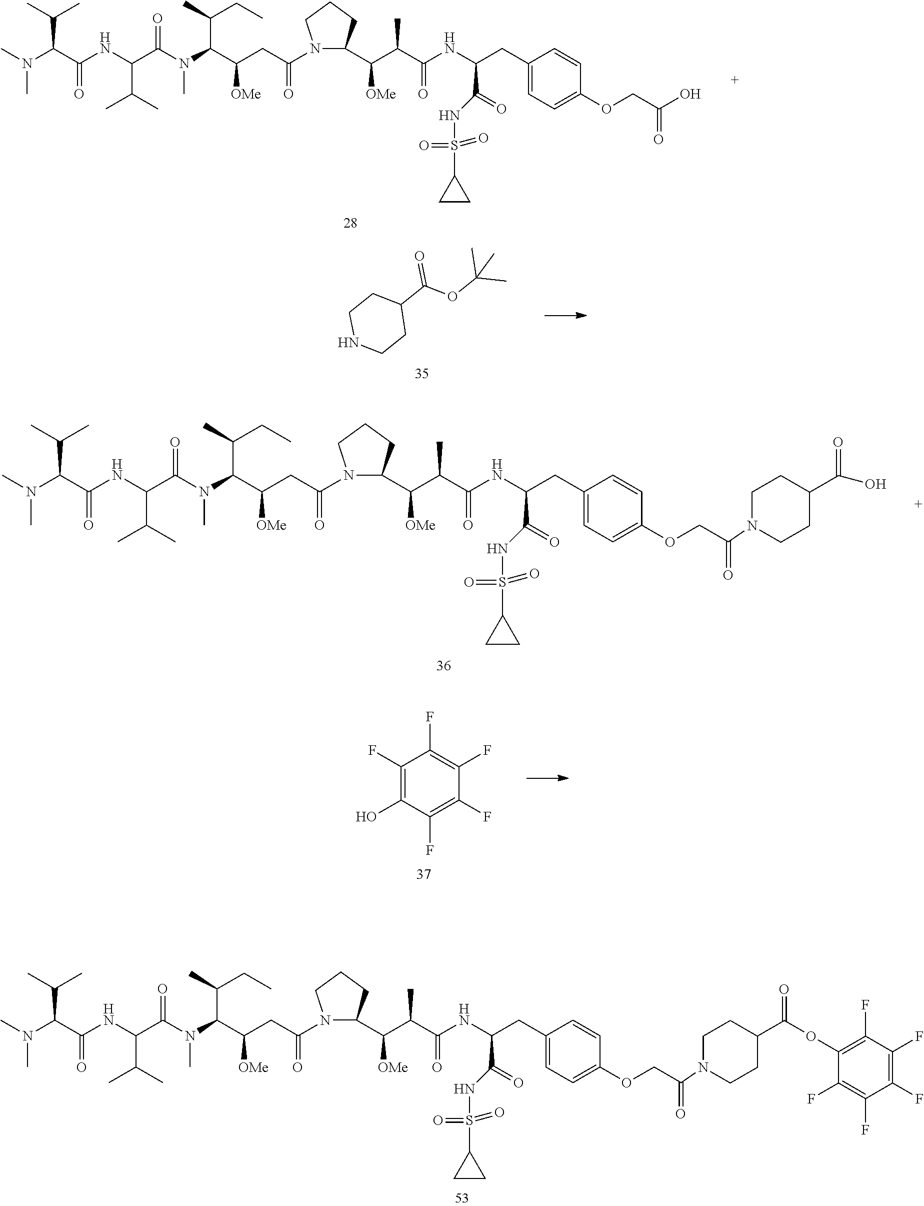

To a solution of TFA salt of compound 28 (78 mg, 0.076 mmol), compound 35 (14 mg, 0.076 mmol) and HATU (29 mg, 0.076 mmol) in 2 mL DMF was added DIEA (25 mg, 0.19 mmol). After being stirred for 10 minutes the mixture was purified by HPLC. The dried white powder was then treated with 50% solution of TFA in DCM for 2 hr. and purified by HPLC to give compound 36 (42 mg) as a white powder. MS m/z 1035.1 (M+H).

A solution of TFA salt of compound 36 (42 mg, 0.037 mmol), compound 37 (10 mg, 0.055 mmol) and EDC HCl (32 mg, 0.165 mmol) in 5 mL DCM was stirred for 2 hrs. and purified by HPLC to give compound 53 (13 mg) as a white powder. MS m/z 1200.8 (M+H).

Preparation of Antibody-Drug Conjugates (ADCs)

Antibody-Drug Conjugates (ADCs) were prepared by conjugating a compound 1-9 and 50-53 with either clone 1 (BCA7-2C₅ or AB1) or clone 2 (BCA7-2E1 or AB2) of anti-BCMA antibody. BCA7-2C₅ and BCA7-2E1 are human IgG1 antibodies. For general conjugation procedures no clone is indicated, for example, anti-BCMA-Compound 1 (also designated as anti-BCMA-1, BCMA-1, or ADC-1). In specific experiments, the clone is indicated, for example, anti-BCMA-AB1-Compound 1 (also designated as anti-BCMA-AB1-1, BCMA-AB1-1, or ADC-AB1-1).

### Example S14: Preparation of Antibody-Drug Conjugate (ADC) Anti-BCMA-Compound 1

The anti-BCMA antibody used in this Example has almost an identical antibody sequence of the BCA7-2C5 antibody described in WIPO publication No. WO 2020/176549. The heavy chain sequence of the anti-BCMA antibody used in this Example is identical to the heavy chain sequence of the BCA7-2C5 antibody described in WIPO publication No. WO 2020/176549. The light chain sequence of the anti-BCMA antibody used in this Example has one amino acid difference from the light chain of the BCA7-2C5 antibody described in WIPO publication No. WO 2020/176549. The third amino acid in the light chain of the BCA7-2C5 antibody described in WIPO publication No. WO 2020/176549 (SEQ ID NO:16) has been changed from Alanine to Valine (see SEQ ID NO: 7 in Table 2 above). Affinity purified anti-BCMA antibody was buffer exchanged into Conjugation Buffer (50 mM sodium phosphate buffer, pH 7.0-7.2, 4 mM EDTA) at a concentration of 5 mg/mL. To a portion of this antibody stock was added a freshly prepared 10 mM water solution of tris(2-carboxyethyl)phosphine) (TCEP) at 20-fold molar excess. The resulting mixture was incubated at 4-8° C. overnight. The excess TCEP was then removed by several rounds of centrifugal filtration with fresh Conjugation Buffer. UV-Vis quantification of recovered, reduced antibody material was followed by confirmation of sufficient free thiol-to-antibody ratio (SH/Ab). Briefly, a 1 mM aliquot of freshly prepared Ellman's Reagent (5,5′-dithiobis-(2-nitrobenzoic acid) in Conjugation Buffer was mixed with an equal volume of purified antibody solution. The resulting absorbance at 412 nm was measured and the reduced cysteine content was determined using the extinction coefficient of 14,150 M⁻¹ cm⁻¹. Under these conditions, SH/Ab ratio measured -6.

To initiate conjugation of toxin-linker material to anti-BCMA antibody, compound 1 was freshly dissolved in a 3:2 acetonitrile/water mixture to a concentration of 5 mM. Propylene glycol was then added to a portion of the reduced, purified (TCEP removed) anti-BCMA antibody to give a final concentration of 30% (v/v) propylene glycol immediately prior to addition of 1 in 4.5-fold molar excess. After thorough mixing and incubation at ambient temperature for 2 h, the crude conjugation reaction was analyzed by HIC-HPLC chromatography to confirm reaction completion (disappearance of starting antibody peak) at 280 nm wavelength detection. Purification of the resulting ADC-1 conjugate was then carried out by gel-filtration chromatography using an AKTA system equipped with a Superdex 200 pg column (GE Healthcare) equilibrated with PBS. The average drug-to-antibody ratio (DAR) was calculated to be 3.8 based on comparative peak area integration of the HIC-HPLC chromatogram. Confirmation of low percent (<5%) high molecular weight (HMW) aggregates for the resulting ADC-1 was determined using analytical SEC-HPLC.

### Example S15: Preparation of Antibody-Drug Conjugates (ADCs) Anti-BCMA-Compound 2, Anti- BCMA-Compound 3, Anti-BCMA-Compound 4, Anti-BCMA-Compound 5, Anti-BCMA-Compound 6, Anti-BCMA-Compound 9, Anti-BCMA-Compound 50, Anti-BCMA-Compound 51 and Anti-BCMA-Compound 52

The additional ADCs anti-BCMA-Compound 2, anti-BCMA-Compound 3, anti-BCMA-Compound 4, anti-BCMA-Compound 5, anti-BCMA-Compound 6, anti-BCMA-Compound 9, anti-BCMA-Compound 50, anti-BCMA-Compound 51 and anti-BCMA-Compound 52 were prepared as outlined in Example S14 using compounds 2, 3, 4, 5, 6, 9, 50, 51, or 52, respectively, in place of 1. According to HIC-HPLC analysis, the resulting average DAR for ADC-52 was 3.5, DAR for ADC-2 and ADC-50 was 3.4, and DAR for ADCs 3-6 and 51 was 3.2-3.3.

### Example S16: Preparation of Antibody-Drug Conjugate (ADC) Anti-BCMA-Compound 7

Affinity purified anti-BCMA antibody was buffer exchanged into Conjugation Buffer at a concentration of 5 mg/mL. To a portion of this antibody stock was added a freshly prepared 10 mM water solution of tris(2-carboxyethyl)phosphine) (TCEP) at 2.5-fold molar excess. The resulting mixture was incubated at 37° C. for 2 h. After freshly dissolving Compound 7 in anhydrous dimethylsulfoxide (DMSO) to 5 mM, a portion of this mixture was added to the reduced anti-BCMA antibody solution in 5-fold molar excess. After thorough mixing and incubation at ambient temperature for 2 h, the crude conjugation reaction was analyzed by HIC-HPLC chromatography to confirm reaction completion (disappearance of starting antibody peak) at 280 nm wavelength detection. Purification and analysis of the resulting ADC-7 conjugate proceeded in a manner identical to ADC-1-6 conjugates. The resulting average DAR for ADC-7 was 4.0 according to HIC-HPLC analysis.

### Example S17: Preparation of Antibody-Drug Conjugate (ADC) Anti-BCMA-Compound 8

The ADC anti-BCM A-Compound 8 was prepared as outlined in Example S10 using Compound 8, in place of 1. According to HIC-HPLC analysis, the resulting average DAR for ADC-8 was 3.6.

### Example S18: Preparation of Antibody-Drug Conjugate (ADC) Anti-BCMA-Compound 53

To a solution of 0.5-50 mgs/mL of antibody in buffer at pH 6.0-9.0 with 0-30% organic solvent, was added 0.1-10 eq of activated drug linker conjugate 53 in a manner of portion wise or continuous flow. The reaction was performed at 0-40° C. for 0.5-50 hours with gentle stirring or shaking, monitored by HIC-HPLC. The resultant crude ADC product underwent necessary down-stream steps of desalt, buffer changes/formulation, and optionally, purification. Purification and analysis of the resulting ADC-53 conjugate proceeded in a manner identical to ADC-1-6 conjugates. The resulting average DAR for ADC-53 was 1.5 according to HIC-HPLC analysis.

### BIOLOGICAL EXAMPLES

In vitro and in vivo Efficacy of Antibody-Drug Conjugates (ADCs) was assessed using two different clones of anti-BCMA antibody—BCA7-2C5 (AB1) and BCA7-2E1 (AB2).

### Example B1: In Vitro Efficacy of Antibody-Drug Conjugates (ADCs) Anti-BCMA-Compound 1 (Anti-BCMA-1), Anti-BCMA-Compound 2 (Anti-BCMA-2), Anti-BCMA-Compound 3 (Anti-BCMA-3), Anti-BCMA-Compound 4 (Anti-BCMA-4), Anti-BCMA-Compound 5 (Anti-BCMA-5), Anti-BCMA-Compound 6 (Anti-BCMA-6), Anti-BCMA-Compound 7 (Anti-BCMA-7), and Anti-BCMA-Compound 8 (Anti-BCMA-8)

The in vitro efficacies of ADCs anti-BCMA-Compound 1, anti-BCMA-Compound 2, anti-BCMA-Compound 3, anti-BCMA-Compound 4, anti-BCMA-Compound 5, anti-BCMA-Compound 6, anti-BCMA-Compound 7, and anti-BCMA-Compound 8, were evaluated using the following human cancer cell lines: K562, MM.1R and NCI-H929, purchased from the American Type Culture Collection (ATCC; Manassas, VA). The cells were cultured in RPMI-1640 medium (Gibco ThermoFisher; Waltham, MA) supplemented with 10% heat-inactivated fetal bovine serum (FBS: Corning; Corning, NY, USA) and 1× penicillin-streptomycin (Corning) and maintained at 37° C. in a 5% CO₂ humidified environment.

The in vitro assays were performed as follows: Tumor cells were harvested by centrifugation at 300 g for 5 minutes and plated into 96-well clear bottom white-walled plates (5,000 to 10,000 cells/well in 50 μL complete medium) and maintained at 37° C. Cells were then treated in duplicate with 50 μL of test articles prepared at 2× final concentration that were serially diluted in complete medium and incubated at 37° C. for up to 120 hrs. After treatment, inhibition of cancer cell growth was determined using the Cell Titer-Glo® 2.0 Cell Viability Assay (Promega; Madison, WI, USA) as described by the manufacturers' protocol. Luminescence was measured using a Perkin-Elmer Envision 2104 Microplate Reader (Waltham, MA).

Data were normalized to non-treated controls using Microsoft Excel (Redmond, WA, USA) and analyzed using GraphPad Prism software (version 8; La Jolla, CA, USA). Half-maximal effective concentrations (EC₅₀) were derived from dose response curves were generated using non-linear regression analysis fit to a 4-parameter logistic equation.

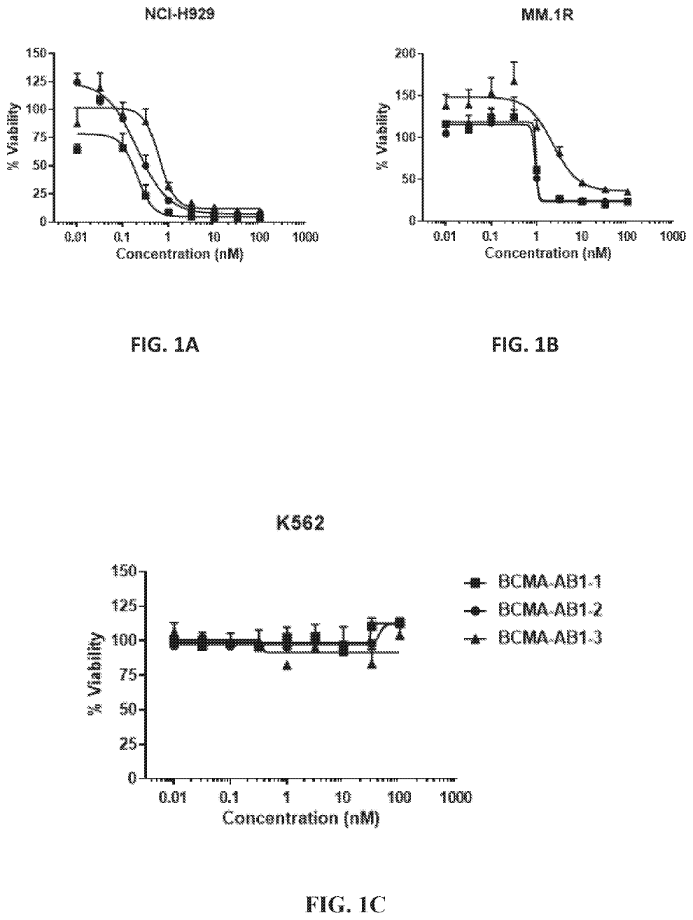

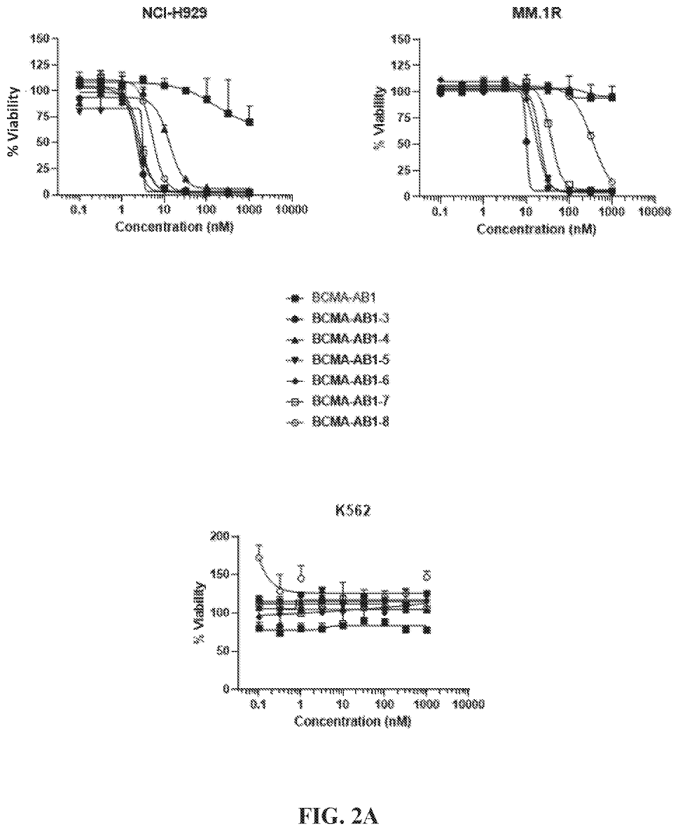

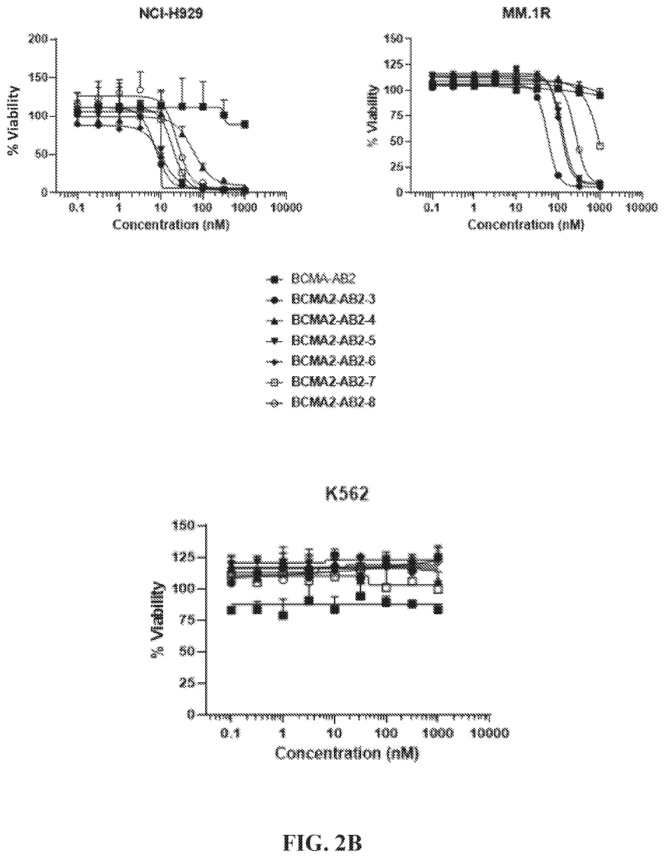

Cell viability, for anti-BCMA-1, anti-BCMA-2, anti-BCMA-3, anti-BCMA-4, anti-BCMA-5, anti-BCMA-6, anti-BCMA-7, and anti-BCMA-8 is shown in FIGS. 1-2, and Tables 4-6.

In vitro cytotoxic activities and targeting specificity of the ADCs described herein were evaluated against BCMA-expressing NCI-H929 and MM.1R, and BCMA-negative K562 cancer cell lines using standard cell viability assays. As shown in FIG. 1, anti-BCMA-AB1-1 to -3 (where AB is BCA7-2C₅ clone of BCMA and AB2 is BCA7-2E1 clone of BCMA) dose-dependently reduced NCI-H929 and MM.1R cell viability and did not show activity against K562 cells in 5-day assays. A range in potency as determined by EC50 of ˜0.2 to 2 nM against BCMA-expressing cell lines were observed among the ADCs with different conjugation chemistries to the anti-BCMA antibody (Table 4).

Although ADC-1 and ADC-2 have identical structures, they have different EC₅₀ values, likely due to the differences in their DAR values. ADC-1 exhibited a lower DAR value than ADC-2 likely due to higher reactivity of compound 1 with the antibody compared to compound 2. Without being bound by a particular theory, it is possible that because there are more payloads per antibody in ADC-1 compared to ADC-2, ADC-1 is more potent (i.e., has lower EC₅₀).

Summary of EC₅₀ Values (nM) of anti-BCMA-AB1-1 to -3 in Human Tumor Cells is presented in Table 4.

TABLE 4
EC₅₀ Values (nM) of anti-BCMA-AB1-Compound
1 to -Compound 3 in Human Tumor Cells

| | Cell Line | | |
|---|---|---|---|
| Test Article | NCI-H929 | MM.1R | K562 |
| BCMA-AB1-1 | 0.1679 | 1.01937 | >100 |
| BCMA-AB1-2 | 0.3162 | 0.9873 | >100 |
| BCMA-AB1-3 | 0.6989 | 2.282 | >100 |

The effect of Gly peptide linker length and conjugation chemistry to the anti-BCMA antibody on ADC activity was evaluated amongst anti-BCMA-AB1-3 through -8 in 4-day assays. All C-lock conjugated ADCs exhibited dose-dependent cell killing of NCI-H929 and MM.1R (FIG. 2A). In the more sensitive NCI-H929 cell line, the anti-BCMA ADC lacking a Gly linker (anti-BCMA-AB1-4) exhibited two-digit nanomolar potency (EC₅₀=13.14 nM) compared to single-digit nanomolar potency (EC₅₀˜2-5 nM) for ADCs containing a Gly2, Gly3 or Gly4 (anti-BCMA-AB1-5, -6, and -3, respectively) linker (Table 5).

Summary of EC₅₀ values (nM) of anti-BCMA-AB1-Compound 3 to -Compound 8 in Human Tumor Cells is presented in Table 5.

TABLE 5
EC₅₀ Values (nM) of anti-BCMA-AB1-3
to -8 in Human Tumor Cells

| | Cell Line | | |
|---|---|---|---|
| Test Article | NCI-H929 | MM.1R | K562 |
| BCMA-AB1 | >1000 | >1000 | >1000 |
| BCMA-AB1-3 | 2.206 | 10.0 | >1000 |
| BCMA-AB1-4 | 13.14 | >1000 | >1000 |
| BCMA-AB1-5 | 3.041 | 20.48 | >1000 |
| BCMA-AB1-6 | 2.383 | 16.58 | >1000 |
| BCMA-AB1-7 | 2.643 | 37.74 | >1000 |
| BCMA-AB1-8 | 5.150 | 348.2 | >1000 |

The lack of a Gly linker of the maleimide-conjugated anti-BCMA-AB1-8 ADC resulted in comparable activity to the Gly2 peptide linker ADC, anti-BCMA-AB1-7, in NCI-H929 cells. As anticipated, appreciable cytotoxic activity of the unconjugated anti-BCMA antibody was not observed against any cell line therefore indicating that the cell-killing effects of the anti-BCMA ADCs are driven by the presence of the small molecule payload. Similar trends were also observed with conjugation of Compounds 3-8 to a different anti-BCMA antibody clone (where AB2 is BCA7-2E1), anti-BCMA-AB2 (FIG. 2B and Table 6).

The anti-BCMA antibody used in this Example has the antibody sequence of the BCA7-2E1 antibody described in WIPO publication No. WO 2020/176549. Summary of EC₅₀ values (nM) of anti-BCMA-AB2-Compound 3 to -Compound 8 in Human Tumor Cells is presented in Table 6.

TABLE 6
EC₅₀ Values (nM) of anti-BCMA-AB2-3
to -8 in Human Tumor Cells

| | Cell Line | | |
|---|---|---|---|
| Test Article | NCI-H929 | MM.1R | K562 |
| BCMA-AB2 | >1000 | >1000 | >1000 |
| BCMA-AB2-3 | 7.312 | 55.34 | >1000 |
| BCMA-AB2-4 | 59.01 | >1000 | >1000 |
| BCMA-AB2-5 | 10.03 | 142.7 | >1000 |
| BCMA-AB2-6 | 9.44 | 129.3 | >1000 |
| BCMA-AB2-7 | 19.43 | 259.4 | >1000 |
| BCMA-AB2-8 | 29.72 | >1000 | >1000 |

Comparison of Table 5 and Table 6 shows that ADCs of compounds 3-8 conjugated with anti-BCMA-AB1 clone were more potent than ADCs of compounds 3-8 conjugated with anti-BCMA-AB2 clone. The most potent ADC is the one with Compound 3, ADC-3. BCMA-AB1-3 was the leading ADC selected from ADCs 1-9.

### Example B2: In Vitro Efficacy of Antibody-Drug Conjugates (ADCs) Anti-BCMA-Compound 50 (ADC-50), Anti-BCMA-Compound 51 (ADC-51), Anti-BCMA-Compound 52 (ADC-52), Anti-BCMA-Compound 53 (ADC-53) and Anti-BCMA-Compound 3 (ADC-3)

The in vitro efficacies of ADCs anti-BCMA-Compound 50, anti-BCMA-Compound 51, anti-BCMA-Compound 52, and anti-BCMA-Compound 53, were compared to anti-BCMA-Compound 3. Anti-BCMA-AB1 clone was conjugated with compounds 50, 51, 52, 53 or 3 and the ADCs were evaluated using the following human cancer cell lines: BCMA-negative K562 and BCMA-positive NCI-H929, purchased from the American Type Culture Collection (ATCC; Manassas, VA).

Cell Culture Method: The cell lines were cultured in RPMI-1640 medium (Catalog #10-041-CV: Corning) supplemented with 10-20% fetal bovine serum (FBS; Catalog #MT35011CV; Corning) and 1× penicillin-streptomycin (Catalog #30-002-Cl, Corning) and maintained at 37° C. in a 5% C₀₂ humidified environment. Viable cell counts were made by Trypan blue exclusion using a Countess or Countess II automated cell counter.

The in vitro assays were performed as follows: All cells were harvested by removal of a portion of the cell culture suspension followed by centrifugation at 300 g for 5 minutes, followed by resuspension in cell culture medium (described above in Cell Culture method), viable cell count (as described above in Cell Culture method), and then seeded into 384-well white wall clear bottom plates (Catalog #3765, Corning) at a density of 2,500 cells/well in growth media. Plates were maintained at 37° C. The outer wells of plates contained medium only and were used for background subtraction for the cell viability assay. Working solutions of test articles were prepared at 2× final concentrations with 5-fold serial dilutions in complete growth medium. Cell treatment was performed in either technical triplicates or duplicates and maintained at 37° C. for 120-hour assay. After treatment, cell viability was determined by CellTiter-Glo 2.0 assay (Catalog #G9243; Promega; Madison, WI, USA) based on the manufacturer's instructions. CellTiter Glo reagent reacts with ATP in metabolically active cells to give a luminescent readout that is directly proportional to the number of viable cells. Briefly, plates were removed from the incubator and equilibrated to room temperature before addition of CellTiter Glo reagent. Luminescence was measured using a Tecan Spark microplate reader (Tecan; Mannedorf, Switzerland).

Data Analysis: For Cytotoxicity assays, raw luminescence data was background subtracted with average luminescence from the outer wells containing medium only and normalized to untreated controls using Excel (Microsoft; Albuquerque, NM). Dose-response relationships and EC50 values were determined based on non-linear regression analysis of normalized data fit to a four-parameter logistic equation using GraphPad Prism 8.0.

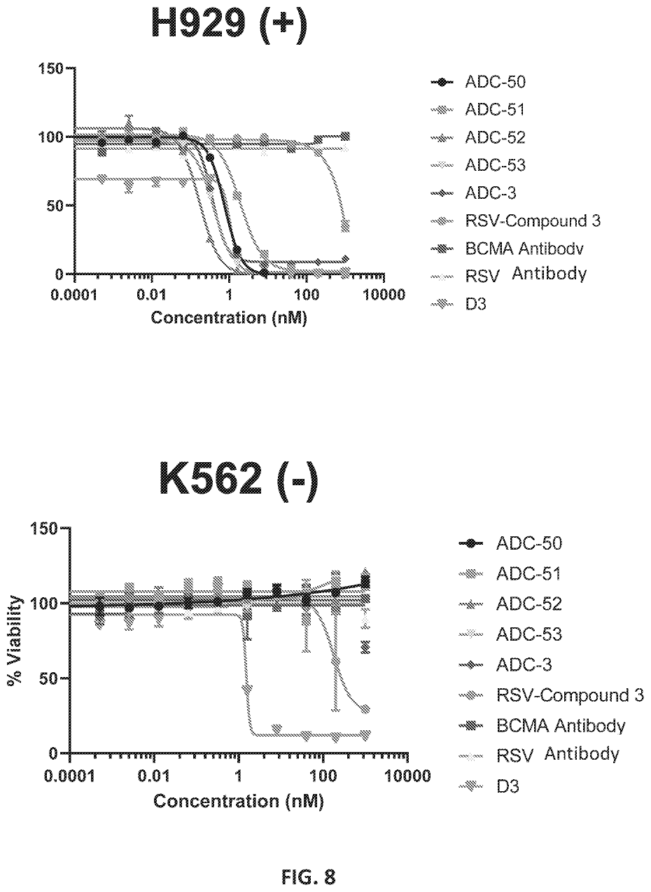

Cell viability, for ADC-50, ADC-51, ADC-52, ADC-53, ADC-3, and controls (RSV-Compound 3, BCMA antibody, RSV antibody, and D3) is shown in FIG. 8, and Table 7. Where ADC-50, ADC-51, ADC-52, ADC-53, and ADC-3 are ADCs of anti-BCMA-AB1 clone.

In vitro cytotoxic activities and targeting specificity of the ADCs described herein were evaluated against BCMA-positive NCI-H929 and BCMA-negative K562 cancer cell lines using standard cell viability assays. As shown in FIG. 8, treatment with ADC-50, ADC-51, ADC-52, ADC-53, and ADC-3 (where BCA7-2C₅ clone of BCMA was used) dose-dependently reduced NCI-H929 cell viability and did not show activity against K562 cells in 5-day assays. A range in potency as determined by EC50 of -0.17 to 1.9 nM against BCMA-positive NCI-H929 cell lines was observed (Table 7).

Comparison of Conjugation chemistry between ADC-53 (PFP linkage) and ADC-50 (C-lock linkage) revealed more than 2-fold decrease in potency from EC₅₀ 0.398 nM to 0.76 nM, respectively, despite the lower DAR of ADC-53 (DAR ˜1.5) compared to ADC-50 (DAR ˜3.4). Comparison of linker chemistry among C-lock conjugations (ADC-50, ADC-51, ADC-52), revealed single digit nanomolar (ADC-51) and sub-nanomolar (ADC-50, ADC-52) EC₅₀. Comparison of ADC-3 with ADC-50, ADC-51, and ADC-52, revealed ADC-52 showed ˜2-fold enhanced potency compared to ADC-3. Isotype control RSV-Compound 3 was >500× less active compared to BCMA targeting ADCs indicating that cytotoxicity was driven by BCMA targeting. In BCMA-negative K562 cells, neither antibody nor any BCMA targeting ADC showed cytotoxicity at concentrations up to 1 μM (FIG. 8). In contrast, D3 payload alone inhibited cell proliferation across all cell lines in a dose-dependent manner with an average EC₅₀ 0.88-1.53 nM, regardless of BCMA expression level, indicating that the cell-killing effects of the anti-BCMA ADCs are driven by the presence of the small molecule payload.

Summary of EC₅₀ Values (nM) of anti-BCMA-AB1-Compound 50 to -Compound 53 and anti-BCMA-AB1-Compound 3 in Human Tumor Cells is presented in Table 7.

TABLE 7
EC₅₀ Values (nM) of anti-BCMA-AB1-Compound 50 to -Compound
53 and anti-BCMA-AB1-Compound 3 in Human Tumor Cells

| Sample | H929 (+) | K562 (−) |
|---|---|---|
| ADC-50 | 0.7620 | >1000 |
| ADC-51 | 1.928 | >1000 |
| ADC-52 | 0.169 | >1000 |
| ADC-53 | 0.3977 | >1000 |
| ADC-3 | 0.3683 | >1000 |
| RSV-Compound 3 | >1000 | 178.300 |
| BCMA antibody | >1000 | >1000 |
| RSV Antibody | >1000 | >1000 |
| D3 | 1.079 | 1.523 |

### Example B3: In Vivo Efficacy of Antibody-Drug Conjugates (ADCs) Anti-BCMA-Compound 3

Female CB17 SCID beige mice, 6 weeks of age, were purchased from Charles River Laboratories.

Human multiple myeloma tumor cell lines NCI-H929 and OPM2 were cultured and expanded in RPMI 1640 medium supplemented with 10% FBS, 100 units/ml of penicillin and 100 μg/ml of streptomycin at 37° C. in a 5% CO₂ humidified environment for a period of 2-3 weeks before harvesting for implantation. Cell viability determined by Trypan blue dye exclusion assay was >90% before implantation. 5 million of OPM2 or NCI-H929 cells in 100 μl of PBS—Matrigel 1:1 (v/v) mixture were inoculated to the right upper flank of each mouse by s.c. injection.

Tumor volume measurement was started at day 14 after tumor cell inoculation and performed twice weekly. The longest longitudinal diameter as length and the widest transverse diameter as width were measured by using a digital caliper. Tumor volume (TV) were then calculated by the formula: TV=[length×(width)²]/2 and were analyzed in Excel.

The treatment was started when average tumor size reaches ˜400, 200 and 150 mm³ for NCI-H929 tumor xenografts in Experiment I, III and IV, respectively, or ˜240 mm³ for OPM2 tumor xenograft in Experiment I.

Mice were euthanized when tumor size reached 2000 mm³.

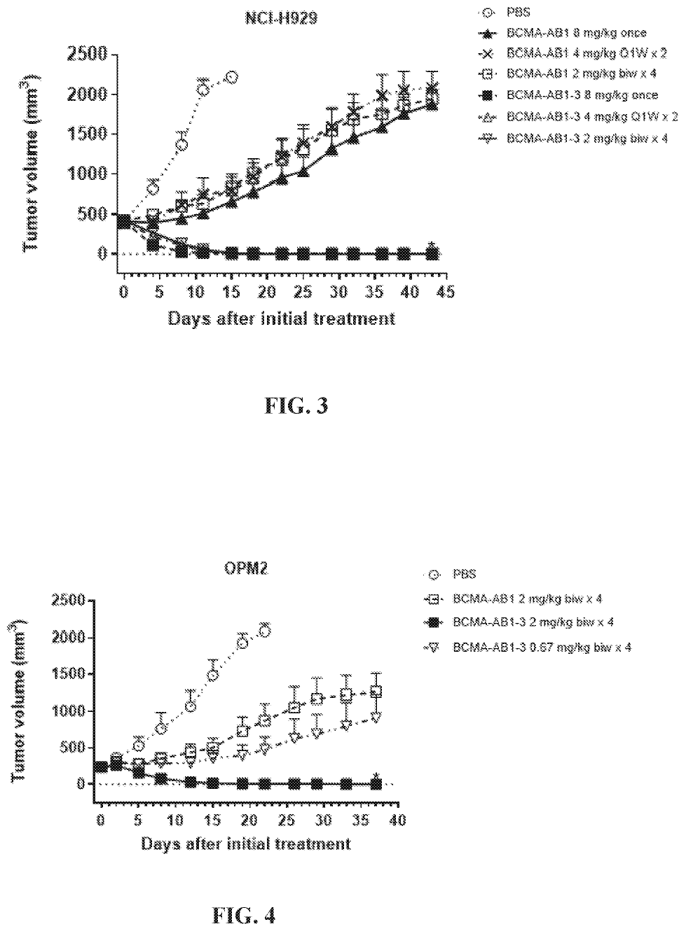

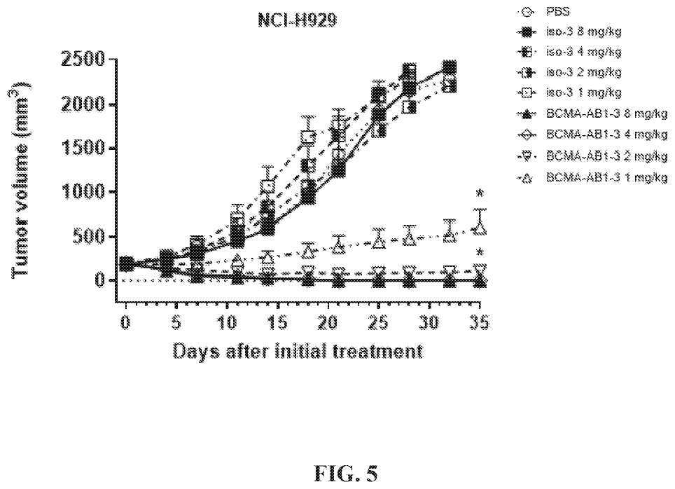

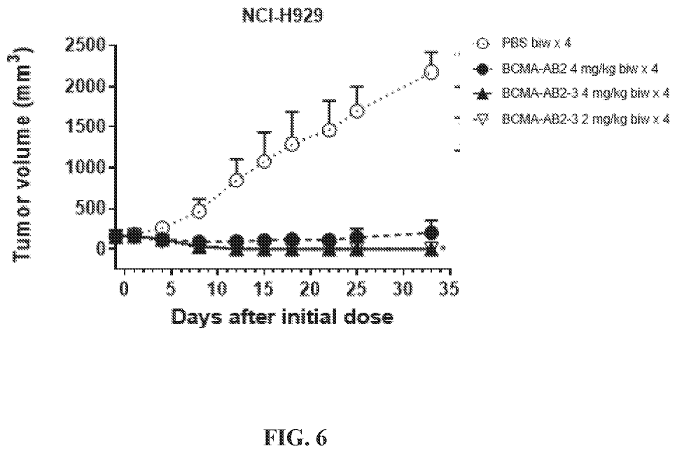

After tumor-bearing mice were randomized, anti-BCMA antibody (BCMA-AB1 or BCMA-AB2), anti-BCMA antibody conjugate with Compound 3 (BCMA-AB1-3 or BCMA-AB2-3) or iso-type antibody conjugate with Compound 3 (iso-3) diluted in PBS were administered to mice through i.p. injections. Treatment regimens included 8 mg/kg once, 4 mg/kg Q1W×2 and 2 mg/kg biw×4 of anti-BCMA-AB1 or anti-BCMA-AB1-Compound 3 in Experiment I (FIG. 3); 2 mg/kg biw×4 and 0.67 mg/kg biw×4 of anti-BCMA-AB1 or anti-BCMA-AB1-Compound 3 in Experiment II (FIG. 4); 1, 2, 4, and 8 mg/kg once of anti-BCMA-AB1-Compound 3 or iso-3 in Experiment III (FIG. 5); and 4 mg/kg biw×4 of anti-BCMA-AB2 or 2 and 4 mg/kg biw×4 of anti-BCMA-AB2-Compound 3 in Experiment IV (FIG. 6).

Raw data of tumor measurements were analyzed in Excel. Tumor growth curves were plotted using GraphPad Prism 8.0 software and values were presented as mean t SEM.

In NCI-H929 tumor xenograft model of Experiment I (as shown in FIG. 3), all treatment regimens of anti-BCMA-AB1-Compound 3 (BCMA-AB1-3) significantly inhibited tumor growth and were much better than those of anti-BCMA-AB1 (p<0.0001, anti-BCMA-AB1-3 vs PBS; p<0.0001, BCMA-AB1-3 vs BCMA-AB1; two-way ANOVA with Tukey's test on tumor volumes at end points). Although BCMA-AB1 slowed down tumor growth, the tumors could still reach average sizes close to that of PBS control group. All regimens of BCMA-AB1-3 induced dramatic and prolonged tumor regressions and eliminated all tumors in about two weeks after initial treatments.

In OPM2 tumor xenograft model of Experiment II (as shown in FIG. 4), BCMA-AB1-3 significantly and dose-dependently inhibited tumor growth and was much better than BCMA-AB1 (p<0.0001, BCMA-AB1-3 vs PBS; p<0.001, BCMA-AB1-3 2 mg/kg vs BCMA-AB1-3 0.67 mg/kg; p<0.001, BCMA-AB1-3 2 mg/kg vs BCMA-AB1 2 mg/kg; two-way ANOVA with Tukey's test on tumor volumes at end points). The high-dose regimen of BCMA-AB1-3 also induced dramatic and prolonged tumor regressions and eliminated all tumors in about two weeks after initial treatments.

In NCI-H929 tumor xenograft model of Experiment III (as shown in FIG. 5), single-dose regimens of BCMA-AB1-3 significantly inhibited tumor growth in a dose-dependent manner, but iso-3 did not have efficacy (p<0.0001, BCMA-AB1-3 vs PBS; p<−0.0001, BCMA-AB1-3 vs iso-3; two-way ANOVA with Tukey's test on tumor volumes at end points). High doses of BCMA-AB1-3 induced dramatic and prolonged tumor regressions and eliminated all tumors in about two weeks after initial treatments.

In NCI-H929 tumor xenograft model of Experiment IV (as shown in FIG. 6), both 2 and 4 mg/kg treat regimens of BCMA-AB2-3 significantly inhibited tumor growth (p<0.0001, BMCA-AB2-3 vs PBS; two-way ANOVA with Tukey's test on tumor volumes at end points). Both BCMA-AB2-3 regimens induced dramatic and prolonged tumor regressions and eliminated all tumors in about two weeks after initial treatments.

### Example B4: Toxicity of Payloads in SD Rat

One control group of 3 male and 3 female rats was dosed with 0.9% NaCl (“vehicle” or “CNTL”). Three groups of 3 male and 3 female rats each were dosed with a single dose of payload L047-082. Group 1 was dosed with 0.25 mg/kg of payload L047-082. Group 2 was dosed with 0.5 mg/kg of payload L047-082. Group 3 was dosed with 1.0 mg/kg of payload L047-082.

Three groups of 3 male and 3 female rats each were dosed with a single dose of payload L032-060. Group 1 was dosed with 0.25 mg/kg of payload L032-060. Group 2 was dosed with 0.5 mg/kg of payload L032-060. Group 3 was dosed with 1.0 mg/kg of payload L032-060.

Four groups of 3 male and 3 female rats each were dosed with a single dose of payload L044-023C. Group 1 was dosed with 0.5 mg/kg of payload L044-023C. Group 2 was dosed with 1.0 mg/kg of payload L044-023C. Group 3 was dosed with 2.0 mg/kg of payload L044-023C. Group 4 was dosed with 4.0 mg/kg of payload L044-023C.

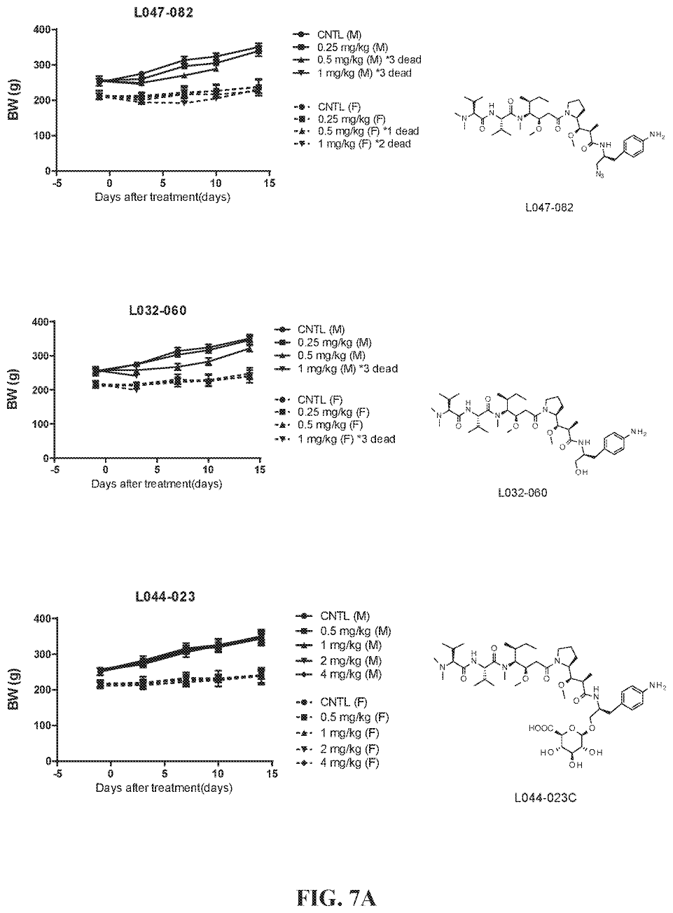

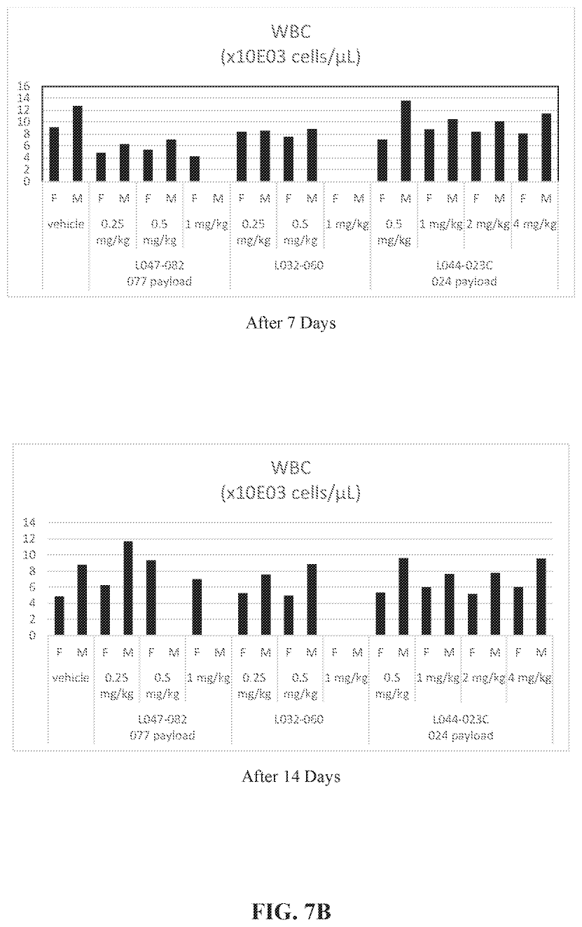

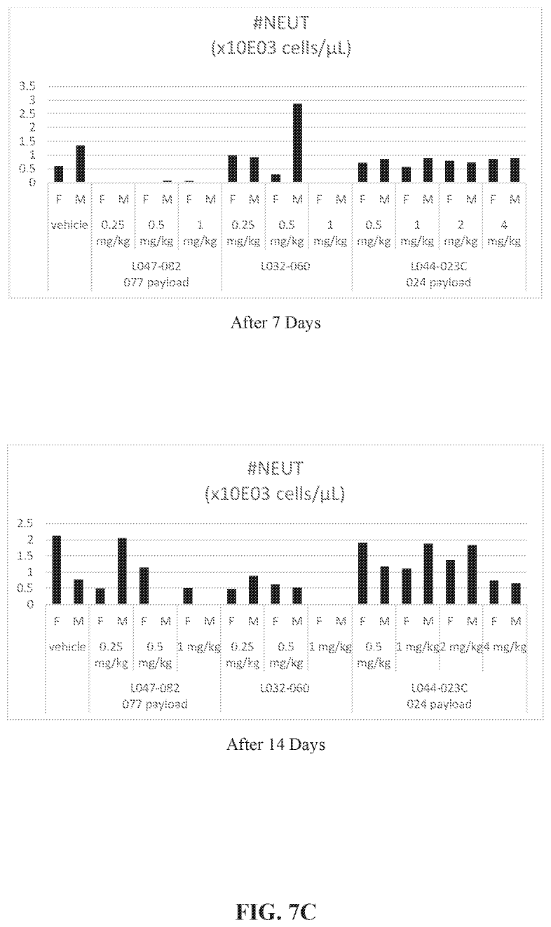

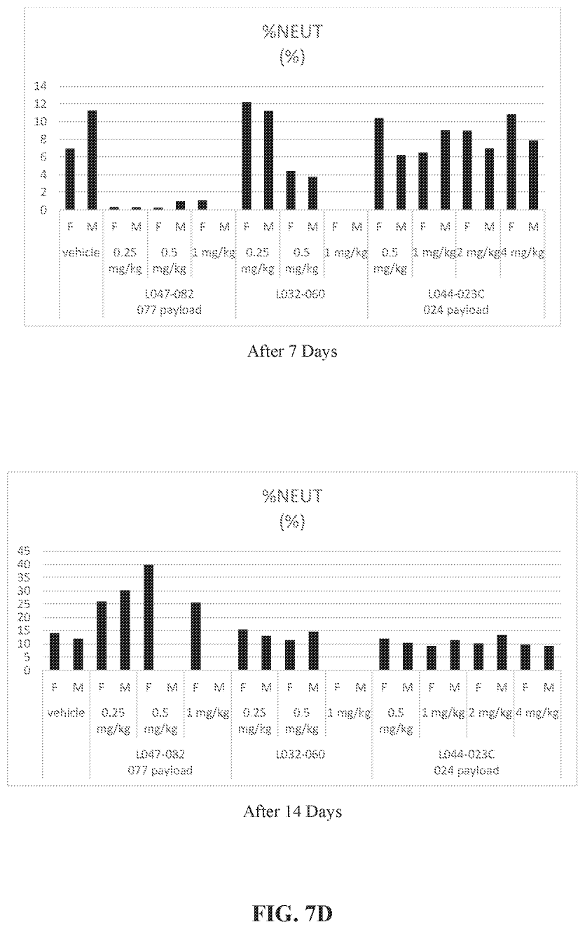

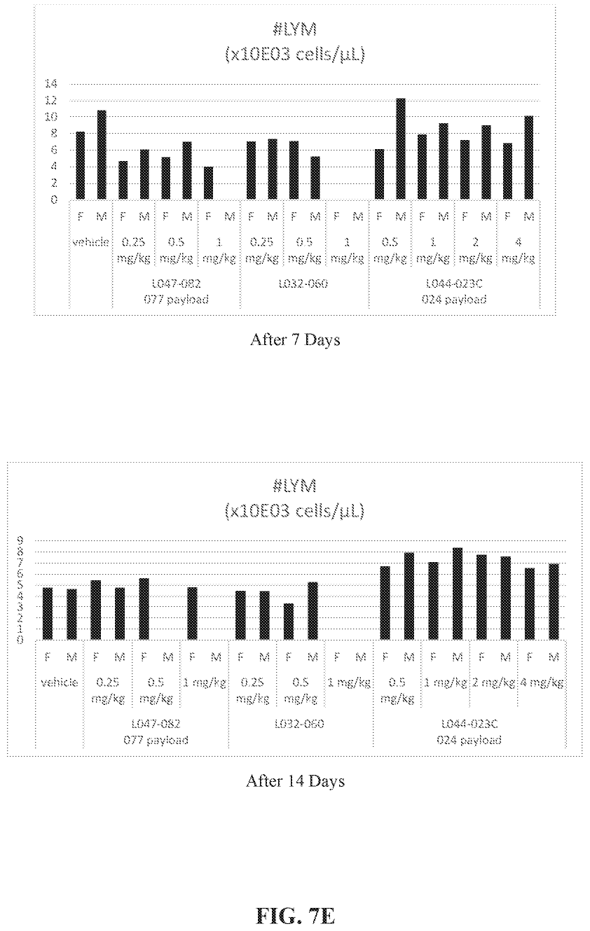

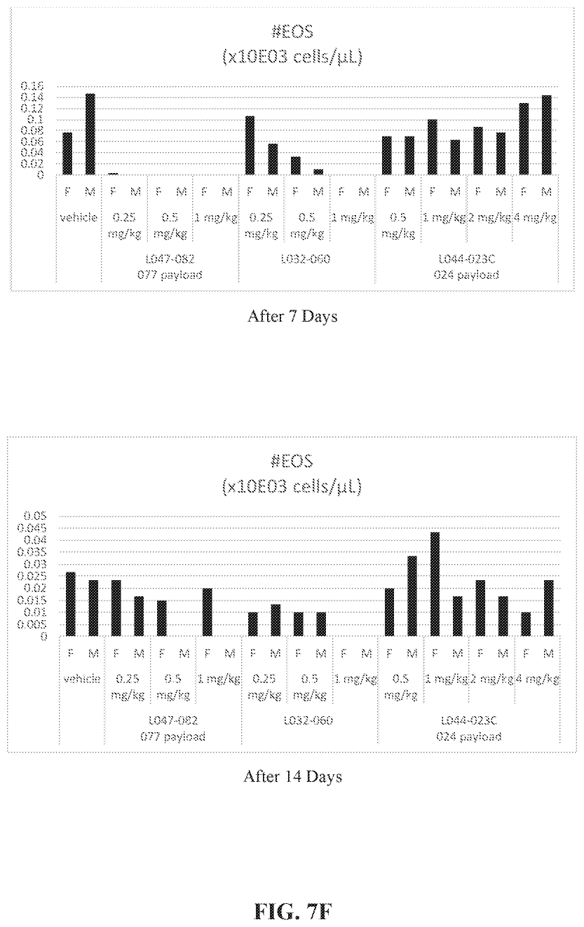

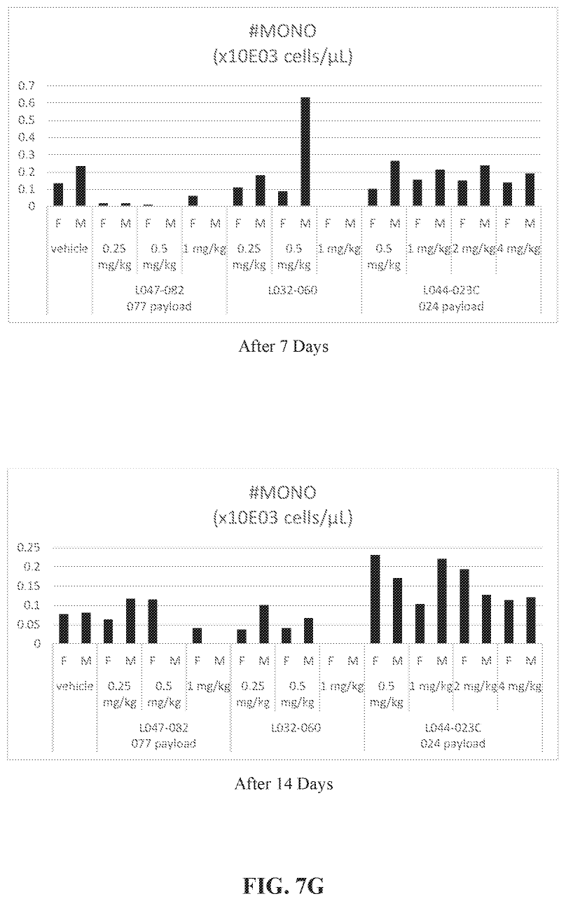

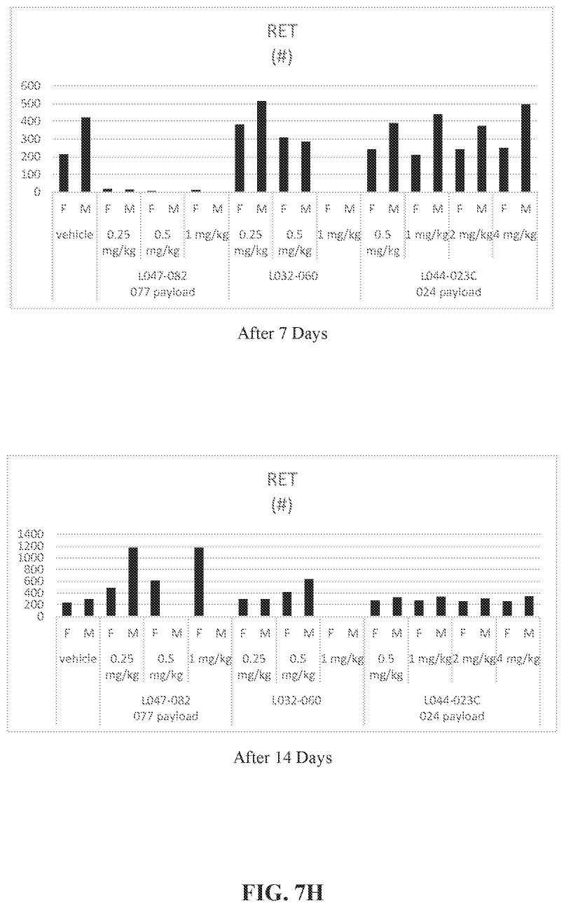

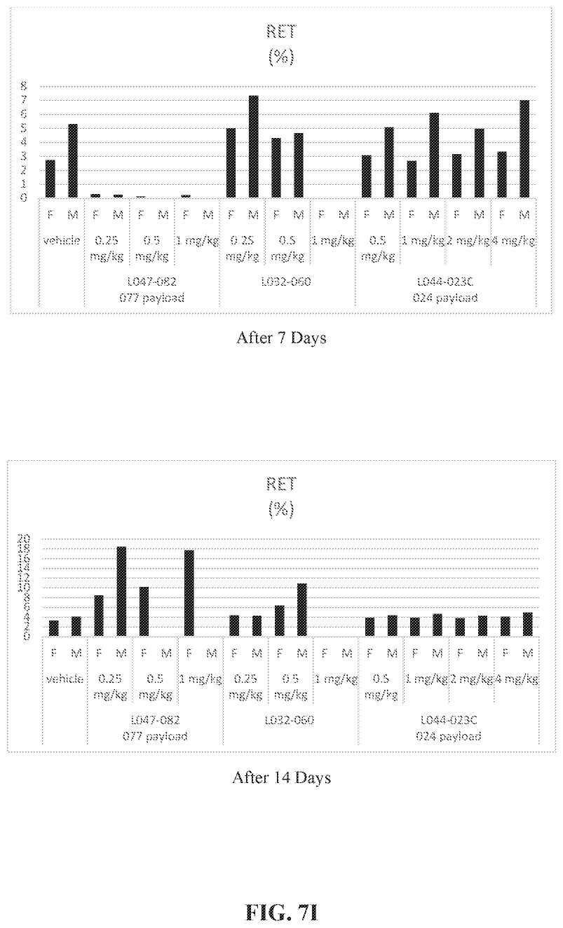

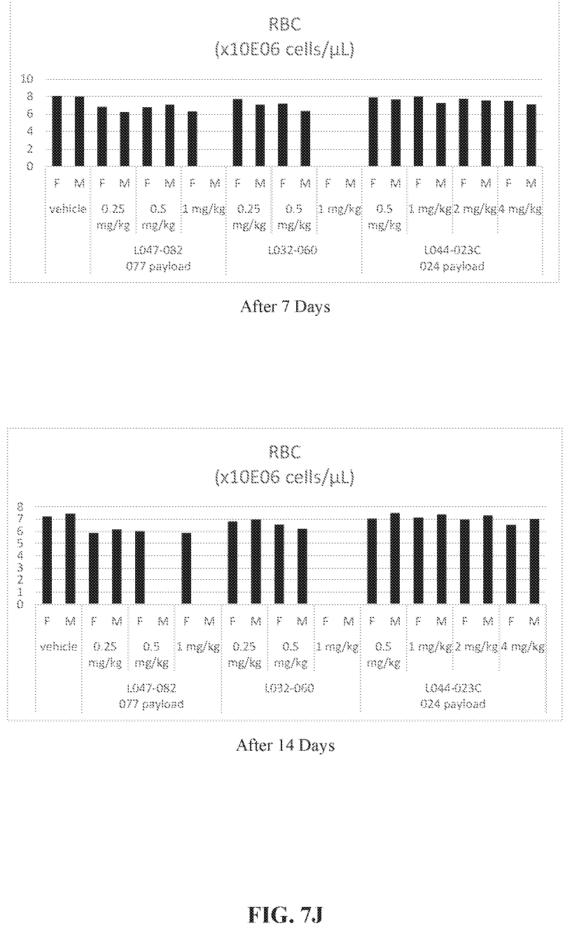

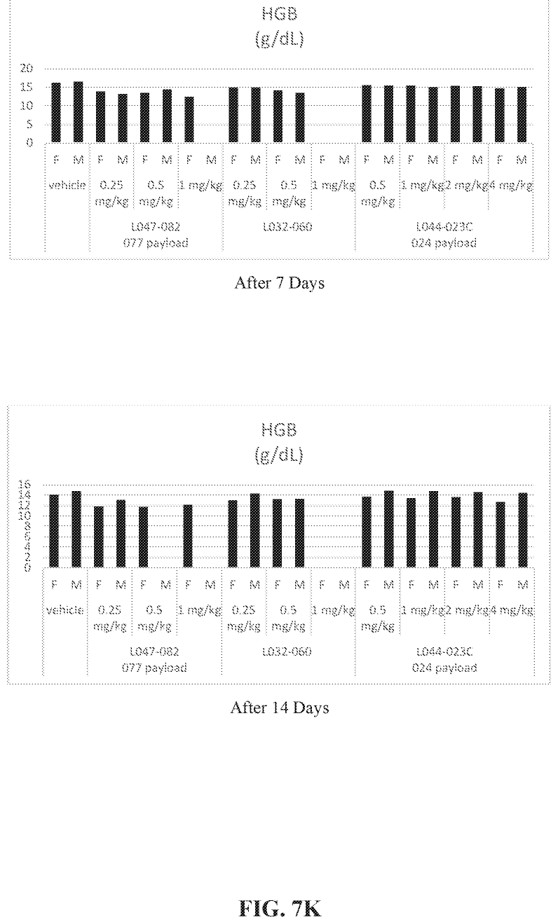

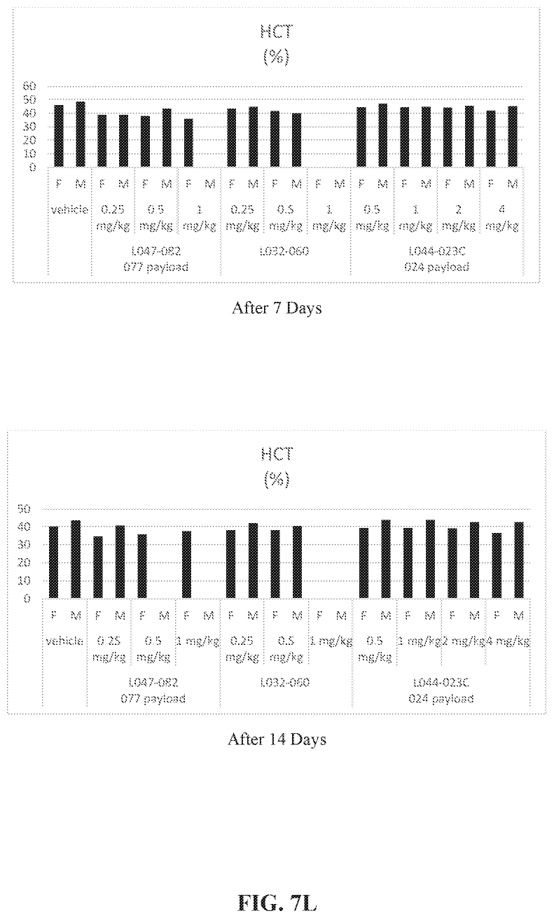

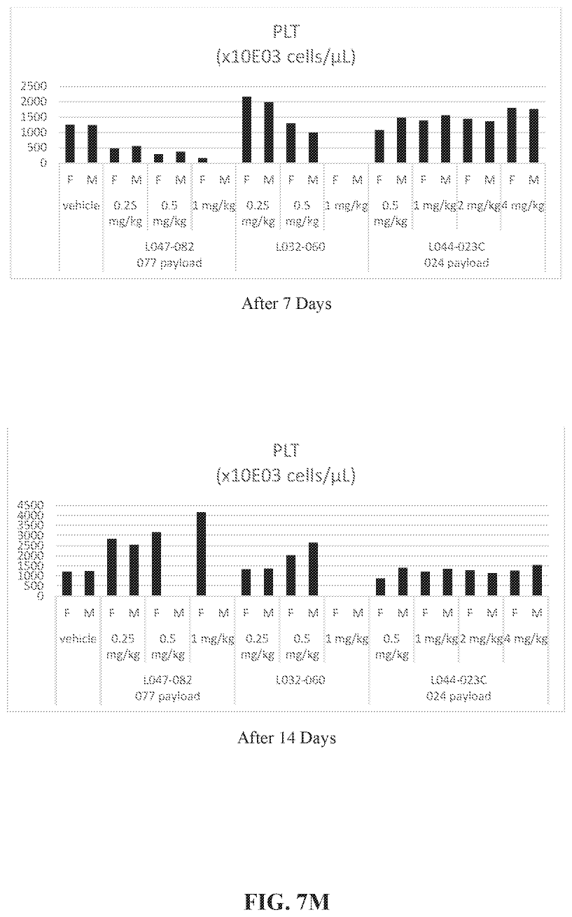

The body weight of all rats was measured once predose and twice weekly after the single payload dose. The body weights of the rats, separated into male (M) and female (F) are shown in FIG. 7A. FIG. 7A also shows the structures of L047-082, L032-060, and L044-023C.

For L047-082 payload 4 out of 6 rats died following treatment with 0.5 mg/kg and 5 out of 6 died following treatment with 1 mg/kg. For L032-060 payload 6 out of 6 died following treatment with 1 mg/kg. For L044-023C payload no rats died even following treatment with 4 mg/kg.

Hematological testing was carried out on day 7 and day 14 after dosing. The following tests were done: white blood cell count, neutrophil count, percent change in neutrophils, lymphocyte count, eosinophil count, monocyte count, reticulocyte count, percent change in reticulocytes, red blood cell count, hemoglobin concentration, percent change in hematocrit, platelet count.

Results of the hematological tests are shown in FIGS. 7B-7M. “(x10E03 cells/μL)” indicates a cell count expressed as thousands of cells per microliter of blood. “(%)” indicates percent change relative to baseline at the indicated time. “(#)” indicates cell count/μL. “(×10E06 cells/μL)” indicates a cell count expressed as millions of cells per microliter of blood. “(g/dL)” indicates grams per deciliter. Definitions of cell type abbreviations are provided above.

L047-082 payload appears to be most toxic with very low neutrophil count, eosinophil count, monocyte count, reticulocyte count, and platelet count at 7 days after dosing. Some recovery is observed 14 days after dosing. L044-023C payload appears to be least toxic even at 4 mg/kg dose.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

What is claimed is:

1. An antibody drug conjugate (ADC) of formula (I): 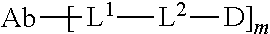
or a pharmaceutically acceptable salt thereof, wherein:
Ab is an anti-BCMA, anti-ROR1, anti-CD25, or anti-Claudine 18 antibody;
m is an integer from 1 to 8;
L¹ is a linker bound to the anti-BCMA, anti-ROR1, anti-CD25, or anti-Claudine 18 antibody;
L² is a bond, —C(O)—, —NH—, Amino Acid Unit, —(CH₂CH₂O)ₙ—, —(CH₂)ₙ—, -(4-aminobenzyloxycarbonyl)-, 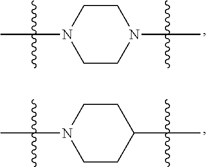
—(C(O)CH₂CH₂NH)—, or combinations thereof; wherein n is an integer from 1 to 24; and
D is a drug moiety.

2. The ADC of claim 1, wherein Ab is an anti-BCMA antibody.

3. The ADC of claim 1 or 2, wherein L¹ is a linker bound to one or two sulfur or nitrogen atoms of the anti-BCMA antibody.

4. The ADC of any one of claims 1-3, wherein L¹ is:

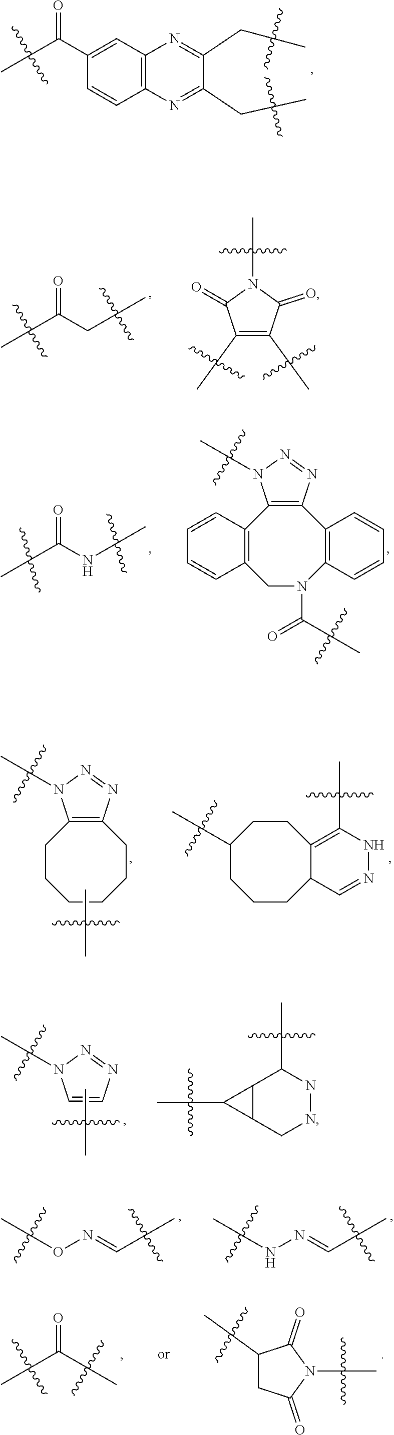

5. The ADC of any one of claims 1-4, wherein m is 1, 2, 3, 4, 5, 6, 7, or 8.

6. The ADC of claim 5, wherein m is from 2 to 8.

7. The ADC of any one of claims 1-6, wherein L² is a bond, —C(O)—, —NH—, Val, Phe, Lys, -(4-aminobenzyloxycarbonyl)-, 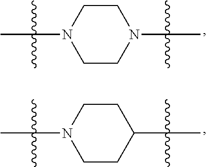
Gly, Ser, Thr, Ala, β-Ala, citrulline (Cit), —(CH₂)ₙ—, —(CH₂CH₂O)ₙ—, or combinations thereof.

8. The ADC of claim 7, wherein L² is a bond, —C(O)—, —NH—, Val, Phe, Lys, -(4-aminobenzyloxycarbonyl)-, 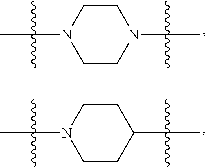
—(CH)ₙ—, —(CH₂CH₂O)ₙ—, or combinations thereof.

9. The ADC of claim 7, wherein L² is a bond, —C(O)—, —NH—, Gly, Ser, Thr, Ala, β-Ala, Cit, 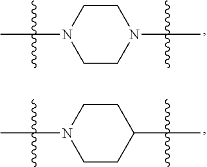
—(CH₂)ₙ—, —(CH₂CH₂O)ₙ—, or combinations thereof.

10. The ADC of claim 7, wherein L² is a bond, 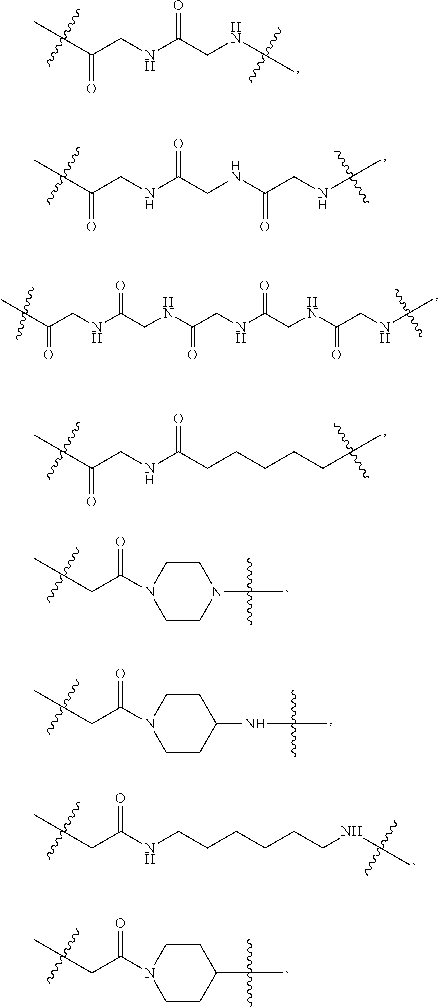
or —C(O)—(CH₂)₅—.

11. The ADC of claim 10, wherein L² is

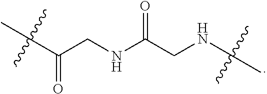 [SMILES: *NCC(=O)NCC(*)=O]

12. The ADC of claim 10, wherein L² is

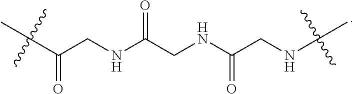 [SMILES: *NCC(=O)NCC(=O)NCC(*)=O]

13. The ADC of claim 10, wherein L² is

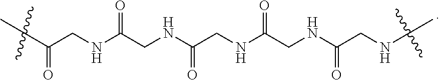 [SMILES: *NCC(=O)NCC(=O)NCC(=O)NCC(=O)NCC(*)=O]

14. The ADC of claim 10, wherein L² is —C(O)—(CH₂)₅—.

15. The ADC of claim 10, wherein L² is

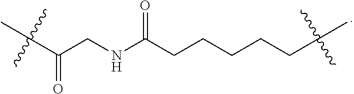 [SMILES: *CCCCCC(=O)NCC(*)=O]

16. The ADC of claim 10, wherein L² is

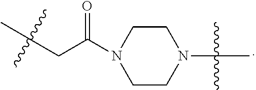 [SMILES: *CC(=O)N1CCN(*)CC1]

17. The ADC of claim 10, wherein L² is

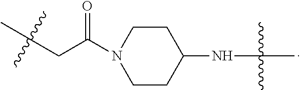 [SMILES: *CC(=O)N1CCC(N*)CC1]

18. The ADC of claim 10, wherein L² is

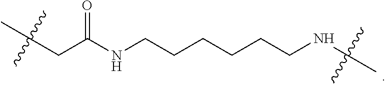 [SMILES: *CC(=O)NCCCCCCN*]

19. The ADC of claim 10, wherein L² is

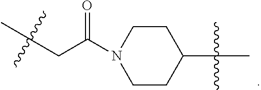 [SMILES: *CC(=O)N1CCC(*)CC1]

20. The ADC of any one of claims 1-19, wherein D is 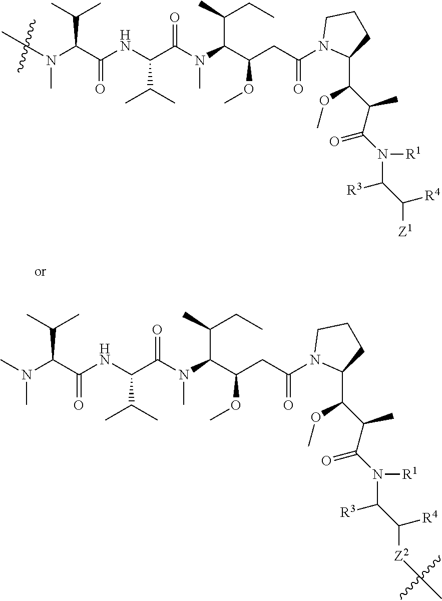
wherein:
R¹ is H or —C₁-C₈ alkyl;
R³ is H, halogen, —CCl₃, —CBr₃, —CF₃, —Cl₃, —CHCI₂, —CHBr₂, —CHF₂, —CHI₂, —CH₂Cl, —CH₂Br, —CH₂F, —CH₂I, —CN, —OR^{3A}, —NR^{3A}R^{3B}, —(CH₂)ᵥOR⁶, 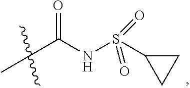 [SMILES: *C(=O)NS(=O)(=O)C1CC1]
substituted or unsubstituted alkyl, or substituted or unsubstituted heteroalkyl;
R⁴ is H, halogen, —OR^{4A}, —NR^{4A}R^{4B}, substituted or unsubstituted alkyl, or substituted or unsubstituted heteroalkyl:
Z¹ is a substituted or unsubstituted aryl, substituted or unsubstituted heteroaryl, substituted or unsubstituted cycloalkyl, or substituted or unsubstituted heterocycloalkyl;
Z² is a substituted or unsubstituted arylene, substituted or unsubstituted heteroarylene, substituted or unsubstituted cycloalkylene, or substituted or unsubstituted heterocycloalkylene;
R⁶ is H, substituted or unsubstituted alkyl, substituted or unsubstituted cycloalkyl, substituted or unsubstituted heterocycloalkyl, substituted or unsubstituted aryl, substituted or unsubstituted heteroaryl, —CO(CH₂CH₂O)_{w}CH₂CH₂Y, —CONH(CH₂CH₂O)_{w}CH₂CH₂Y, 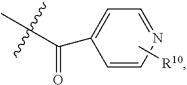
a Charged Group, or a saccharide derivative, wherein
v is an integer from 1 to 24; w is an integer from 1 to 24; Y is —NH₂, —OH, —COOH, or —OCH₃;
R¹⁰ is —OH, —OCH₃ or —COOH; and
each R^{3A}, R^{3B}, R^{4A}, and R^{4B} is independently H or substituted or unsubstituted alkyl.

21. The ADC of claim 20, wherein R¹ is H.

22. The ADC of claim 20 or 21, wherein R³ is H, —OR^{3A}, —(CH₂)ᵥR⁶, 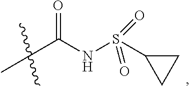 [SMILES: *C(=O)NS(=O)(=O)C1CC1]
substituted or unsubstituted alkyl, or substituted or unsubstituted heteroalkyl.

23. The ADC of claim 22, wherein R³ is H, —OR^{3A}, —(CH₂)ᵥOR⁶, 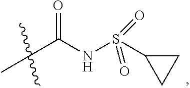 [SMILES: *C(=O)NS(=O)(=O)C1CC1]
unsubstituted C₁-C₆ alkyl, or substituted C₁-C₆ alkyl.

24. The ADC of claim 22, wherein R³ is H, methyl, ethyl, propyl, butyl, —CH₂OH, —CH₂CH₂OH, —CH₂N₃, —CH₂CH₂N₃, —CH₂OCH₃, —CH₂OCH₂CH₃, —CH₂CH₂OCH₃, —CH₂CH₂OCH₂CH₃,

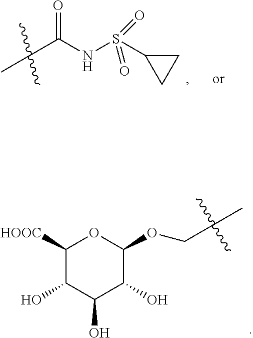 [SMILES: *C(=O)NS(=O)(=O)C1CC1 ; *CO[C@@H]1O[C@H](C(=O)O)[C@@H](O)[C@H](O)[C@H]1O]

25. The ADC of claim 24, wherein R³ is methyl, —CH₂OH, —CH₂N₃,

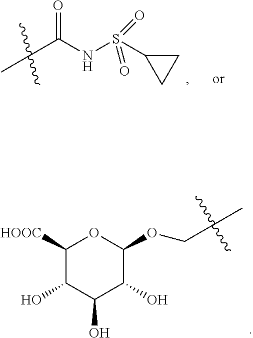 [SMILES: *C(=O)NS(=O)(=O)C1CC1 ; *CO[C@@H]1O[C@H](C(=O)O)[C@@H](O)[C@H](O)[C@H]1O]

26. The ADC of any one of claims 20-25, wherein R⁴ is H, —OR^{4A}, substituted or unsubstituted alkyl, or substituted or unsubstituted heteroalkyl.

27. The ADC of claim 26, wherein R⁴ is H, —OH, methyl, ethyl, propyl or butyl.

28. The ADC of claim 27, wherein R⁴ is H or —OH.

29. The ADC of any one of claims 20-28, wherein Z¹ is a substituted or an unsubstituted aryl.

30. The ADC of any one of claims 20-28, wherein Z² is an unsubstituted arylene.

31. The ADC of claim 29, wherein Z¹ is 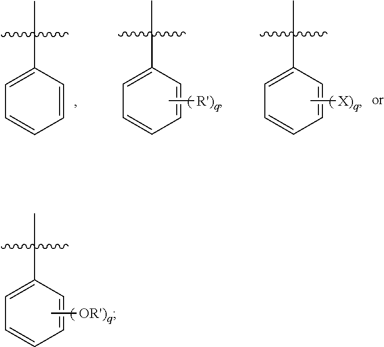
wherein
each X is independently Cl, Br, I, or F;
each R¹ is independently —CH₃, —CH₂CH₃ or —CH₂CH₂CH₃; and
q is an integer from 1 to 5.

32. The ADC of claim 31, wherein Z¹ is

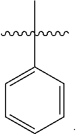 [SMILES: *c1ccccc1]

33. The ADC of claim 30, wherein Z² is 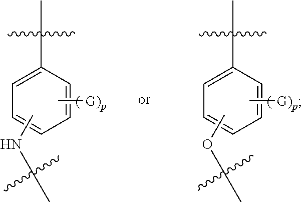
wherein
each G is independently Cl, Br, I, F, —CH₃, —CH₂CH₃, —CH₂CH₂CH₃, —OCH₃, —OCH₂CH₃, —OH, or —NH₂; and p is an integer from 0-4.

34. The ADC of claim 33, wherein Z² is

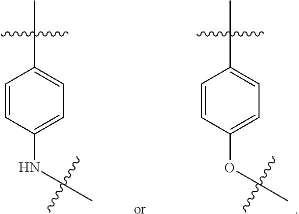 [SMILES: *Nc1ccc(*)cc1 ; *Oc1ccc(*)cc1 ; "or" ; "."]

35. The ADC of any one of claims 1-34, wherein D is:

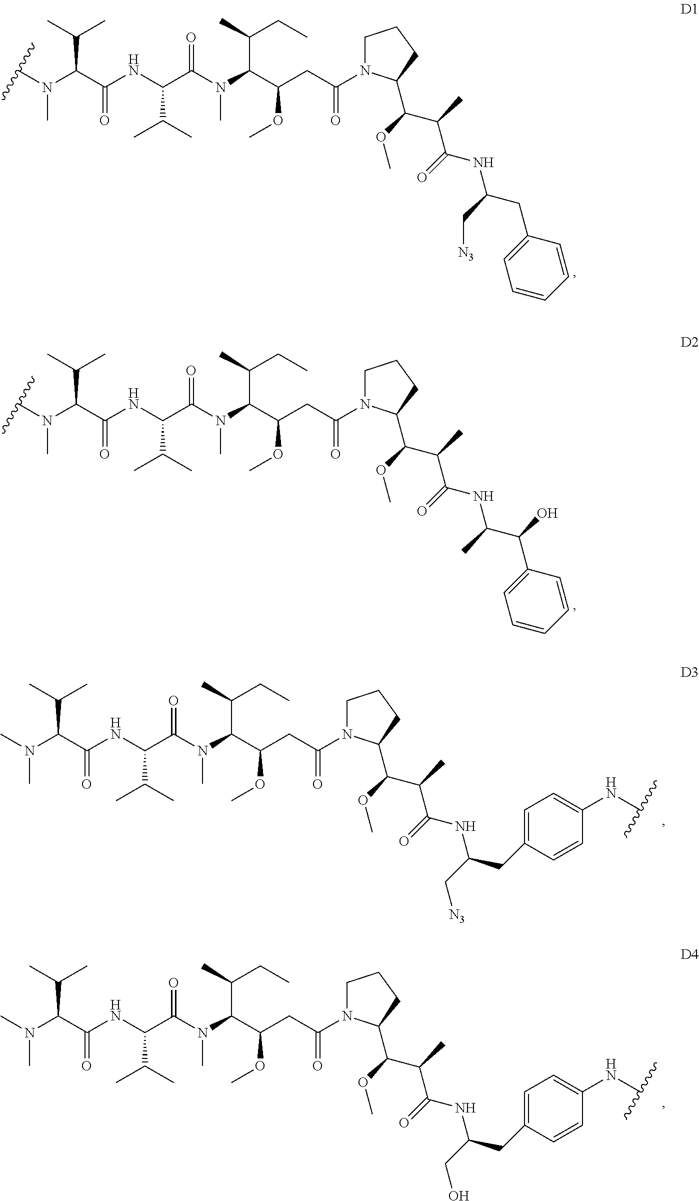

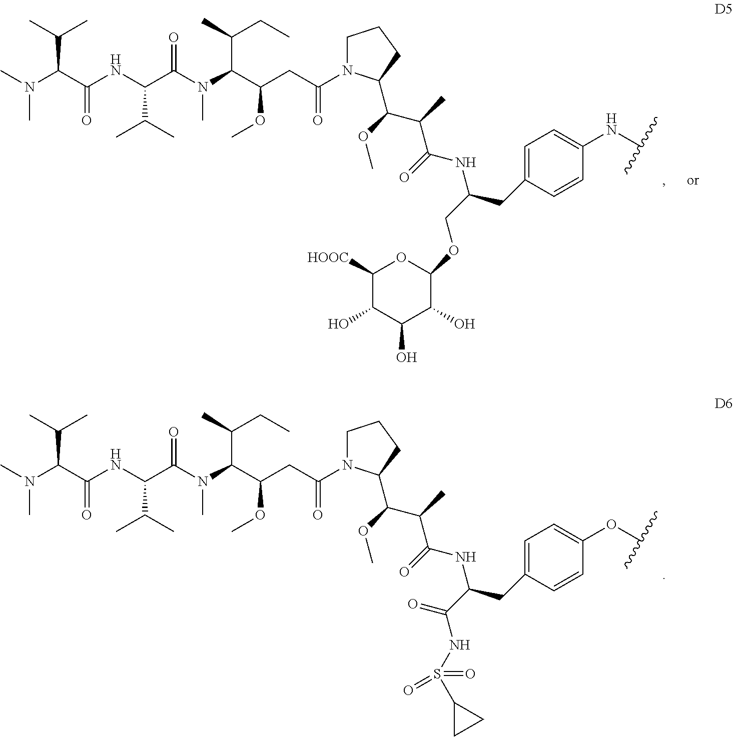 [SMILES: *Nc1ccc(C[C@@H](CO[C@@H]2O[C@H](C(=O)O)[C@@H](O)[C@H](O)[C@H]2O)NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)cc1 ; *Oc1ccc(C[C@H](NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)C(=O)NS(=O)(=O)C2CC2)cc1]

36. The ADC of claim 35, wherein D is

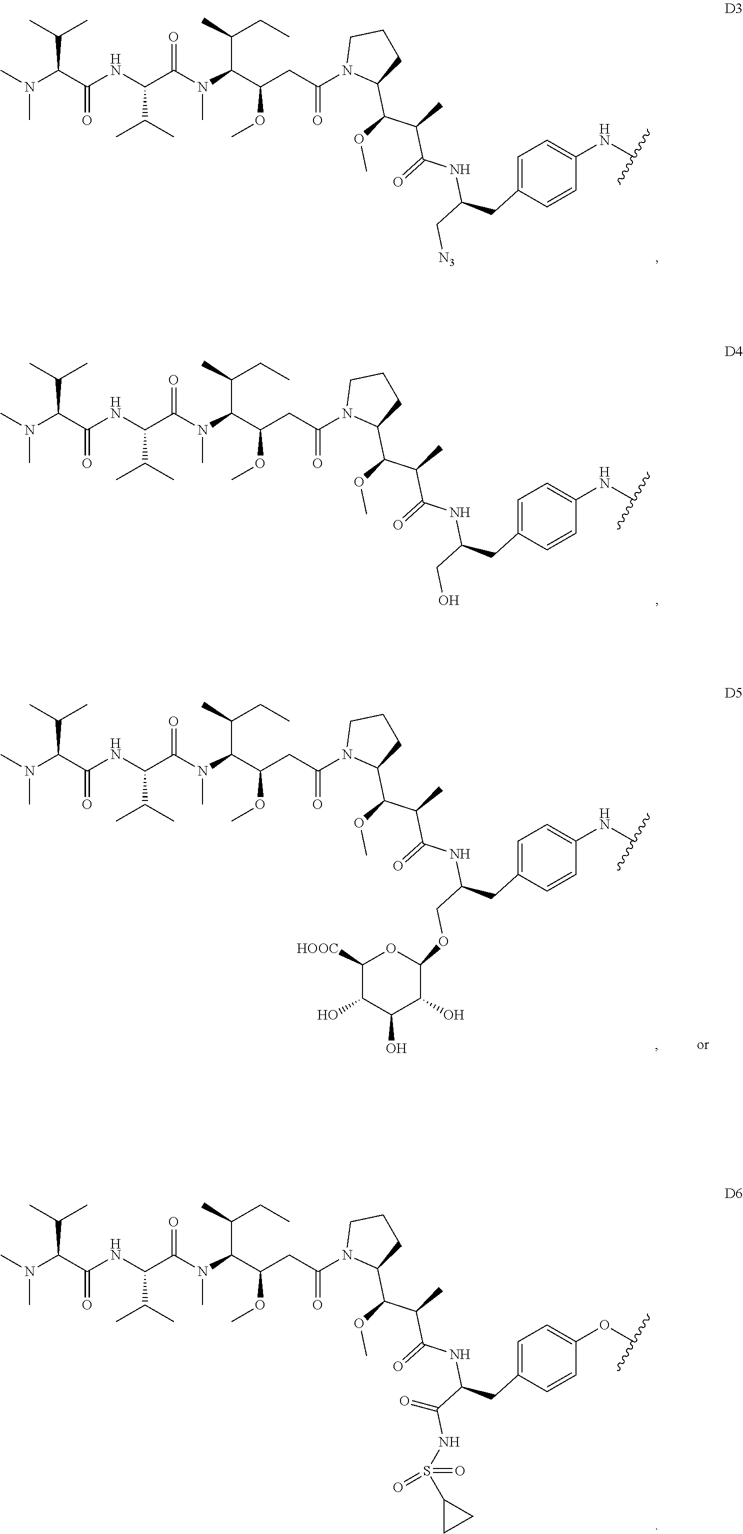

37. The AM of claim 36, wherein D is

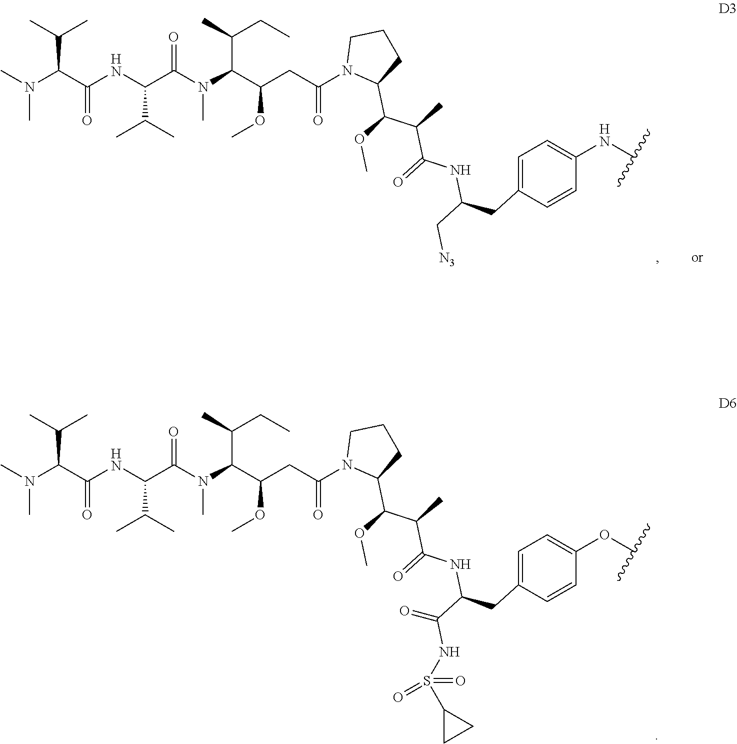 [SMILES: *Nc1ccc(C[C@@H](CN=[N+]=[N-])NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)cc1 ; *Oc1ccc(C[C@H](NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)C(=O)NS(=O)(=O)C2CC2)cc1]

38. The ADC of any one of claims 1-37, wherein the ADC is: 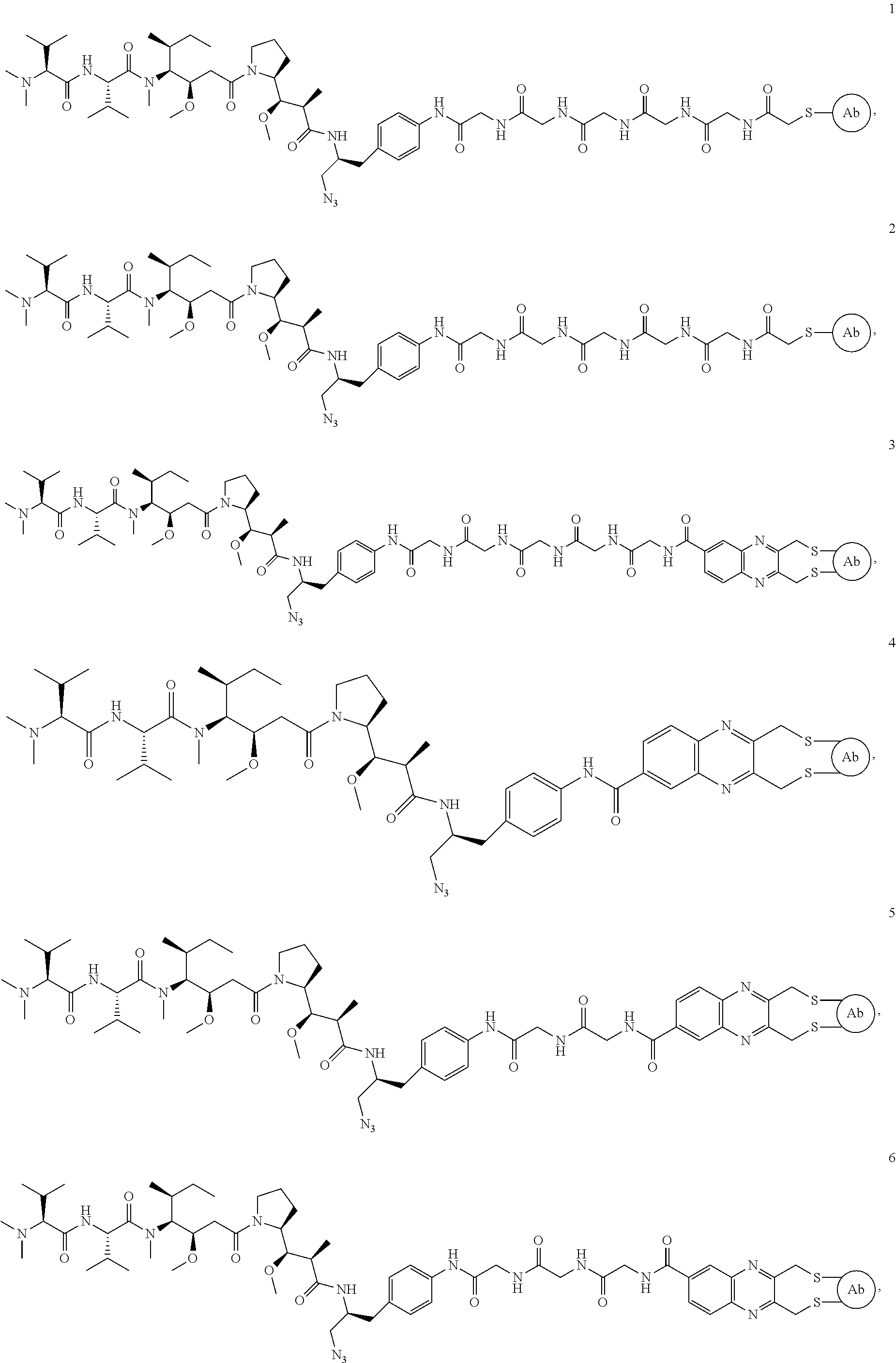 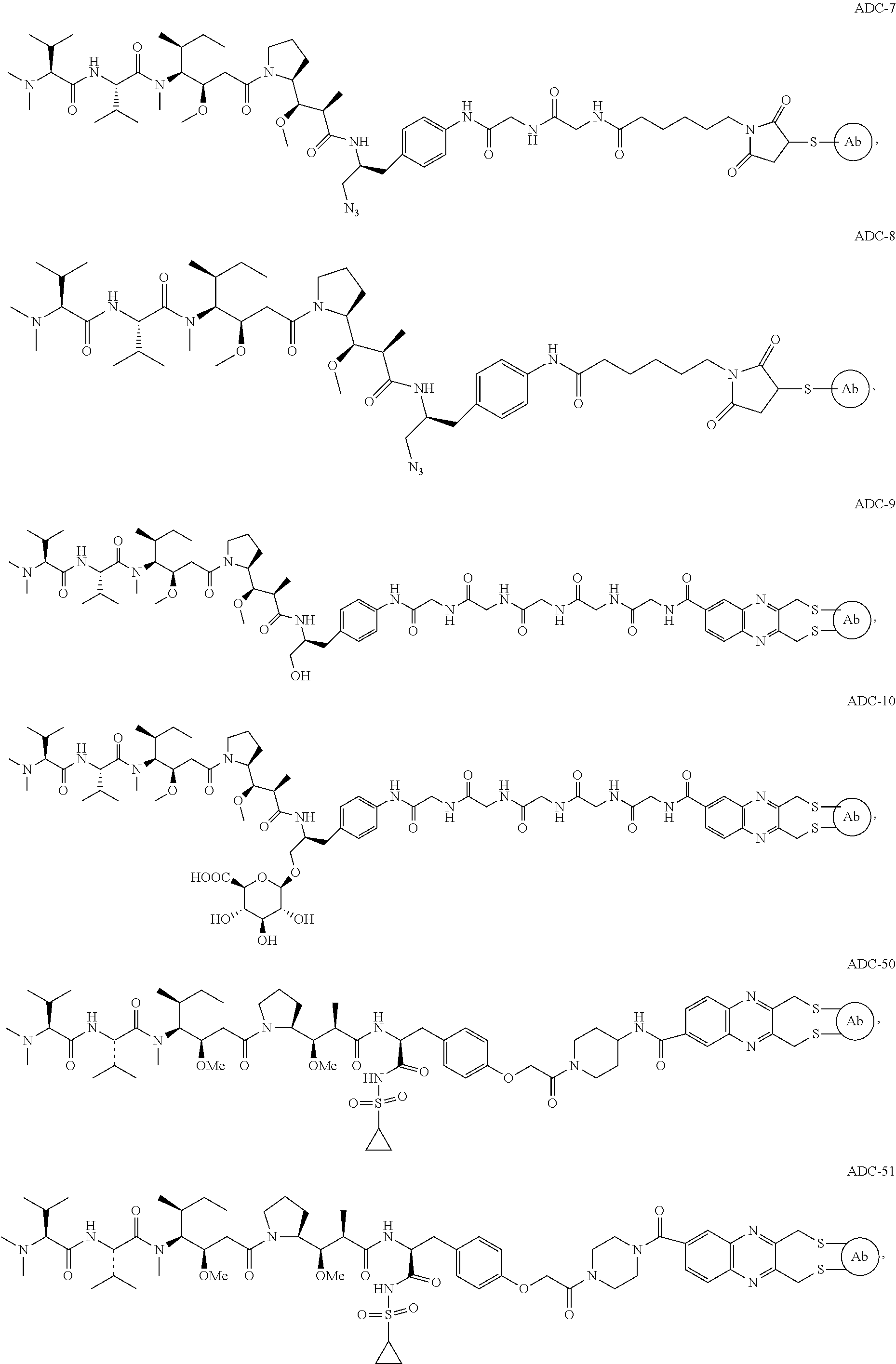 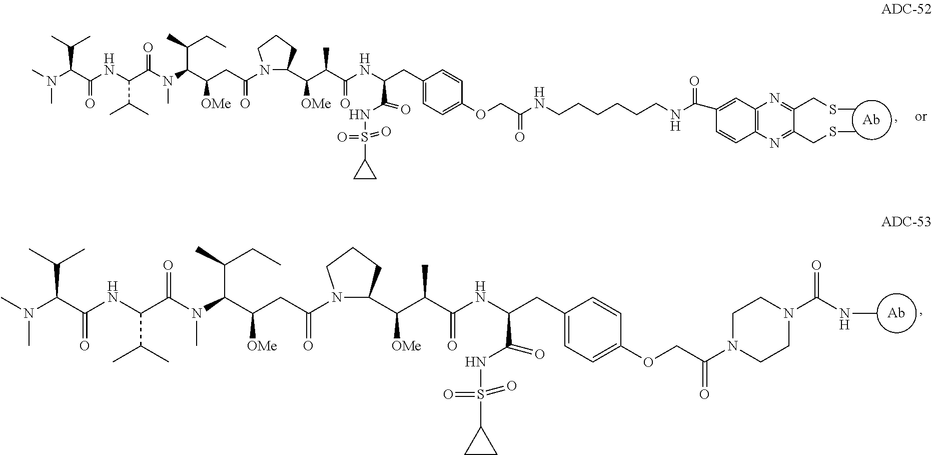
or a pharmaceutically acceptable salt thereof.

39. The ADC of any one of claims 1-38, wherein the anti-BCMA antibody comprises a VL CDR1 comprising the sequence of SEQ ID NO: 1, a VL CDR2 comprising the sequence of SEQ ID NO: 2, a VL CDR3 comprising the sequence of SEQ ID NO: 3, a VH CDR1 comprising the sequence of SEQ ID NO: 4, a VH CDR2 comprising the sequence of SEQ ID NO: 5, and a VH CDR3 comprising the sequence of SEQ ID NO: 6.

40. The ADC of any one of claims 1-39, wherein the anti-BCMA antibody comprises a VL having a sequence with at least 95%, 96%, 97%, 98%, or 99% identity to SEQ ID NO: 7.

41. The ADC of any one of claims 1-40, wherein the anti-BCMA antibody comprises a VH having a sequence with at least 95%, 96%, 97%, 98%, or 99% identity to SEQ ID NO: 8.

42. The ADC of any one of claims 1-41, wherein the anti-BCMA antibody comprises a VL having the sequence of SEQ ID NO: 7.

43. The ADC of any one of claims 1-42, wherein the anti-BCMA antibody comprises a VH having the sequence of SEQ ID NO: 8.

44. The ADC of any one of claims 1-43, wherein the anti-BCMA antibody is an IgG antibody, optionally wherein the anti-BCMA antibody is an IgG1 antibody.

45. The ADC of any one of claims 1-44, wherein the anti-BCMA antibody binds a human BCMA, optionally wherein the human BCMA has the amino acid sequence of SEQ ID NO: 16.

46. The ADC of any one of claims 1-45, for use in therapy.

47. The ADC of claim 46, for use in treating a BCMA-expressing cancer.

48. A method of treating a BCMA-expressing cancer in a subject, comprising administering the ADC of any one of claims 1-45 to a subject in need thereof.

49. Use of the ADC of any one of claims 1-45 for the manufacture of a medicament.

50. Use of the ADC of any one of claims 1-45 for the manufacture of a medicament for treating a BCMA-expressing cancer.

51. The ADC for use or method of any one of claims 47, 48, or 50, wherein the BCMA-expressing cancer is multiple myeloma.

52. A method of preparing the ADC of any one of claims 1-47, comprising reacting an anti-BCMA, anti-ROR1, anti-CD25, or anti-Claudine 18 antibody with a molecule of formula (P-I):
B-L²-D
or a pharmaceutically acceptable salt thereof, wherein:
B is a reactive moiety capable of forming a bond with the anti-BCMA, anti-ROR1, anti-CD25, or anti-Claudine 18 antibody;
L² is a bond, —C(O)—, —NH—, Amino Acid Unit, —(CH₂CH₂O)ₙ—, —(CH₂)ₙ—, -(4-aminobenzyloxycarbonyl)-, 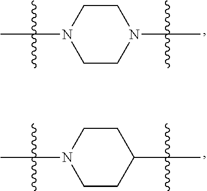 [SMILES: *N1CCN(*)CC1 ; *C1CCN(*)CC1]
—(C(O)CH₂CH₂NH)— or combinations thereof, where n is an integer from 1 to 24; and
D is a drug moiety.

53. The method of claim 52, wherein the antibody is modified with an aldehyde, azide, alkyne, tetrazine, hydrazine, alkoxyamine, trans-cyclooctene or cyclopropene.

54. The method of claim 52 or 53, wherein the antibody is an anti-BCMA antibody.

55. The method of claim 54, wherein B is a reactive moiety capable of forming a bond with one or two thiol or amine groups of the anti-BCMA antibody, or with the modified anti-BCMA antibody.

56. The method of claim 55, wherein B is:

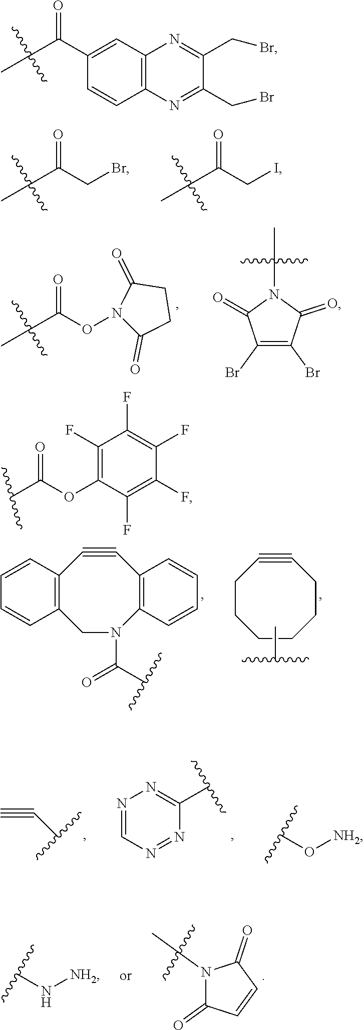

57. The method of claim 52, wherein L² is a bond, —C(O)—, —NH—, Val, Phe, Lys, -(4-aminobenzyloxycarbonyl)-, Gly, Ser, Thr, Ala, β-Ala, citrulline (Cit), 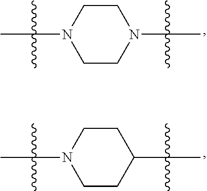 [SMILES: *N1CCN(*)CC1 ; *C1CCN(*)CC1]
—(CH₂)ₙ, —(CH₂CH₂O)ₙ—, or combinations thereof.

58. The method of claim 52, wherein L² is a bond, —C(O)—, —NH—, Val, Phe, Lys, -(4-aminobenzyloxycarbonyl)-, 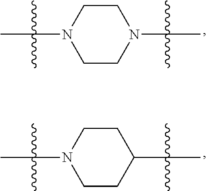 [SMILES: *N1CCN(*)CC1 ; *C1CCN(*)CC1]
—(CH₂)ₙ—, —(CH₂CH₂O)ₙ—, or combinations thereof.

59. The method of claim 52, wherein L² is a bond, —C(O)—, —NH—, Gly, Ser, Thr, Ala, β-Ala, Cit, 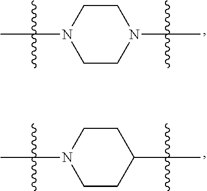 [SMILES: *N1CCN(*)CC1 ; *C1CCN(*)CC1]
—(CH₂)ₙ—, —(CH₂CH₂O)ₙ—, or combinations thereof.

60. The method of claim 52, wherein L² is a bond, 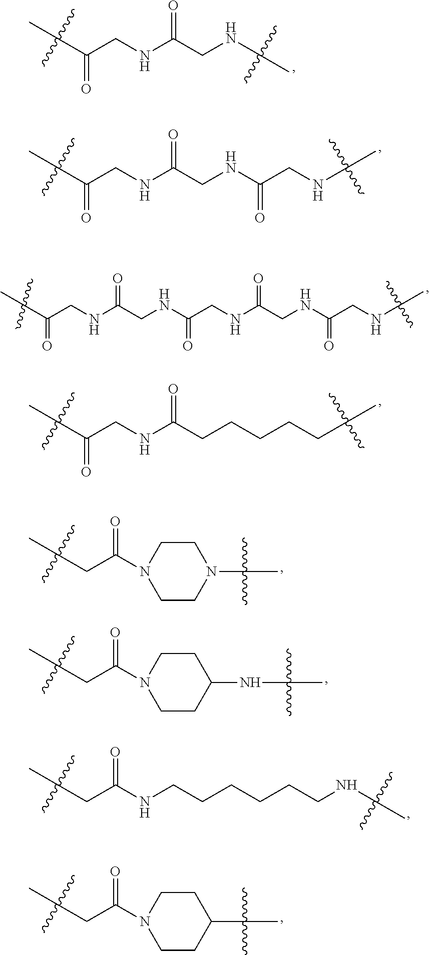
or —C(O)—(CH₂)₅—.

61. The method of claim 60, wherein L² is

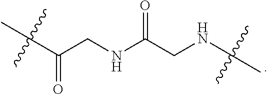 [SMILES: *NCC(=O)NCC(*)=O]

62. The method of claim 60, wherein L² is

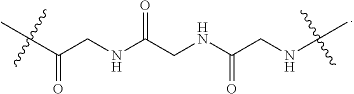 [SMILES: *NCC(=O)NCC(=O)NCC(*)=O]

63. The method of claim 60, wherein L² is

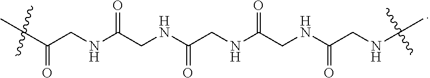 [SMILES: *NCC(=O)NCC(=O)NCC(=O)NCC(=O)NCC(*)=O]

64. The method of claim 60, wherein L² is —C(O)—(CH₂)₅—.

65. The method of claim 60, wherein L² is

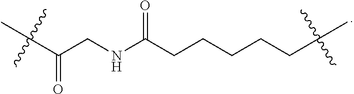 [SMILES: *CCCCCC(=O)NCC(*)=O]

66. The method of claim 60, wherein L² is

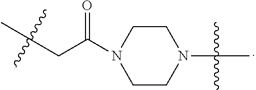 [SMILES: *CC(=O)N1CCN(*)CC1]

67. The method of claim 60, wherein L² is

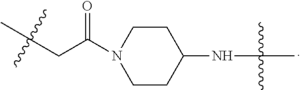 [SMILES: *CC(=O)N1CCC(N*)CC1]

68. The method of claim 60, wherein L² is H

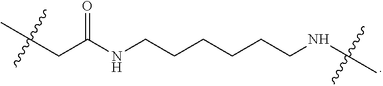 [SMILES: *CC(=O)NCCCCCCN*]

69. The method of claim 60, wherein L² is

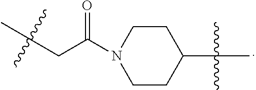 [SMILES: *CC(=O)N1CCC(*)CC1]

70. The method of claim 52, wherein D is 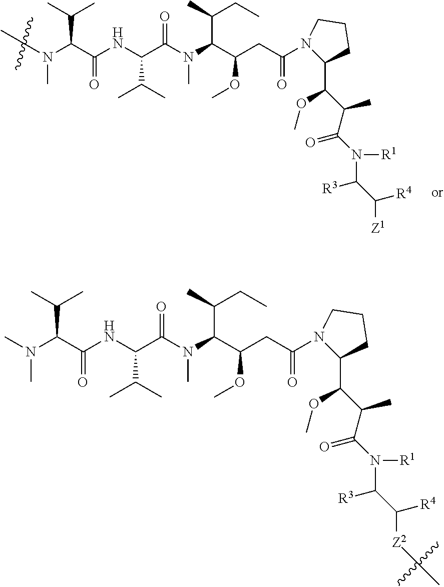
wherein:
R¹ is H or —C₁-C₈ alkyl;
R³ is H, halogen, -CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OR^{3A}, —NR^{3A}R^{3B}, —(CH₂)ᵥR⁶, 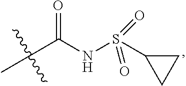 [SMILES: *C(=O)NS(=O)(=O)C1CC1]
substituted or unsubstituted alkyl, or substituted or unsubstituted heteroalkyl;
R⁴ is H, halogen, —OR^{4A}, —NR^{4A}R^{4B}, substituted or unsubstituted alkyl, or substituted or unsubstituted heteroalkyl;
Z¹ is a substituted or unsubstituted aryl, substituted or unsubstituted heteroaryl, substituted or unsubstituted cycloalkyl, or substituted or unsubstituted heterocycloalkyl;
Z² is a substituted or unsubstituted arylene, substituted or unsubstituted heteroarylene, substituted or unsubstituted cycloalkylene, or substituted or unsubstituted heterocycloalkylene;
R⁶ is H, substituted or unsubstituted alkyl, substituted or unsubstituted cycloalkyl, substituted or unsubstituted heterocycloalkyl, substituted or unsubstituted aryl, or substituted or unsubstituted heteroaryl, —CO(CH₂CH₂O)_{w}CH₂CH₂Y, —CONH(CH₂CH₂O)CH₂CH₂Y, 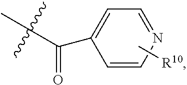
a Charged Group, or a saccharide derivative, wherein
v is an integer from 1 to 24; w is an integer from 1 to 24; Y is —NH₂, —OH, —COOH, or —OCH₃;
R¹⁰ is —OH, —OCH₃ or —COOH; and
each R^{3A}, R^{3B}, R^{4A}, and R^{4B} is independently H or substituted or unsubstituted alkyl.

71. The method of claim 70, wherein R¹ is H.

72. The method of claim 70, wherein R³ is H, —OR^{3A}, —(CH₂) OR⁶, 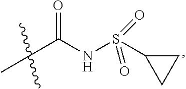 [SMILES: *C(=O)NS(=O)(=O)C1CC1]
substituted or unsubstituted alkyl, or substituted or unsubstituted heteroalkyl.

73. The method of claim 72, wherein R³ is H, —OR^{3A}, —(CH₂)OR⁶, 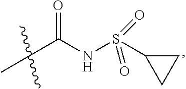 [SMILES: *C(=O)NS(=O)(=O)C1CC1]
unsubstituted C₁-C₆ alkyl, or substituted C₁-C₆ alkyl.

74. The method of claim 73, wherein R³ is H, methyl, ethyl, propyl, butyl, —CH₂OH, —CH₂CH₂OH, —CH₂N₃, —CH₂CH₂N₃, —CH₂OCH₃, —CH₂OCH₂CH₃, —CH₂CH₂OCH₃, —CH₂CH₂OCH₂CH₃,

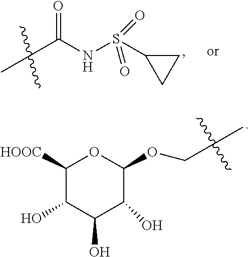 [SMILES: *C(=O)NS(=O)(=O)C1CC1 ; *CO[C@@H]1O[C@H](C(=O)O)[C@@H](O)[C@H](O)[C@H]1O]

75. The method of claim 74, wherein R³ is methyl, —CH₂OH, —CH₂N₃,

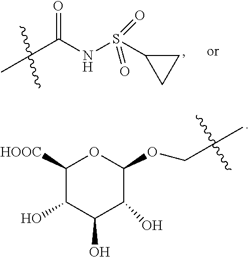 [SMILES: *C(=O)NS(=O)(=O)C1CC1 ; *CO[C@@H]1O[C@H](C(=O)O)[C@@H](O)[C@H](O)[C@H]1O]

76. The method of claim 70, wherein R⁴ is H, —OR^{4A}, substituted or unsubstituted alkyl, or substituted or unsubstituted heteroalkyl.

77. The method of claim 76, wherein R⁴ is H, —OH, methyl, ethyl, propyl or butyl.

78. The method of claim 77, wherein R⁴ is H or —OH.

79. The method of claim 70, wherein Z¹ is a substituted or an unsubstituted aryl.

80. The method of claim 70, wherein Z² is an unsubstituted arylene.

81. The method of claim 79, wherein Z¹ is 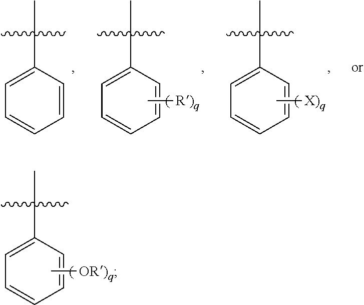
wherein
each X is independently Cl, Br, I, or F;
each R′ is independently —CH₃, —CH₂CH₃ or —CH₂CH₂CH₃; and
q is an integer from 1 to 5.

82. The method of claim 81, wherein Z¹ is

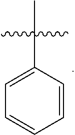 [SMILES: *c1ccccc1]

83. The method of claim 80, wherein Z² is 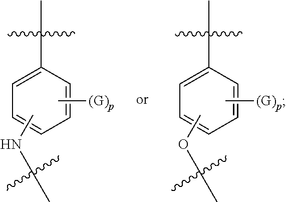
wherein
each G is independently Cl, Br, I, F, —CH₃, —CH₂CH₃, —CH₂CH₂CH₃, —OCH₃, —OCH₂CH₃, —OH, or —NH₂; and p is an integer from 0-4.

84. The method of claim 83, wherein Z² is

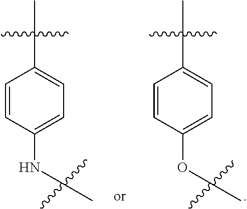 [SMILES: *Nc1ccc(*)cc1 ; *Oc1ccc(*)cc1]

85. The method of any one of claims 52-84, wherein D is:

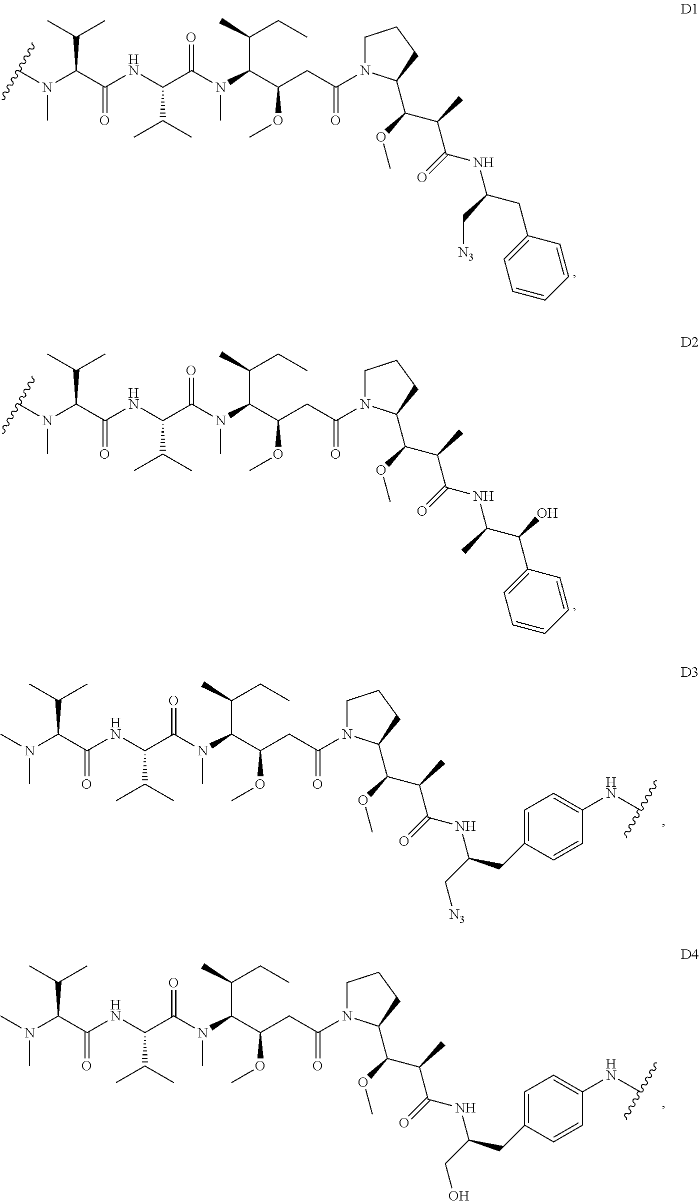

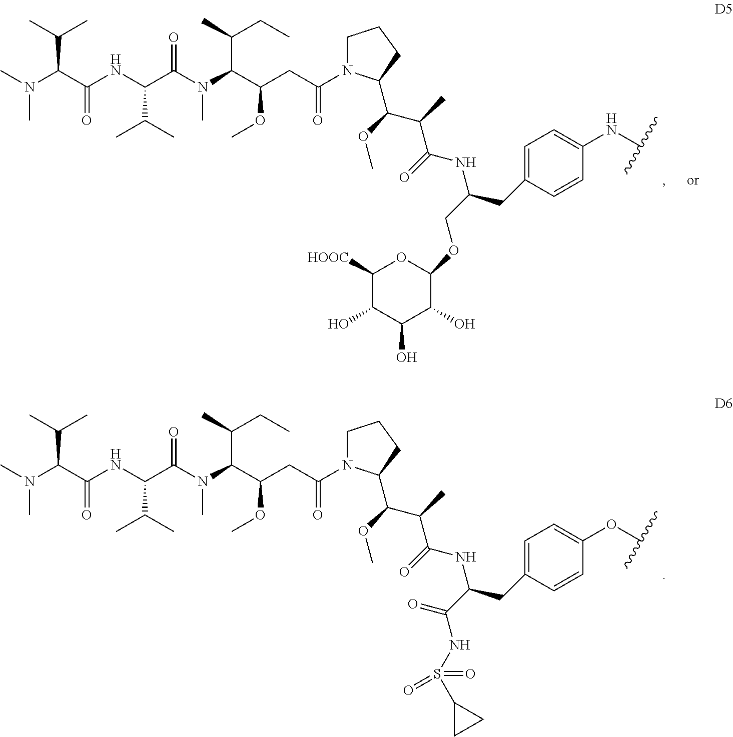 [SMILES: *Nc1ccc(C[C@@H](CO[C@@H]2O[C@H](C(=O)O)[C@@H](O)[C@H](O)[C@H]2O)NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)cc1 ; *Oc1ccc(C[C@H](NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)C(=O)NS(=O)(=O)C2CC2)cc1]

86. The method of claim 85, wherein D is

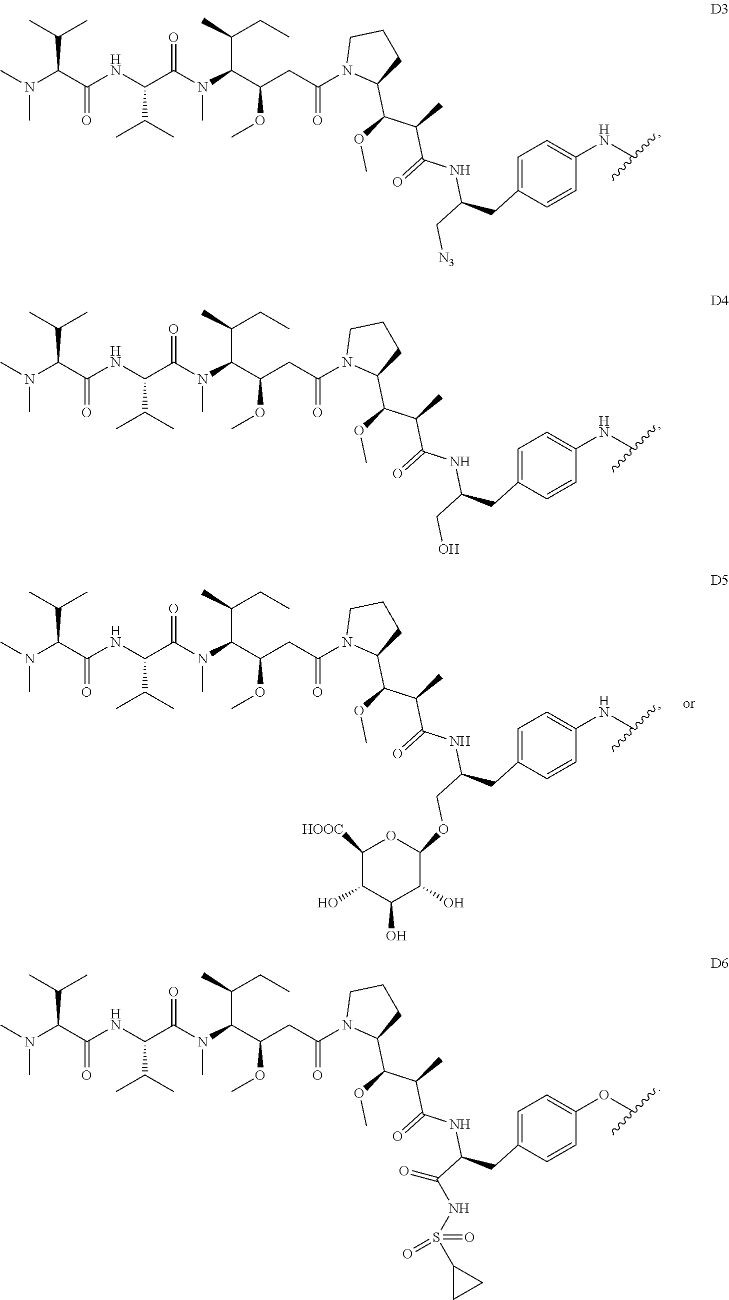

87. The method of claim 86, wherein D is

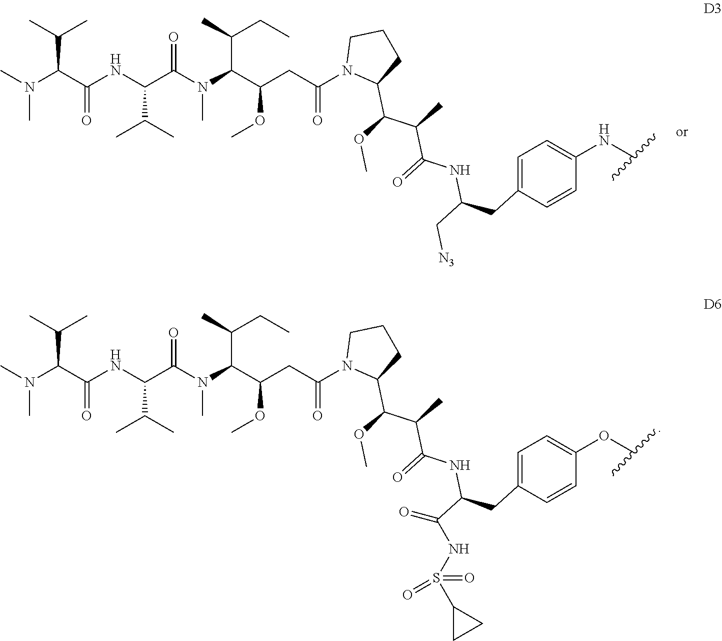 [SMILES: *Nc1ccc(C[C@@H](CN=[N+]=[N-])NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)cc1 ; *Oc1ccc(C[C@H](NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)C(=O)NS(=O)(=O)C2CC2)cc1]

88. The method of any one of claims 52-87, wherein B-L₂-D is: 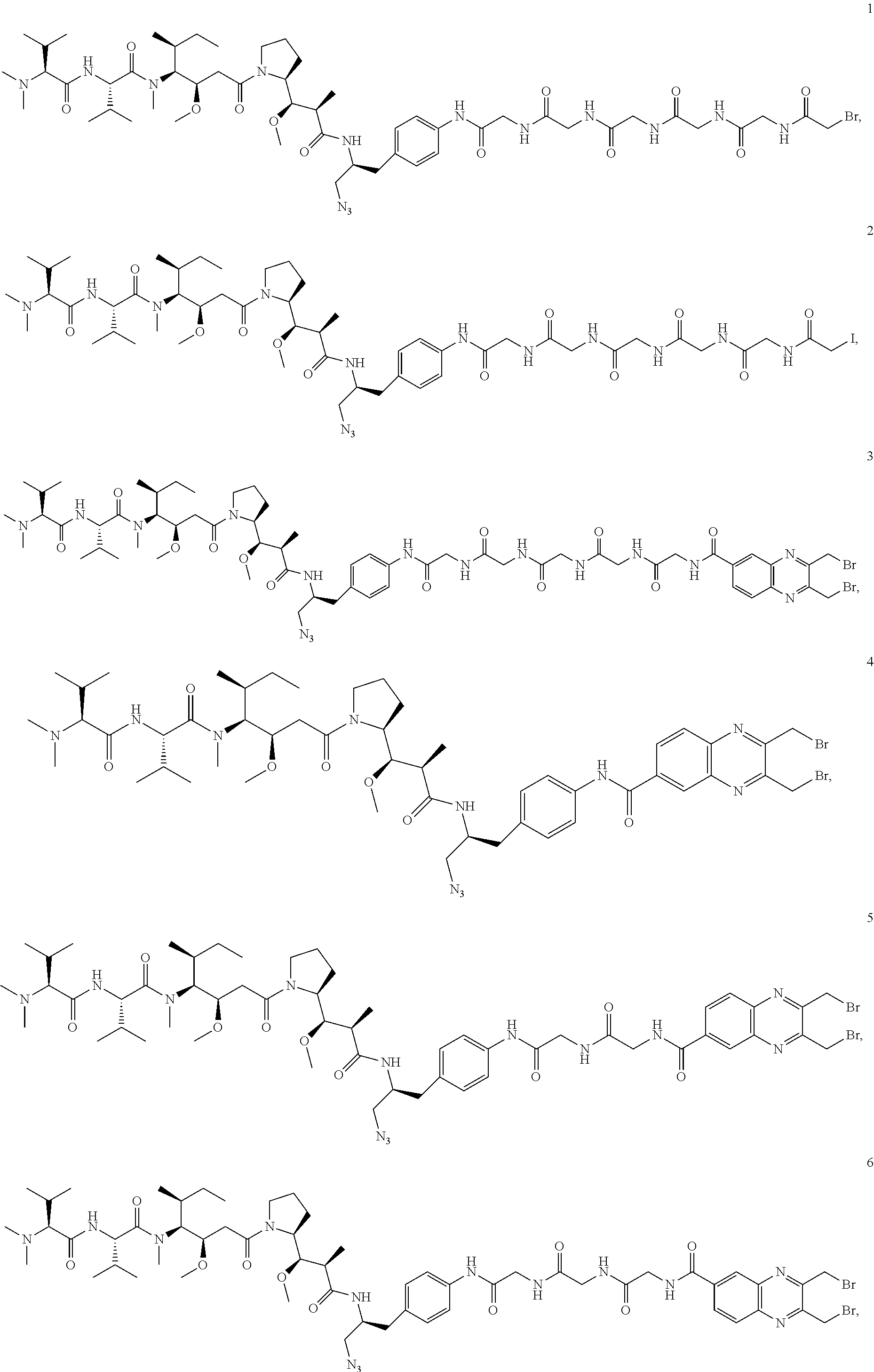 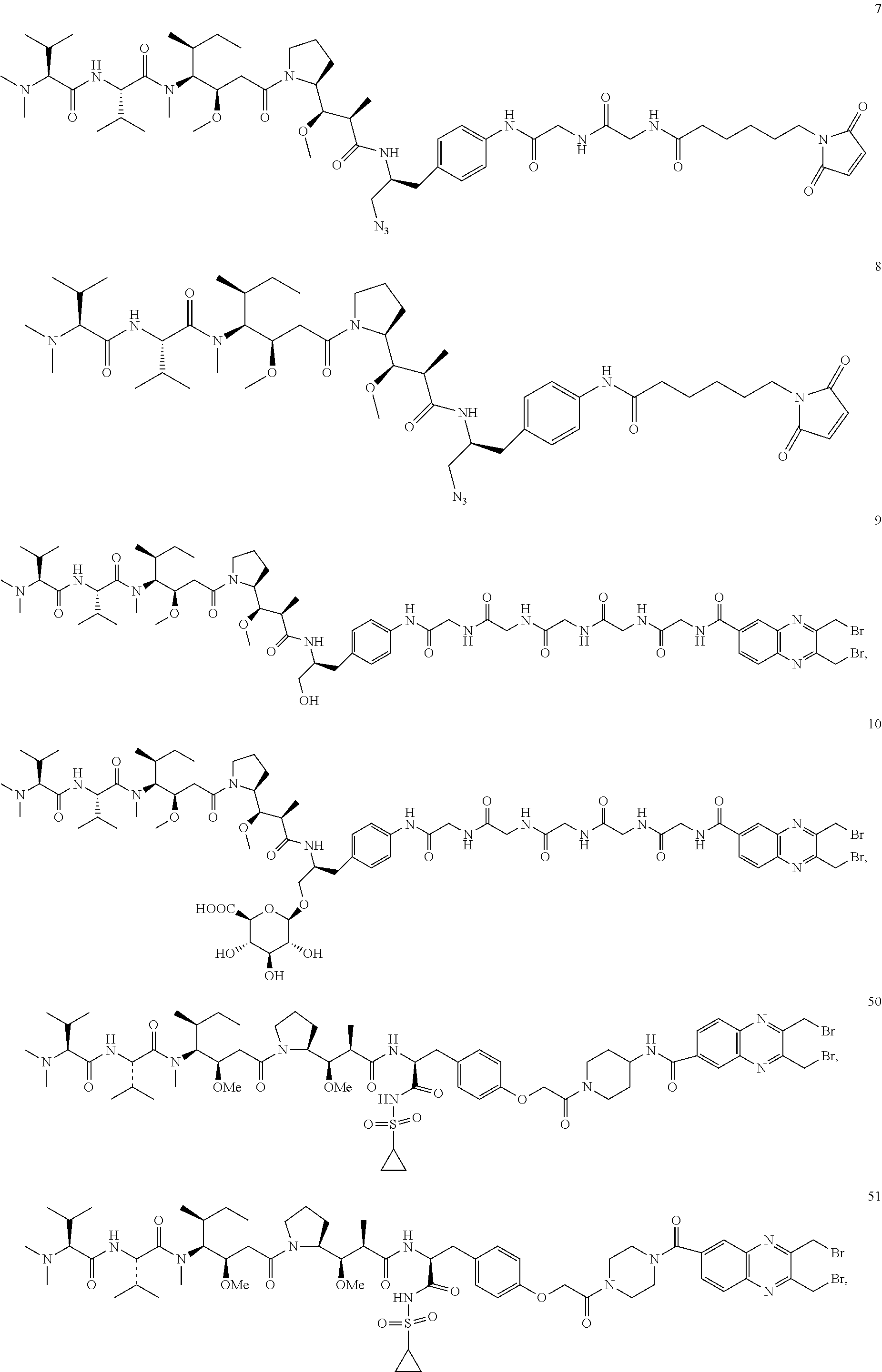 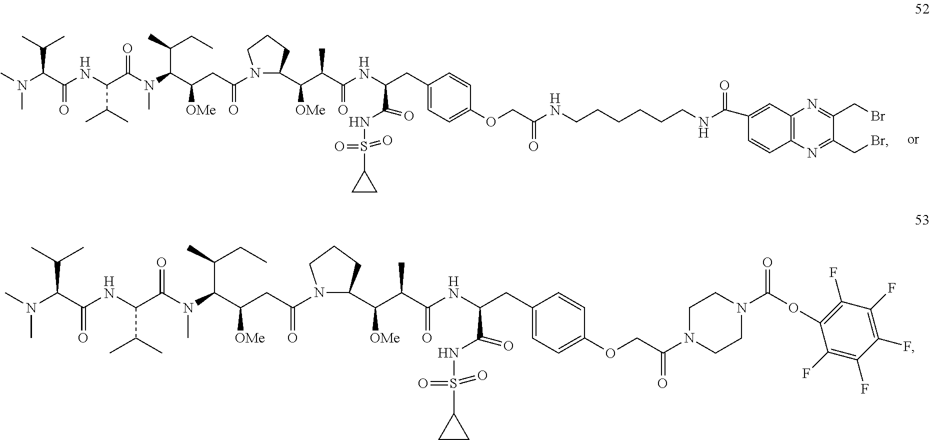 [SMILES: CC[C@H](C)[C@@H]([C@@H](CC(=O)N1CCC[C@H]1[C@H](OC)[C@@H](C)C(=O)N[C@@H](Cc1ccc(OCC(=O)NCCCCCCNC(=O)c2ccc3nc(CBr)c(CBr)nc3c2)cc1)C(=O)NS(=O)(=O)C1CC1)OC)N(C)C(=O)[C@@H](NC(=O)[C@H](C(C)C)N(C)C)C(C)C ; "or" ; CC[C@H](C)[C@@H]([C@@H](CC(=O)N1CCC[C@H]1[C@H](OC)[C@@H](C)C(=O)N[C@@H](Cc1ccc(OCC(=O)N2CCN(C(=O)Oc3c(F)c(F)c(F)c(F)c3F)CC2)cc1)C(=O)NS(=O)(=O)C1CC1)OC)N(C)C(=O)[C@@H](NC(=O)[C@H](C(C)C)N(C)C)C(C)C]
or a pharmaceutically acceptable salt thereof.

89. A compound of formula (II): 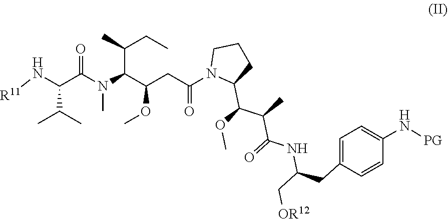
or a pharmaceutically acceptable salt thereof, wherein:
PG is an amine protecting group;
R¹¹ is H or one or more Amino Acid Units;
R¹² is H or a substituted alkyl, substituted heteroalkyl, substituted heterocycloalkyl, —CO(CH₂CH₂O)ₛCH₂CH₂U, or —CONH(CH₂CH₂O)ₛCH₂CH₂U; wherein
s is an integer from 1 to 24; and U is —NH₂, —OH, —COOH, or —OCH₃.

90. The compound of claim 89, wherein R¹² is H or substituted heterocycloalkyl.

91. The compound of claim 90, wherein R¹² is H or

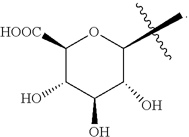

92. The compound of claim 91, wherein R¹² is H.

93. The compound of any one of claims 89-92, wherein R¹¹ is H or a hydrophobic amino acid.

94. The compound of any one of claims 89-93, wherein R¹¹ is H, valine, isoleucine, leucine, methionine, phenylalanine, alanine, L-norleucine, proline, tryptophan, 2-aminoisobutyric acid, or 3-cyclohexyl-L-alanine.

95. The compound of any one of claims 89-94, wherein R¹¹ is H.

96. The compound of any one of claims 89-95, wherein PG is Boc, Fmoc, or CBZ.

97. The compound of any one of claims 89-96, wherein PG is Boc.

98. The compound of any one of claims 89-97, wherein the compound is: 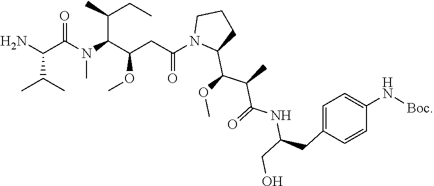 [SMILES: CC[C@H](C)[C@@H]([C@@H](CC(=O)N1CCC[C@H]1[C@H](OC)[C@@H](C)C(=O)N[C@H](CO)Cc1ccc(NC(=O)OC(C)(C)C)cc1)OC)N(C)C(=O)[C@@H](N)C(C)C]
